(19) United States
(12) Patent Application Publication
(10) Pub. No.: US 2023/0159629 A1
(43) Pub. Date: May 25, 2023

(54) COMPOSITIONS AND METHOD OF MODULATING GROWTH FACTOR FUNCTION

(71) Applicant: CASE WESTERN RESERVE UNIVERSITY, Cleveland, OH (US)

(72) Inventors: M. Edward Medof, Pepper Pike, OH (US); Devin Cao, Cleveland, OH (US); Daniel Counihan, Cleveland, OH (US); Shiva Sridar, Cleveland, OH (US)

(21) Appl. No.: 18/096,349

(22) Filed: Jan. 12, 2023

Related U.S. Application Data
(63) Continuation-in-part of application No. 17/487,409, filed on Sep. 28, 2021.
Application No. 17/487,409 is a continuation of application No. 13/350,402, filed on Jan. 13, 2012, now Pat. No. 11,130,801.
(60) Provisional application No. 61/432,498, filed on Jan. 13, 2011.
(60) Provisional application No. 63/298,869, filed on Jan. 12, 2022.

(51) Int. Cl. C07K 16/18; C07K 16/24; C07K 16/28; G01N 33/564; A61K 45/06; A61K 38/58; A61K 38/08; A61K 38/06; A61K 38/57

(52) U.S. Cl. CPC C07K 16/18; C07K 16/248; C07K 16/28; G01N 33/564; A61K 45/06; A61K 38/58; A61K 38/08; A61K 38/06; A61K 38/57; C07K 2317/76; A61K 2039/507

(57) ABSTRACT

A method of modulating growth factor responses of cells expressing C3a receptor (C3aR) and C5a receptor (C5aR) and at least one growth factor receptor includes administering to the cells a thrombin inhibitor.

### RELATED APPLICATION

This application is a Continuation-in-Part of U.S. patent application Ser. No. 17/487,409, filed Sep. 28, 2021, which is a Continuation of U.S. patent application Ser. No. 13/350,402, filed Jan. 13, 2012 (Now U.S. Pat. No. 11,130,801), which claims priority from U.S. Provisional Application No. 61/432,498, filed Jan. 13, 2011. This application also clams priority to U.S. Patent Application No. 63/298,869, filed Jan. 12, 2022, the subject matter of which are incorporated herein by reference in their entirety.

### GOVERNMENT FUNDING

This invention was made with government support under AR067182 AND HL109561 awarded by the National Institutes of Health. The government has certain rights in the invention.

### TECHNICAL FIELD

This application relates to a compositions and methods of modulating growth factor function and to compositions and methods of treating growth factor mediated disorders and conditions.

### BACKGROUND

The complement system is made up of several distinct plasma proteins that react with one another to opsonize pathogens and induce a series of inflammatory responses that help combat infection. The complement cascade can be activated on the surface of a pathogen through one or more of the three distinct pathways, the classical, the MB-Lectin, and the alternative. Each of these three pathways follows a sequence of reactions to generate proteases known as C3 convertases. These active proteases, bound covalently to the pathogen surface, are responsible for the cleavage of complement component C3 for the generation of large amounts of C3b, the main effector molecule of the complement system, and C3a, a peptide mediator of inflammation. The C3b molecules bind covalently to the pathogen, acting as opsonins, and target it for destruction by phagocytes prepared with receptors for C3b. The C3b also binds with C3 convertase to form C5 convertase, responsible for producing a second peptide mediator of inflammation, C5a, as well as C5b, initiator of the later events of complement activation.

Virtually all aspects of development, growth, cellular homeostasis, and tissue regeneration/repair are regulated by growth factors. As examples, angiogenesis depends on VEGF, epithelial cell growth on EGF, smooth muscle cell growth on PDGF, monocyte/macrophage growth on GM-CSF, and nerve growth on NGF. Each of these growth factors mediates its effects via ligation of specific receptor tyrosine kinases (RTKs). Under homeostatic conditions, tonic growth factor production and RTK signaling confers survival signals. In response to exogenous stimuli, e.g., hypoxia in the case of VEGF, amplified growth factor production and potentiated RTK signaling triggers the cell cycle and thereby induces proliferation. According to current concepts, RTK auto-phosphorylation resulting from engagement of its respective growth factor initiates downstream signaling cascades that confer its viability and mitotic effects directly.

Studies involving VEGF and its connection with components C3a and C5a have been shown to have clinical significance as well. Age-related macular degeneration (AMD) is the leading cause of permanent vision loss among the elderly in many industrialized countries. In a study conducted with retinal pigmented epithelium (RPE) cells, bioactive fragments of C3a and C5a were shown to be present in drusen of patients with AMD, and that C3a and C5a induce VEGF expression. Furthermore, these components were shown to be generated early in the course of laser-induced choroidal neovascularization (CNV), an accelerated model of neovascular AMD driven by VEGF and recruitment of leukocytes into the choroid.

### SUMMARY

This application relates to a method of modulating growth factor responses of cells expressing C3a receptor (C3aR) and C5a receptor (C5aR) and at least one growth factor receptor. The method includes administering to the cells at least one agent that modulates C3aR and/or C5aR signaling of the cells.

In some embodiments, the agent can inhibit C3aR and/or C5aR signaling of the cells and be administered to the cells at an amount effective to inhibit at least one of growth, viability, or mitosis of the cells. The agent that inhibits C3aR and/or C5aR signaling of the cells can be selected from the group consisting of a complement antagonist that substantially reduces the interaction of at least one of C3a or C5a with the C3a receptor (C3aR) and C5a receptor (C5aR), a STAT3/IL-6 signaling pathway antagonist (e.g., a STAT3 inhibitor and an IL-6 inhibitor), a thrombin inhibitor, and a combination thereof.

In some embodiments, the cells can include at least one of smooth muscle cells, endothelial cells, leukocytes, cancer cells, neural cells, or fibroblasts. The agent can inhibit at least one of growth, viability, or mitosis of the smooth muscle cells, endothelial cells, leukocytes, cancer cells, neural cells, or fibroblasts in response to growth factor stimulation.

In other embodiments, the agent can include a complement antagonist and the cells can be in vasculature of the subject, proximate or about the periphery of a vascular injury. The agent can be administered to the subject to inhibit at least one growth, viability, or mitosis of the cells following growth factor stimulation of the cells. The vascular injury can include at least one of atherogenesis, thrombosis, restinosis, or neointimal formation in the subject.

In still other embodiments, the cells can include tumor or cancer cells and the agent can being selected from the group consisting of a STAT3 inhibitor, an IL-6 inhibitor, a thrombin inhibitor and a combination thereof. The agent can be administered to the cells at an amount effective to inhibit at least one of growth, viability, or mitosis of the cells.

In yet other embodiments, the cells can be endothelial cells and the agent can be selected from the group consisting of a STAT3 inhibitor, an IL-6 inhibitor, a thrombin inhibitor and a combination thereof. The agent can be administered to the endothelial cells at an amount effective to inhibit at least one of growth, viability, or mitosis of the cells.

Another aspect of the application relates to a method of inhibiting neointimal formation in the vasculature of a subject as a result of a vascular injury. The method includes administering to at least one of endothelial cells or smooth muscle cells at the site of or proximate the site of the injured vasculature an agent that inhibits C3aR and/or C5aR signaling of the cells. The agent can be selected from the group consisting of a complement antagonist that substantially reduces the interaction of at least one of C3a or C5a with the C3a receptor (C3aR) and C5a receptor (C5aR).

In some embodiments, the agent can be administered at an amount effect to inhibit at least one of PDGF production by the cells, cell growth, cell viability, or cell mitosis. The agent can also be administered at an amount effective to inhibit at least one of atherogenesis, thrombosis, or restinosis of the subject.

In other embodiments, the agent can be provided on an endovascular device and the endovascular device being administered to the site of vascular injury. The endovascular device can include stents, drug delivery catheters, grafts, and drug delivery balloons utilized in the vasculature of a subject.

### BRIEF DESCRIPTION OF THE DRAWINGS

The foregoing and other features of the invention will become more apparent upon a consideration of the following description taken in connection with the accompanying drawings wherein:

FIG. 1 is a schematic diagram illustrating growth factor and amplification through complement receptor signaling.

FIG. 2 is a schematic diagram illustrating that when C3aR/C5aR signal transduction is antagonized either pharmacologically, immunologically, or genetically, cell growth and progression from G0 to G2 is blocked.

FIG. 3 illustrates a chart showing the effect of C3aR/C5aR antagonism on 7 RTKs and f31-adreneric receptor in respective cell types.

FIG. 4 illustrates a chart showing autocrine C3aR/C5aR signaling is essential for EC viability. bEnd.3 and MS-1 cells were incubated for 24 hours in EC growth medium, and locally produced C3a and C5a in culture supernatants quantitated by ELISA.

FIG. 5 illustrates a chart showing Bcl-2, Bclx-L, Bax, and Bim mRNA expression levels in bEnd.3 cells incubated for 8 hr with C3aR-A/C5aR-A (10 ng/ml each) or anti-C3a/C5a (1 ug/ml each).

FIG. 6 illustrates a chart showing Annexin V positivity of bEnd.3 cells and MS-1 cells administered C3aR-A/C5aR-A (10 ng/ml each).

FIG. 7 illustrates a chart showing complement mRNA transcription (Left, data shown for C3 mRNA) in HUVEC cells following C3a or C5a administration or hypoxia. Hypoxia was induced by FCCP and IAA in HUVEC for 1 hr and C3 mRNA levels were quantified by qPCR (Right).

FIG. 8 illustrates plots showing growth of bEnd.3 cells following administration of C3aR-A/C5aR-A signaling and/or VEGF.

FIG. 9 illustrates plots showing growth of MS-1 cells following administration of C3aR-A/C5aR-A signaling and/or VEGF.

FIG. 10 illustrates plots showing growth of HUVECs incubated with C3aR-A/C5aR-A (10 ng/ml each) or anti-C3/anti-C5 mAbs (1 μg/ml each) at 24, 28 and 72 hr assayed.

FIG. 11 illustrates plots showing expression of C3 and C5 mRNA of bEnd.3 cells incubated for 1 hr with VEGF-A (30 ng/ml).

FIG. 12 illustrates a chart showing C3a/C5a production of bEnd.3 cell or MS-1 cells treated with VEGF and C3aR-A/C5aR-A.

FIG. 13 illustrates a chart showing Annexin V positivity in primary ECs added of C3aR-A/C5aR-A (10 ng/ml each).

FIG. 14 illustrates a chart showing C3, fB, fD, C5, C3aR, and C5aR levels of primary ECs were incubated for 30 min with VEGF-A (30 ng/ml).

FIG. 15 illustrates images showing primary ECs isolated from aortic rings of Daf1^{-/-}, WT and C3aR^{-/-}C5aR^{-/-} mice grown in EC growth medium for 2 wk and photographed following the first passage.

FIG. 16 illustrates a plot showing cell numbers of 5×10⁵ ECs of each genotype cultured in EC growth medium after 24, 48, and 72 hr.

FIG. 17 illustrates a chart showing mRNA expression of C3, fB, fD, and C5 by cells from whole aorta of each identified genotype.

FIG. 18 illustrates plots showing growth of WT and C3aR^{-/-}C5aR^{-/-} incubated with VEGF-A (30 ng/ml) at 24, 48, and 72 hr assayed.

FIG. 19 illustrates images showing autocrine C3aR/C5aR signaling in ECs is essential for HUVEC tube formation and corneal neovascularization. HUVEC were plated with EBM-2 Basal Medium without supplemental growth factors, with VEGF-A, or with VEGF-A plus C3aR-A/C5aR-A.

FIG. 20 illustrates images showing male mice were injected subcutaneously with 1×10⁶ RM1 prostate cancer cells. Tumors were collected 10 days after injection and were weighed (Top, n=8). Representative images of immunostaining for CD31 in tumor sections of WT, Daf1^{-/-}, and C3R^{-/-}C5aR^{-/-} mice, showing CD31 expression in new vessels (Middle). CD31-positive areas were quantified in 5-10 independent fields per tumor implant (Bottom).

FIG. 21 illustrates an immunoblot of VEGFR2 phosphorylation. C5a or VEGF-A was added to serum starved primary cultures of WT murine ECs in the absence or presence of C3aR-A/C5aR-A and VEGFR2 phosphorylation assessed by immunoblotting with anti p-T1094/T1095 mAb.

FIG. 22 illustrates a chart showing quantitation of bands in FIG. 21.

FIG. 23 illustrates a plot showing the expression of VEGF from the second passage of primary ECs from WT and C3R^{-/-}C5aR−/−.

FIG. 24 illustrates the expression of VEGFR2 of primary ECs starved in DMEM/F12 with 0.5% FBS for 24 hrs. The supernatants and cell lysates were collected and analyzed for VEGF content by ELISA. The results were normalized by protein concentration.

FIG. 25 illustrates plots showing growth of serum starve primary cultures of WT murine ECs in the absence or presence of SU5416 or C5a. The cell proliferation was quantified by Trypan blue exclusion.

FIG. 26 illustrates a chart showing the expression of Integrinβ3 from the passage 3-4 of primary EC of WT and C3R^{-/-}C5aR^{-/-} measured by FACS.

FIG. 27 illustrates plots showing the growth of ECs in response to VEGF induces C3aR/C5aR signaling via an IL-6 and Stat3 dependent mechanism. VEGF-A was added to serum starved MS-1 cells in the absence or presence of anti-IL-6 neutralizing mAb (2 μg/ml) and growth was quantified at 24, 28 and 72 hr.

FIG. 28 illustrates plots showing the growth of serum starved MS-1 cells in the absence or presence of C3aR-A/C5aR-A (10 ng/ml each) and IL-6 (10 ng/ml) at 24, 48 and 72 hr.

FIG. 29 illustrates a chart showing phosphor-Stat3 of serum starved MS-1 cells incubated for 10 min at 37° C. with VEGF-A or IL-6 in the absence or presence of anti-IL-6 mAb or C3aR-A/C5aR-A.

FIGS. 30(A-B) illustrate plots showing (A) NIH-3T3 cells were incubated as indicated cell counted over 72 hrs. (B) CTLL IL-2 dependent cells were incubated as indicated and counted over 72 hrs.

FIGS. 31(A-B) illustrate charts showing: (A) C5a (17 ng/mL) added to NIH-3T3 cells. Bars represent 72 hr counts. (B) PDGF-AA was added to NIH-3T3 cells and a 72 hr culture supernatants were assayed for C5a by ELISA.

FIG. 32 illustrates plots showing SMCs from different knockouts were incubated with PDGF-AA and cell numbers determined each day.

FIGS. 33(A-D) illustrate charts showing qRT-PCR analysis of C3, factor B and factor D transcripts from HUVEC under hypoxia. A) HUVEC treated with TNF-α, IL-1, and IFN-γ. Blue bars represent samples after treatment. B-C) HUVEC stimulated with C3a (10 ng/ml for 2 hr) or C5a (10 ng/ml for 30 min.) Blue bars represent control without C3a/C5a stimulation while red bars represent samples after stimulation. D) HUVEC incubated for 1 hr with FCCP+IAA. Blue bars represent control without hypoxia treatment while yellow bars represent two samples after hypoxia treatment.

FIG. 34 illustrates a chart showing HUVECs stimulated with simvastin, and assayed for DAF and KLF4 mRNA.

FIGS. 35(A-D) illustrates images and charts showing: A) Verhoeffelastin stain 14 day after femoral artery wire injury (original magnification 10×). B) Intima area:media area ratio 14 d after injury. C) Medical Leukocyte (% CD45-positive cells) accumulation 14 d after injury. D) Cellular proliferation (% BrdU-positive cells) in the media 14 day after injury.

FIGS. 36(A-E) illustrate vascular endothelial cell growth factor A (VEGF-A) induces intracellular synthesis of the components of prothrombinase. All experiments were typically done three times but in some cases at least twice. A: bEnd.3 cells were incubated for 1 hour at 37° C. without or with VEGF-A (30 ng/mL) and mRNA expression of prothrombinase components [coagulation factors II (FII), V (FV), X (FX), and VII (FVII)] assayed by real-time quantitative PCR (qPCR). mRNA was normalized to b-actin. B: bEnd.3 cells were incubated for 72 hours at 37° C. with media alone (0.05% fetal bovine serum) or with VEGF-A (30 ng/mL), after which their culture supernatants were assayed for their abilities to accelerate the clotting times of human plasmas deficient in FII, FV, FVII, or FX (media control without cells was <0.01 U/mL). C: Human aortic endothelial cell (HAECs) were incubated for 72 hours at 37° C. without or with VEGF-A (30 ng/mL), and coagulation assays were performed as in B (media control was <0.01 U/mL). D: RNA from thoroughly perfused aortae of wild-type (WT) mice was assayed for tissue factor, FII, FV, and FX mRNA levels by qPCR. E: Matrigel plugs were implanted into the flanks of WT mice for 10 days. Plugs were harvested, embedded in OCT compound, and sectioned. Sections were stained with anti-FII, anti-FV, anti-FVII, or anti-FX (each red) and counterstained with anti-CD31 (green). Nuclei were visualized with DAPI (blue). Background signaling was minimal and suppressed. The data are representative of six separate images. *P<0.05.

FIGS. 37(A-G) illustrate vascular endothelial cell growth factor A (VEGF-A) induces intracellular generation of C5a, C5a receptor-1 (C5ar1), and thrombin. A: bEnd.3 cells were incubated for 72 hours at 37° C. without or with VEGF-A (30 ng/mL) alone, VEGF-A (30 ng/mL) [symbol] bivalirudin (Angiomax; 25 mg/mL), or VEGF-A [symbol] antithrombin (1 mmol/mL), and cell numbers were counted at 24, 48, and 72 hours. The 0.05% fetal bovine serum (FBS) was used to exclude effects of other growth factors or mediators in 10% FBS. Antithrombin can inhibit VEGF-A binding to VEGF receptor 2 but only after its cleavage, resulting from its inactivation of thrombin. B: bEnd.3 cells were incubated for 72 hours at 37° C. without or with VEGF-A (30 ng/mL), or VEGF-A [symbol] antithrombin (1 mmol/mL), after which C5a in culture supernatants was assayed by enzyme-linked immunosorbent assay. C and D: bEnd.3 cells (C) and human aortic endothelial cells (HAECs; D) were incubated with VEGF-A (30 ng/mL) for 24 hours, after which intracellular and extracellular C5a and C5ar1 were measured by flow cytometry. E: bEnd.3 cells were incubated for 48 hours at 37° C. with VEGF-A (30 ng/mL), after which cells were assayed for anti-total factor II (FII) reactivity extracellularly and intracellularly via flow cytometry. F: bEnd.3 cells were incubated for 48 hours at 37° C. with VEGF-A (30 ng/mL)±C3ar1-A/C5ar1-A (10 ng/mL each) (RA □ combined C3ar1 and C5ar1 antagonists), after which cells were assayed for anti-total FII reactivity extracellularly and intracellularly via flow cytometry. G: HAECs were pre-incubated for 2 days in serum-free media, after which the cells were assayed for specific anti-thrombin reactivity intracellularly via flow cytometry. *P<0.05, ***P<0.001. MFI, mean fluorescence intensity; N.D., not detectable.

FIGS. 38(A-F) illustrate combinatorial C5a receptor-1 (C5ar1) and protease-activated receptor signaling is required for endothelial cell (EC) growth. All experiments were performed two or more times. A: Human aortic ECs (HAECs) were incubated for 72 hours at 37° C. without or with vascular endothelial cell growth factor A (VEGF-A; 30 ng/mL) or C5a (30 ng/mL), and cell numbers were counted at 0, 24, 48, and 72 hours. B: HAECs were incubated for 72 hours at 37° C. without or with VEGF-A (30 ng/mL) alone, VEGF-A [symbol] antithrombin (1 mmol/mL), or VEGF-A [symbol] antithrombin [symbol] C5a (10 ng/mL), and cell numbers were counted at 0, 24, 48, and 72 hours. C: HAECs were incubated at 37° C. without or with VEGF-A (30 ng/mL) alone, VEGF-A [symbol] C3ar1-A/C5ar1-A (10 ng/mL each), thrombin (1 nmol/mL), or thrombin [symbol] C3ar1-A/C5ar1-A, and cell numbers were counted over 3 days. D: HAECs were incubated at 37° C. with VEGF-A (30 ng/mL), VEGF-A (30 ng/mL) [symbol] vorapaxar (10 ng/mL), or VEGF-A (30 ng/mL) [symbol] vorapaxar (10 ng/mL) [symbol] C5a (10 ng/mL), and cell numbers were counted as in D. E: HAECs were incubated for 72 hours at 37° C. without or with TP508 (10 mg/mL) or TP508 (10 mg/mL) plus C5a (10 ng/mL), and cell numbers were counted at 0, 24, 48, and 72 hours. F: The experiment in E was repeated, substituting TFLLRN (5 mg/mL) for TP508.

FIGS. 39(A-E) illustrate autocrine protease-activated receptor (PAR) 1/4 signaling is required for vascular endothelial cell growth factor A (VEGF-A) mitotic function, and endothelial cell (EC) prothrombinase and complement production depend on each other. A: Human aortic ECs (HAECs) were treated with siRNA targeting either PAR1 or PAR4 (or nonspecific control, Silencer) for 7 days. Following confirmation of knockdown, cells were pre-incubated for 16 hours in serum-free medium and then treated with VEGF-A (30 ng/mL) or C5a (30 ng/mL) for 3 days (in serum-free media), and growth was measured. As our studies have shown that VEGF receptor 2 (VEGFR2) signaling depends on PAR1 and PAR4 signaling (FIG. 37), the loss of the VEGF-A response likely involved reduced VEGFR2 expression. B: HAECs were incubated for 1 hour at 37° C. without or with C5a (10 ng/mL) or C5a [symbol] plus antithrombin (1 mmol/mL), and cells were assayed for factor II (FII), factor V (FV), factor VII (FVII), or factor X (FX) mRNAs by real-time quantitative PCR (qPCR). mRNA was normalized to glyceraldehyde-3-phosphate dehydrogenase (GAPDH). The anti-C5a monoclonal antibody (mAb) recognizes a neoepitope not present in the parental C5 protein. C: HAECs were incubated for 1 hour at 37° C. without and with VEGF-A (30 ng/mL), or VEGF-A [symbol] anti-C5a mAb (1 mg/mL), and cells were assayed for FII, FV, FVII, and FX mRNAs by qPCR. mRNA was normalized to GAPDH. D: HAECs were incubated for 48 hours with VEGF-A (30 ng/mL)±C3ar1-A/C5ar1-A (10 ng/mL each), after which cells were assayed for anti-total FII reactivity extracellularly and intracellularly by flow cytometry. E: HAECs were incubated for 1 hour at 37° C. without or with thrombin (1 nmol/mL), and cells were assayed for C3, C5, C3ar1, and C5ar1 mRNAs by qPCR. mRNA was normalized to GAPDH. *P<0.05.

FIGS. 40(A-G) illustrate C5a receptor-1 C5ar1) and protease-activated receptors (PARs) closely interact, and phospholipase C (PLC), AKTeglycogen synthase kinase (GSK)-3b, and STAT3 activation depends on their joint signaling. All experiments were performed two or more times. A: Human 293 cells cotransfected with C5ar1egreen fluorescent protein (GFP) [symbol] PAR4-luciferase (Luc), PAR4-GFP [symbol] C5ar1-Luc, or alternatively with C5ar1-GFP [symbol] PAR1-Luc, or PAR1-GFP [symbol] C5ar1-Luc. Cells also were transfected with PAR4-GFP [symbol] PAR4-Luc as a positive control. Bioluminescence resonance energy transfer (BRET) in each combination was measured after addition of Luc substrate. B: Anti-PAR4 immunoprecipitations (IPs), anti-C5ar1 IPs, and anti-CD59 IPs were prepared from bEnd.3 cell extracts, and immunoblots of the immunoprecipitated proteins were probed for associated C5ar1, PAR4, or CD59. C: bEnd.3 cells in complete media were incubated with vascular endothelial cell growth factor A (VEGF-A; 30 ng/mL) for 1 hour, after which the fixed and permeabilized cells were stained with antieC5ar1-GFP and antiePAR4-RFP and examined by confocal microscopy. Background signaling was minimal and suppressed. The data are representative of six separate images. Arrows represent sites of colocalization. D: Left panel: Human umbilical vein endothelial cells (HUVECs) were stimulated with C5a or C5a plus bivalirudin (Angiomax) or vorapaxar for 5 minutes, after which cells were lysed and assayed for PLC activity. Right panel: HUVECs were stimulated with thrombin or thrombin plus C3ar1-A/C5ar1-A (RA) for 5 minutes, after which cells were lysed and assayed for protein kinase A (PKA) activity. E: Serum-starved bEnd.3 cells were incubated for 72 hours at 37° C. without or with VEGF-A (30 ng/mL), VEGF-A [symbol] the PI-3Kg inhibitor AS252424 (10 mmol/mL), or VEGF-A [symbol] C3ar1-A/C5ar1-A (RA; 10 ng/mL), after which cell extracts were assayed for GSK-3b activity. F: The protocol in E was used substituting C5a (10 ng/mL) for VEGF-A and including C5a [symbol] the PI-3Kg inhibitor AS252424 (10 mg/mL) and C5a [symbol] the VEGFR2 inhibitor SU5416 (10 mmol/mL). G: bEnd.3 cells were incubated with VEGF-A (30 ng/mL), thrombin (1 mmol/mL), or C5a (10 ng/mL) in the absence or presence of C5ar1-A (RA; 10 ng/mL) or antithrombin (1 mmol/mL), after which the cells were assayed for phosphorylated STAT3 (p-STAT3) by intracellular flow cytometry. *P<0.05, ***P<0.001. AU, arbitrary unit; CPM, counts per minute; IB, immunoblot.

FIGS. 41(A-E) illustrate role of C3ar1, protease-activated receptors (PAR) 1/PAR4, and vascular endothelial cell growth factor receptor 2 (VEGFR2) signaling in activation of PI-3Kγ and PI-3Ka. A and B: bEnd.3 cells were incubated for increasing times at 37° C. with thrombin (1 mmol/mL) without or with C3ar1-A/C5ar1-A (10 ng/mL each), after which antiePI-3Kg (A) and antiePI-3Ka (B) immunoprecipitations (IPs) of the cell extracts were assayed for PI-3K enzymatic activity. C: TFLLRN (PAR1 agonist) or ay-NH2 (PAR4 agonist) was titrated into bEnd.3 cells for 9 minutes at 37° C., after which PI-3K isoform activity was assayed, as in A and B above. D: Thrombin (1 mmol/mL; left panel) or C5a (10 ng/mL; right panel) was titrated into serum-starved bEnd.3 cells for 9 minutes, after which PI-3K isoform activity was assayed as above. E: Vascular endothelial cell growth factor A (VEGF-A), TP508, ay-NH2, and C5a were incubated with bEnd.3 cells for 9 minutes, cells were lysed, and anti-VEGFR2 IPs were prepared. The VEGFR2 IPs were eluted using alkaline solution, and the eluents were immunoprecipitated with PI-3Kα or PI-3Kγ Abs, after which PI-3K activity in the IPs was assayed as above.

FIGS. 42(A-H) illustrate combinatorial C5a receptor-1 (C5ar1) and thrombin agonism drives angiogenesis. The experiments were performed two or more times. A: Human aortic endothelial cell (HAECs) grown at 70% and 100% confluence were assayed for factor II (FII), factor V (FV), factor VII (FVII), factor X (FX), C3, and C5 mRNAs by real-time quantitative PCR. mRNA was normalized to glyceraldehyde-3-phosphate dehydrogenase. B: Human umbilical vein endothelial cells (HUVECs) grown at 70% or 100% confluence were stained with antibodies against FII (red), counterstained with DAPI (blue) and CD31 (data not shown), and imaged by confocal microscopy. Three randomly selected fields of view under each condition are presented. C: HUVECs grown at 70% or 100% confluence were stimulated with vascular endothelial cell growth factor A (VEGF-A; 30 ng/mL) or media control for 24 hours, after which C5a in the culture supernatant was assayed by enzyme-linked immunosorbent assay. D: Representative sections of aortic segments from wild-type (WT) mice were incubated for 7 days in Matrigel with VEGF-A (30 ng/mL), VEGF-A [symbol] C3ar1-A/C5ar1-A (RA; 10 ng/mL each), or VEGF-A [symbol] antithrombin (1 mmol/mL). E: Quantitation of sprout areas from WT aortic segments grown without or with VEGF-A, VEGF-A [symbol] C3ar1-A/C5ar1-A, or VEGF-A [symbol] antithrombin (each point represents growth from one aortic ring; three rings were isolated per mouse). These analyses were performed four times. F: Identical studies were done for aortic segments from WT mice treated with VEGF-A (30 ng/mL)±vorapaxar (10 ng/mL), aortic segments from C5ar1^{e/e} mice treated with VEGF-A (30 ng/mL), and aortic segments from Par4^{e/e}Par3^{e/e} and Par1^{e/e} mice treated with VEGF-A (30 ng/mL). These studies were performed three times. G: Diagram of C5ar1eprotease-activated receptor (PAR) 4 interaction at confluence versus when cells are growing. H: Diagram of PAR and C5ar1 Gbg activation of PI-3Kg and C5ar1 PI-3Ka via transactivation to VEGF receptor 2 (VEGFR2). PAR Gaq activation of phospholipase C (PLC) and C5ar1 Gai repression of protein kinase A (PKA) also are depicted. The proposed activation sequence is as follows: 1, extracellular stimulus (hypoxia/thrombin); 2, thrombin cleavage of endothelial cell C5 and PAR1/4; 3, PAR1/4/C5ar1 Gbγ activation of PI-3Kγ and phosphorylated AKT; 4, C5ar1 transactivation to VEGFR216, 5, VEGFR2 activation of PI-3Ka; 6, C5ar1/PAR4 induced [ PLC and Y PKA; 7, PI3K-AKT-mTOR, diacylglycerol/IP₃, and STAT3 signaling, and 8, amplified C5ar1/PAR4/VEGFR2 signaling. n=2 (E). *P<0.05, **P<0.01, and ***P<0.001.

FIGS. 43(A-K) illustrate combinatorial C5a receptor-1 (C5ar1) and protease-activated receptor (PAR) 1/PAR4 signaling is required for endothelial cell (EC) growth. A: bEnd.3 cells were incubated for 72 hours at 37° C. without or with vascular EC growth factor A (VEGF-A; 30 ng/mL) or C5a (30 ng/mL), after which cell numbers were counted at 0, 24, 48, and 72 hours. B: bEnd.3 cells were incubated for 72 hours at 37° C. without or with VEGF-A (30 ng/mL), VEGF-A+antithrombin (1 μmol/mL), or VEGF-A+antithrombin+C5a (10 ng/mL), after which cell numbers were counted at 0, 24, 48, and 72 hours. C: bEnd.3 cells were incubated without or with VEGF-A (30 ng/mL), VEGF-A+C3ar1-A/C5ar1-A (10 ng/mL each), thrombin (1 nmol/mL), or thrombin (1 μmol/mL)+C3ar1-A/C5ar1-A, after which cell numbers were counted over 3 days. D: bEnd.3 cells were incubated for 72 hours at 37° C. without or with VEGF-A (30 ng/mL) or VEGF-A (30 ng/mL)+vorapaxar (10 ng/mL), and cell numbers were counted at 0, 24, 48, and 72 hours. E: bEnd.3 cells were incubated as in A with a fourth condition: C5a (10 ng/mL) included VEGF-A and vorapaxar. F: bEnd.3 cells were incubated for 72 hours at 37° C. without or with VEGF-A (30 ng/mL) or C5a (10 ng/mL), and cell numbers were counted at 0, 24, 48, and 72 hours. G: bEnd.3 cells were incubated for 72 hours at 37° C. without or with TP508 (10 μg/mL) or TP508 (10 μg/mL) plus C5a (10 ng/mL), after which cell numbers were counted at 0, 24, 48, and 72 hours. H: The experiment in G was repeated, substituting TFLLRN (5 μg/mL) for TP508. I: bEnd.3 cells were incubated for 1 hour at 37° C. without or with C5a (10 ng/mL) or C5a+plus antithrombin (1 μmol/mL), after which cells were assayed for factor II (FII), factor V (FV), factor VII (FVII), and factor X (FX) mRNAs by real-time quantitative PCR (qPCR). J: bEnd.3 cells were incubated for 1 hour at 37° C. without and with VEGF-A (30 ng/mL), or VEGF-A+anti-C5a mAb (1 μg/mL), after which cells were assayed for FII, FV, FVII, and FX mRNAs by qPCR. K: bEnd.3 cells were incubated for 1 hour at 37° C. without or with thrombin (1 μmol/mL), and cells were assayed for C3, C5, C3ar1, and C5ar1 mRNAs by qPCR. *P<0.05.

FIG. 44 illustrates knockdown of protease-activated receptor (PAR) 1 and PAR4 expression. Human aortic endothelial cells were treated with siRNAs that target either PAR1 or PAR4 (or nonspecific control, Silencer) for 7 days, and PAR1 and PAR4 expression levels, respectively, were measured by flow cytometry as well as real-time quantitative PCR (data not shown). **P<0.001 and ***P<0.01. MFI, mean fluorescence intensity.

FIGS. 45(A-C) illustrate A) CD4⁺ cells were incubated for 72 h with anti-CD3/28 Dynabeads±C3ar1-A/C5ar1-A (10 ug/ml) or Angiomax (25 ug/ml) after which cell counts were measured. B) A similar experiment was performed assaying C5a (left) or IFN-γ (right) release into the supernatant. C) OT-II T cells were incubated for 72 h with DCs and ova₂₂₃₋₃₃₉ peptide±.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994. The definitions provided herein are to facilitate understanding of certain terms used frequently herein and are not meant to limit the scope of the application described herein.

As used herein, the term “polypeptide” refers to an oligopeptide, peptide, or protein sequence, or to a fragment, portion, or subunit of any of these, and to naturally occurring or synthetic molecules. The term “polypeptide” also includes amino acids joined to each other by peptide bonds or modified peptide bonds, i.e., peptide isosteres, and may contain any type of modified amino acids. The term “polypeptide” also includes peptides and polypeptide fragments, motifs and the like, glycosylated polypeptides, and all “mimetic” and “peptidomimetic” polypeptide forms.

As used herein, the term “polynucleotide” refers to oligonucleotides, nucleotides, or to a fragment of any of these, to DNA or RNA (e.g., mRNA, rRNA, tRNA) of genomic or synthetic origin which may be single-stranded or double-stranded and may represent a sense or antisense strand, to peptide nucleic acids, or to any DNA-like or RNA-like material, natural or synthetic in origin, including, e.g., iRNA, siRNAs, microRNAs, and ribonucleoproteins. The term also encompasses nucleic acids, i.e., oligonucleotides, containing known analogues of natural nucleotides, as well as nucleic acid-like structures with synthetic backbones.

As used herein, the term “antibody” refers to whole antibodies, e.g., of any isotype (IgG, IgA, IgM, IgE, etc.), and includes fragments thereof which are also specifically reactive with a target polypeptide. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain polypeptide. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term “antibody” also includes polyclonal, monoclonal, or other purified preparations of antibodies, recombinant antibodies, monovalent antibodies, and multivalent antibodies. Antibodies may be humanized, and may further include engineered complexes that comprise antibody-derived binding sites, such as diabodies and triabodies.

As used herein, the term “complementary” refers to the capacity for precise pairing between two nucleobases of a polynucleotide and its corresponding target molecule. For example, if a nucleobase at a particular position of a polynucleotide is capable of hydrogen bonding with a nucleobase at a particular position of a target polynucleotide (the target nucleic acid being a DNA or RNA molecule, for example), then the position of hydrogen bonding between the polynucleotide and the target polynucleotide is considered to be complementary. A polynucleotide and a target polynucleotide are complementary to each other when a sufficient number of complementary positions in each molecule are occupied by nucleobases, which can hydrogen bond with each other. Thus, “specifically hybridizable” and “complementary” are terms which can be used to indicate a sufficient degree of precise pairing or complementarity over a sufficient number of nucleobases such that stable and specific binding occurs between a polynucleotide and a target polynucleotide.

As used herein, the term “subject” refers to any warm-blooded organism including, but not limited to, human beings, rats, mice, dogs, goats, sheep, horses, monkeys, apes, rabbits, cattle, etc.

As used herein, the terms “complement polypeptide” or “complement component” refer to a polypeptide (or a polynucleotide encoding the polypeptide) of the complement system that functions in the host defense against infections and in the inflammatory process. Complement polypeptides constitute target substrates for the complement antagonists provided herein.

As used herein, the term “complement antagonist” refers to a polypeptide, polynucleotide, or small molecule capable of substantially reducing or inhibiting the activity of a complement component.

A complement component can include any one or combination of interacting blood polypeptides or glycoproteins. There are at least 30 soluble plasma polypeptides, in addition to cell surface receptors, which can bind complement reaction products and which can occur on inflammatory cells and cells of the immune system. In addition, there are regulatory membrane proteins that can protect host cells from accidental complement attack. Complement components can include polypeptides that function in the classical pathway, such as C2, polypeptides that function in the alternative pathway, such as Factor B, and polypeptides that function in the lectin pathway, such as MASP-1.

Complement components can also include: any of the “cleavage products” (also referred to as “fragments”) that are formed upon activation of the complement cascade; complement polypeptides that are inactive or altered forms of complement polypeptides, such as iC3 and C3a-desArg; and components indirectly associated with the complement cascade. Examples of such complement components can include, but are not limited to, C1q, C1r, C1s, C2, C3, C3a, C3b, C3c, C3dg, C3g, C3d, C3f, iC3, C3a-desArg, C4, C4a, C4b, iC4, C4a-desArg, C5, C5a, C5a-des-Arg, C6, C7, C8, C9, MASP-1, MASP-2, MBL, Factor B, Factor D, Factor H, Factor I, CR1, CR2, CR3, CR4, properdin, C1Inh, C4bp, MCP, DAF, CD59 (MIRL), clusterin, HRF, and allelic and species variants of any complement polypeptide.

As used herein, the terms “treatment,” “treating,” or “treat” refers to any specific method or procedure used for the cure of, inhibition of, prophylaxis of, reduction of, elimination of, or the amelioration of a disease or pathological condition including, for example, age related macular degeneration, cancer, thrombosis, restenosis, neointimal formation, coronary artery disease, atherosclerosis, wounds, central nervous system injuries, peripheral nervous system injuries, and ischemia.

As used herein, the term “effective amount” refers to a dosage of an agent described herein administered alone or in conjunction with any additional therapeutic agents that are effective and/or sufficient to provide treatment of a disease or pathological condition, such as age related macular degeneration, cancer, thrombosis, restenosis, neointimal formation, coronary artery disease, atherosclerosis, wounds, central nervous system injuries, peripheral nervous system injuries, and ischemia. The effective amount can vary depending on the subject, the disease being treated, and the treatment being affected.

As used herein, the term “therapeutically effective amount” refers to that amount of an agent described herein administered alone and/or in combination with additional therapeutic agents that results in amelioration of symptoms associated with a disease or pathological condition, such as age related macular degeneration, cancer, thrombosis, restenosis, neointimal formation, coronary artery disease, atherosclerosis, wounds, central nervous system injuries, peripheral nervous system injuries, and ischemia.

As used herein, the terms “parenteral administration” and “administered parenterally” refers to modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrasternal injection and infusion.

As used herein, the terms “pharmaceutically or pharmacologically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, general safety and purity standards as required by FDA Office of Biologics standards. Supplementary active ingredients can also be incorporated into the compositions.

As used herein, “Unit dosage” formulations are those containing a dose or sub-dose of the administered ingredient adapted for a particular timed delivery. For example, exemplary “unit dosage” formulations are those containing a daily dose or unit or daily sub-dose or a weekly dose or unit or weekly sub-dose and the like.

Embodiments of this application relate to methods and compositions of modulating growth factor responses of cells, and thereby affecting growth, viability, function, or mitosis of the cells to treat diseases, disorders, and conditions where inhibition or promotion of a growth factor response is desired. The methods can include administering to cells expressing C3a receptor (C3aR) and C5a receptor (C5aR) and at least one growth factor receptor at least one agent that modulates (e.g., inhibits or promotes) C3aR and/or C5aR signaling of the cells.

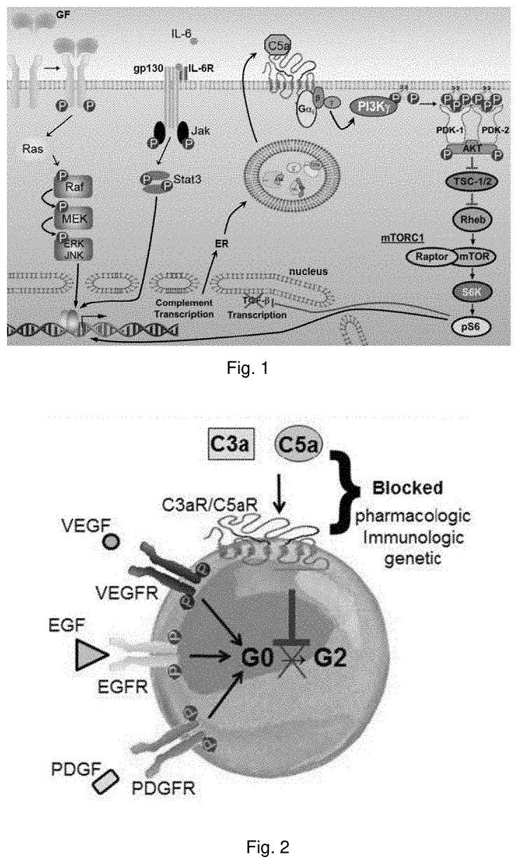

It was found that C3aR/C5aR signaling resulting from C3a/C5a endogenously produced by the same cell plays a central role in the function of many, if not most, receptor tyrosine kinases (RTKs) and some G protein coupled receptors, (GPCRs), affecting viability and cell proliferation, and tissue homeostasis and function (FIG. 1).

Studies of T cell activation during interaction of antigen presenting dendritic cells (DCs) with cognate T cells showed that both partners locally synthesize complement and that paracrine/autocrine interactions of locally produced C3a/C5a with C3aR/C5aR on both partners provide costimulatory and survival signals to the T cells. We found that the intracellular signaling pathways underlying these processes are essential for phosphoinositide-3 kinase γ (PI-3Kγ) activation and consequent inner leaflet phosphatidylinositol 3,4,5 trisphosphate (PtdIns 3,4,5-P₃) generation needed for AKT phosphorylation and downstream signaling to NF-κB. Taken together, the findings indicated that potentiated C3aR/C5aR signaling as contrasted to disabled C3aR/C5aR signaling in T cells (i.e., proliferation vs. PCD) is controlled by surface DAF.

We tested whether this autocrine signaling operates in other cell types and found that this signaling supports the viability of primary cultured ECs, SMCs, embyonic fibroblasts (pMEFs), breast and gastrointestinal epithelial cells (EPCs) as well as more than ten cancer lines of different lineages and that its interruption in all cases induces apoptosis. We found that the mitotic and/or viability effects of seven RTKs and one GPCR depend on autocrine C3aR/C5aR signaling. We also found that IL-6 receptor (IL-6R) and Stat3 are involved in hormone and cytokine growth induction as well as local C5a in the mitotic effects of thrombin.

We also found that RTK signaling interconnects with autocrine C3aR/C5aR signaling and that blockade of either the receptors or their ligands completely abrogated EGF induced growth. Prompted by this result, we performed parallel studies of VEGF-A and PDGF-AA growth induction initially in murine EC lines (bEND.3 and MS-1) and in NIH-3T3 cells, respectively. These experiments surprising yielded near identical results.

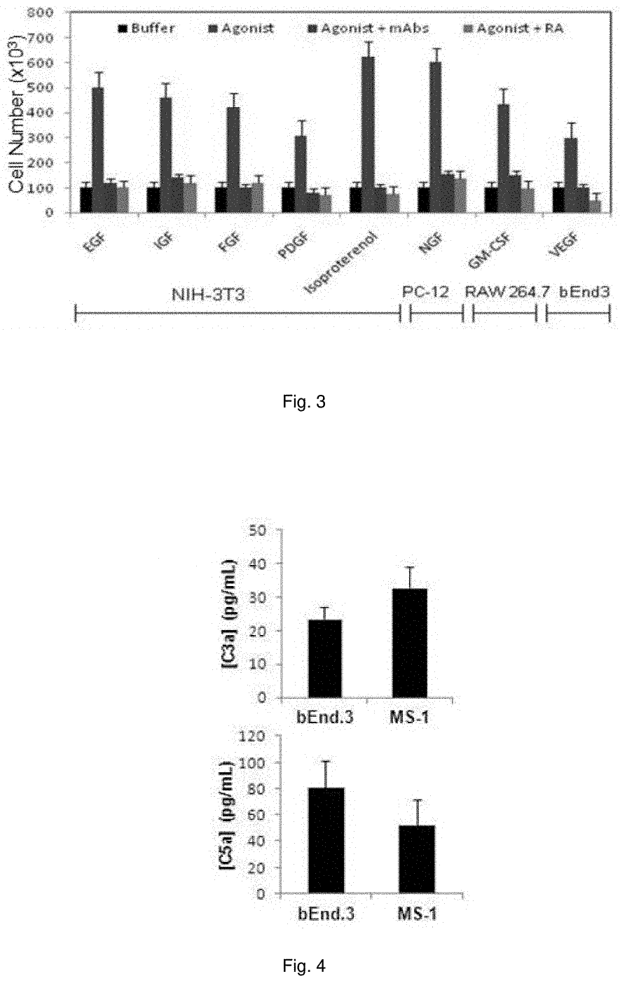

Because this dependence of growth factor responsiveness on C3aR/C5aR signaling could be indirect, i.e., a consequence of its requirement for viability, we performed cell cycle assays. Adding C5a to serum starved NIH-3T3 cells, bEND.3 ECs, or TC-1 cancer cells caused transition from G0 into G2 identically to that of adding EGF to NIH-3T3 or TC-1 cells or adding VEGF-A to bEND.3 ECs (FIGS. 2 and 3). Importantly, including C3aR-A/C5aR-A together with EGF or with VEGF-A markedly blunted or abolished triggering of the cell cycle by EGF and VEGF-A, suggesting that autocrine C3aR/C5aR signals not only limit apoptosis but are needed for cell cycle progression.

To gain mechanistic insight, we focused on VEGF-A growth induction through VEGFR2 in ECs. As a first test of whether VEGFR2 growth induction is interconnected with upregulated C3aR/C5aR signaling, we examined the effect of added VEGF-A on local complement production by the MS-1 and bEND.3 EC cell lines. ELISAs of their culture supernatants showed that VEGF-A increased local C3a as well as C5a production, both ˜8-fold and that both increases were abolished by the inclusion of C3aR-A/C5aR-A. Adding C5a to serum starved HUVEC caused transition from G0 into G2 identically to adding VEGF-A, whereas C3aR/C5aR blockade prevented VEGF-A triggering of the cell cycle. These findings together with the dependence VEGF-A growth induction on C3aR/C5aR signaling indicated that VEGFR2 signals amplify C3aR/C5aR signal transduction and that amplification of this autocrine GPCR signaling integrates with VEGFR2 growth signals. Consistent with the increased local C3a/C5a production, VEGF-A upregulated mRNA transcripts of all of the components/receptors associated with autocrine C3aR/C5aR signaling in primary cultured aortic ECs, whereas antagonizing C3aR/C5aR abrogated the up-regulations and induced markers of apoptosis.

To determine if the linkage between VEGF-A and C3aR/C5aR signaling in ECs involves IL-6, we incubated MS-1 ECs with 1) VEGF-A alone, IL-6 alone, or VEGF-A plus anti-IL-6 mAb, or with 2) IL-6 alone or IL-6 plus C3aR-A/C5aR-A, and assayed cell growth. IL-6 induced EC growth comparably to VEGF-A and VEGF-A's growth induction was abolished by anti-IL-6 mAb. The EC induced growth by IL-6, like that of VEGF-A, was abolished by C3aR-A/C5aR-A. Both VEGF-A and IL-6 induced Stat3 phosphorylation. Importantly, the Stat3 phosphorylation in both cases was abolished by C3aR/C5aR antagonism. Relevant to this, VEGF-A treatment or WT aortic ECs upregulated C3/C5 and increased local C3a/C5a generation as found for MS-1 ECs, but neither change occurred in the presence of anti-IL-6 mAb or the JAK1 inhibitor. These findings thus indicate that VEGFR2 signaling interconnects with C3aR/C5aR signaling via a process involving induction of IL-6 and activation of Stat3.

We surprisingly have found that the thrombin specific inhibitors ANGIOMAX, and antithrombin, as well as other thrombin specific inhibitors PPACK and FM19 virtually abolish T cell activation in response anti-CD3/28 stimulation as well as abolish the proliferation of ECs in response to VEGF-A stimulation. We additionally have found that both T cell activation and VEGF induced EC growth induction are blocked by the MMP-9 and -1 inhibitor. Since some MMPs (MMP-9) is/are constitutively expressed and prothrombin synthesis and secretion might be induced by VEGF-A, prothrombin can cleaved to thrombin by an MMP, e.g., MMP-9 and thrombin, in turn, generates C5a from locally produced C5. The MMP inhibitor alternatively could exert its inhibitory effect on VEGF-A induced EC growth by the pathway that couples C3aR/C5aR signal transduction back to VEGFR2.

Accordingly, based at least in part on these findings, in some embodiments of the application a population of cells expressing C3a receptor (C3aR) and C5a receptor (C5aR) and at least one growth factor receptor (e.g., RTK), such as smooth muscle cells, endothelial cells, leukocytes, cancer cells, neural cells, or fibroblasts, can be contacted (e.g., directly or locally) with a therapeutically effective amount of an agent that modulates (e.g., inhibits or promotes) C3aR and/or C5aR signaling of the cells and modulates (e.g., inhibits or promotes) response of the cells to a growth factor. This modulation of growth factor response can affect viability, function, or mitosis of the cells and treat diseases, disorders, and conditions where inhibition or promotion of a growth factor response is desired.

In some embodiments, a growth factor response (e.g., function, growth, viability, and/or mitosis) of a cell expressing C3a receptor (C3aR) and C5a receptor (C5aR) and at least one growth factor receptor (e.g., RTK), such as smooth muscle cells, endothelial cells, leukocytes, cancer cells, neural cells, or fibroblasts, can be inhibited by administering to the cell an agent that inhibits C3aR and/or C5aR signaling of the cells. The agent can be selected from the group consisting of a complement antagonist that inhibits or substantially reduces the interaction of at least one of C3a or C5a with the C3a receptor (C3aR) and C5a receptor (C5aR), an IL-6/STAT3 signaling pathway antagonist, a thrombin inhibitor and combinations thereof.

By inhibiting or substantially reducing the activity of a complement component, it is meant that the activity of the complement component may be entirely or partly diminished. For example, an inhibition or reduction in the functioning of a C3/C5 convertase may prevent cleavage of C5 and C3 into C5a and C3a, respectively. An inhibition or reduction in the functioning of C5, C3, C5a and/or C3a polypeptides may reduce or eliminate the ability of C5a and C3a to bind C5aR and C3aR, respectively. An inhibition or reduction in Factor B, Factor D, properidin, Bb, Ba and/or any other protein of the complement pathway that is used in the formation of C3 convertase, C5 convertase, C5, C3, C5a and/or C3a may reduce or eliminate the ability of C5a and C3a to be formed and bind to C5aR and C3aR, respectively. Additionally, an inhibition or reduction in the functioning of a C5aR or C3aR may similarly reduce or eliminate the ability of C5a and C3a to bind C5aR and C3aR, respectively.

In an aspect of the application, the at least one complement antagonist can include an antibody or antibody fragment directed against a complement component that can affect or inhibit the formation of C3a and/or C5a (e.g., anti-Factor B, anti-Factor D, anti-C5, anti-C3, anti-C5 convertase, and anti-C3 convertase) and/or reduce C5a/C3a-C5aR/C3aR interactions (e.g., anti-C5a, anti-C3a, anti-C5aR, and C3aR antibodies). In one example, the antibody or antibody fragment can be directed against or specifically bind to an epitope, an antigenic epitope, or an immunogenic epitope of a C5, C3, C3a, C5a, C5aR, C3aR, C5 convertase, and/or C3 convertase. The term “epitope” as used herein can refer to portions of C5, C3, C3a, C5a, C5aR, C3aR, C5 convertase, and/or C3 convertase having antigenic or immunogenic activity. An “immunogenic epitope” as used herein can include a portion of a C5, C3, C3a, C5a, C5aR, C3aR, C5 convertase, and/or C3 convertase that elicits an immune response in a subject, as determined by any method known in the art. The term “antigenic epitope” as used herein can include a portion of a polypeptide to which an antibody can immunospecifically bind as determined by any method well known in the art.

Examples of antibodies directed against C5, C3, C3a, C5a, C5aR, C3aR, C5 convertase, and/or C3 convertase are known in the art. For example, mouse monoclonal antibodies directed against C3aR can include those available from Santa Cruz Biotechnology, Inc. (Santa Cruz, Calif.). Monoclonal anti-human C5aR antibodies can include those available from Research Diagnostics, Inc. (Flanders, N.J.). Monoclonal anti-human/anti-mouse C3a antibodies can include those available from Fitzgerald Industries International, Inc. (Concord, Me.). Monoclonal anti-human/anti-mouse C5a antibodies can include those available from R&D Systems, Inc. (Minneapolis, Minn.).

In some embodiments, the complement antagonist can include purified polypeptide that is a dominant negative or competitive inhibitor of C5, C3, C3a, C5a, C5aR, C3aR, C5 convertase, and/or C3 convertase. As used herein, “dominant negative” or “competitive inhibitor” refers to variant forms of a protein that inhibit the activity of the endogenous, wild type form of the protein (i.e., C5, C3, C3a, C5a, C5aR, C3aR, C5 convertase, and/or C3 convertase). As a result, the dominant negative or competitive inhibitor of a protein promotes the “off” state of protein activity. In the context of the present invention, a dominant negative or competitive inhibitor of C5, C3, C3a, C5a, C5aR, C3aR, C5 convertase, and/or C3 convertase is a C5, C3, C3a, C5a, C5aR, C3aR, C5 convertase, and/or C3 convertase polypeptide, which has been modified (e.g., by mutation of one or more amino acid residues, by posttranscriptional modification, by posttranslational modification) such that the C5, C3, C3a, C5a, C5aR, C3aR, C5 convertase, and/or C3 convertase inhibits the activity of the endogenous C5, C3, C3a, C5a, C5aR, C3aR, C5 convertase, and/or C3 convertase.

In some embodiments, the competitive inhibitor of C5, C3, C3a, C5a, C5aR, C3aR, C5 convertase, and/or C3 convertase can be a purified polypeptide that has an amino acid sequence, which is substantially similar (i.e., at least about 75%, about 80%, about 85%, about 90%, about 95% similar) to the wild type C5, C3, C3a, C5a, C5aR, C3aR, C5 convertase, and/or C3 convertase but with a loss of function. The purified polypeptide, which is a competitive inhibitor of C5, C3, C3a, C5a, C5aR, C3aR, C5 convertase, and/or C3 convertase, can be administered to a cell expressing C5aR and/or C3aR.

It will be appreciated that antibodies directed to other complement components used in the formation of C5, C3, C5a, C3a, C5 convertase, and/or C3 convertase can be used in accordance with the method described herein to reduce and/or inhibit interactions C5a and/or C3a with C5aR and C3aR. The antibodies can include, for example, known Factor B, properdin, and Factor D antibodies that reduce, block, or inhibit the formation of C5a and/or C3a.

In some embodiments, the complement antagonist can include RNA interference (RNAi) polynucleotides to induce knockdown of an mRNA encoding a complement component. For example, an RNAi polynucleotide can comprise a siRNA capable of inducing knockdown of an mRNA encoding a C3, C5, C5aR, or C3aR polypeptide.

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo.

As used herein, the term “dsRNA” refers to siRNA molecules or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response.

As used herein, the term “RNAi construct” is a generic term used throughout the specification to include small interfering RNAs (siRNAs), hairpin RNAs, and other RNA species, which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (I) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences.

The choice of promoter and other regulatory elements generally varies according to the intended host cell. In general, expression vectors of utility in recombinant DNA techniques are often in the form of “plasmids” which refer to circular double stranded DNA loops, which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

Examples of a siRNA molecule directed to an mRNA encoding a C3a, C5a, C5aR, or C3aR polypeptide are known in the art. For instance, human C3a, C3aR, and C5a siRNA is available from Santa Cruz Biotechnology, Inc. (Santa Cruz, Calif.). Additionally, C5aR siRNA is available from Qiagen, Inc. (Valencia, Calif.). siRNAs directed to other complement components, including C3 and C5, are known in the art.

In other embodiments, the RNAi construct can be in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects, which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Such hairpin RNAs are engineered in cells or in an animal to ensure continuous and stable suppression of a desired gene. It is known in the art that siRNAs can be produced by processing a hairpin RNA in the cell.

In yet other embodiments, a plasmid can be used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the a recombinant vector can have the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Exemplary methods of making and delivering either long or short RNAi constructs can be found, for example, in WO01/68836 and WO01/75164.

Examples RNAi constructs that specifically recognize a particular gene or a particular family of genes, can be selected using methodology outlined in detail above with respect to the selection of antisense oligonucleotide. Similarly, methods of delivery RNAi constructs include the methods for delivery antisense oligonucleotides outlined in detail above.

In some embodiments, a lentiviral vector can be used for the long-term expression of a siRNA, such as a short-hairpin RNA (shRNA), to knockdown expression of C5, C3, C5aR, and/or C3aR in cells expressing C3a receptor (C3aR) and C5a receptor (C5aR) and at least one growth factor receptor (e.g., RTK), such as smooth muscle cells, endothelial cells, leukocytes, cancer cells, neural cells, or fibroblasts. Although there have been some safety concerns about the use of lentiviral vectors for gene therapy, self-inactivating lentiviral vectors are considered good candidates for gene therapy as they readily transfect mammalian cells.

It will be appreciated that RNAi constructs directed to other complement components used in the formation of C5, C3, C5a, C3a, C5 convertase, and/or C3 convertase components can be used in accordance with the method described herein to reduce and/or inhibit interactions C5a and/or C3a with C5aR and C3aR on the cells expressing C3a receptor (C3aR) and C5a receptor (C5aR) and at least one growth factor receptor (e.g., RTK), such as smooth muscle cells, endothelial cells, leukocytes, cancer cells, neural cells, or fibroblasts. The RNAi constructs can include, for example, known Factor B, properdin, and Factor D siRNA that reduce expression of Factor B, properdin, and Factor D.

Moreover, it will be appreciated that other antibodies, small molecules, and/or peptides that reduce or inhibit the formation of C5, C3, C5a, C3a, C5 convertase, and/or C3 convertase and/or that reduce or inhibit interactions C5a and/or C3a with C5aR and C3aR on the cells expressing C3a receptor (C3aR) and C5a receptor (C5aR) and at least one growth factor receptor (e.g., RTK) can be used as a complement antagonist in accordance with the method described herein. These other complement antagonists can be administered to the cells expressing C3a receptor (C3aR) and C5a receptor (C5aR) and at least one growth factor receptor (e.g., RTK) at amount effective to inhibit a growth factor response. Example of such other complement antagonists include C5aR antagonists, such as AcPhe[Orn-Pro-D-cyclohexylalanine-Trp-Arg, prednisolone, and infliximab (Woodruff et al., The Journal of Immunology, 2003, 171: 5514-5520), hexapeptide MeFKPdChaWr (March et al., Mol Pharmacol 65:868-879, 2004), PMX53 and PMX205, and N-[(4-dimethylaminophenyl)methyl]-N-(4-isopropylphenyl)-7-methoxy-1,2,3,4-tetrahydronaphthalen-1-carboxamide hydrochloride (W-54011) (Sumichika et al., J. Biol. Chem., Vol. 277, Issue 51, 49403-49407, Dec. 20, 2002), and a C3aR antagonist, such as SB 290157 (Ratajczak et al., Blood, 15 Mar. 2004, Vol. 103, No. 6, pp. 2071-2078).

In other embodiments, the agent that inhibits C3aR and/or C5aR signaling in a cell expressing C3a receptor (C3aR) and C5a receptor (C5aR) and at least one growth factor receptor (e.g., RTK), such as smooth muscle cells, endothelial cells, leukocytes, cancer cells, neural cells, or fibroblasts, can include an IL-6/STAT3 signaling pathway antagonist that substantially decreases or inhibits the expression and/or functional activity of a component of the IL-6/STAT3 signaling pathway in the cell. The functional activity of the IL-6/STAT3 signaling pathway can be suppressed, inhibited, and/or blocked in several ways including: direct inhibition of the activity of IL-6 and/or STAT3 (e.g., by using neutralizing antibodies, small molecules or peptidomimetics, dominant negative polypeptides); inhibition of genes that express IL-6 and/or STAT-3 (e.g., by blocking the expression or activity of the genes and/or proteins); activation of genes and/or proteins that inhibit one or more of the functional activity of IL-6 and/or STAT3 (e.g., by increasing the expression or activity of the genes and/or proteins); inhibition of genes and/or proteins that are downstream mediators of the iNOS expression (e.g., by blocking the expression and/or activity of the mediator genes and/or proteins); introduction of genes and/or proteins that negatively regulate one or more of functional activity of IL-6 and/or STAT3 (e.g., by using recombinant gene expression vectors, recombinant viral vectors or recombinant polypeptides); or gene replacement with, for instance, a hypomorphic mutant of STAT-3 (e.g., by homologous recombination, overexpression using recombinant gene expression or viral vectors, or mutagenesis).

In an embodiment of the application, the IL-6/STAT3 signaling pathway antagonist is an IL-6 antagonist. In some aspects, the IL-6 antagonist can include a humanized IL-6 receptor-inhibiting monoclonal antibody. In certain aspects, the IL-6 antagonist is the product tocilizumab (a descriptive name sold under the trademark ACTEMRA by Roche, Switzerland). In other aspects, the IL-6 antagonist can include a vaccine that when administered to a subject generates IL-6 antibodies in the subject. An example of such a vaccine is disclosed in Fosergau et al. Journal of Endocrinology (2010) 204, 265-273.

In another embodiment, the IL-6/STAT3 signaling pathway antagonist is a tyrosine kinase inhibitor. Exemplary tyrosine kinase inhibitors for use in the present invention include but are not limited to tyrphostins, in particular AG-490, and inhibitors of Jak, Src, and BCR-Abl tyrosine kinases. Other tyrphostins suitable for use herein include, but are not limited to AG17, AG213 (RGS0864), AG18, AG82, AG494, AG825, AG879, AG1112, AG1296, AG1478, AG126, RG13022, RG14620, AG555, and related compounds. In certain aspects, a BCR-Abl tyrosine kinase inhibitor for use herein can include the product imatinib mesilate (a descriptive name sold under the trademark GLEEVEC® by Novartis, Switzerland).

In a further embodiment, the IL-6/STAT3 signaling pathway antagonist is an HMG CoA reductase inhibitor (3-hydroxymethylglutaryl coenzyme A reductase inhibitors) (e.g., statin). HMG-CoA (3-hydroxy methylglutaryl coenzyme A) reductase is the microsomal enzyme that catalyzes the rate limiting reaction in cholesterol biosynthesis (HMG-CoA Mevalonate.

Statins that can be used for administration, or co-administration with other agents described herein include, but are not limited to, simvastatin (U.S. Pat. No. 4,444,784), mevistatin, lovastatin (U.S. Pat. No. 4,231,938), pravastatin sodium (U.S. Pat. No. 4,346,227), fluvastatin (U.S. Pat. No. 4,739,073), atorvastatin (U.S. Pat. No. 5.273,995), cerivastatin, and numerous others described in U.S. Pat. Nos. 5,622,985, 5,135,935, 5,356,896, 4,920,109, 5,286,895, 5,262,435, 5,260,332, 5,317,031, 5,283,256, 5,256,689, 5,182,298, 5,369,125, 5,302,604, 5,166,171, 5,202,327, 5,276,021, 5,196,440, 5,091,386, 5,091,378, 4,904,646, 5,385,932, 5,250,435, 5,132,312, 5,130,306, 5,116,870, 5,112,857, 5,102,911, 5,098,931, 5,081,136, 5,025,000, 5,021,453, 5,017,716, 5,001,144, 5,001,128, 4,997,837, 4,996,234, 4,994,494, 4,992,429, 4,970,231, 4,968,693, 4,963,538, 4,957,940, 4,950,675, 4,946,864, 4,946,860 4,940,800, 4,940,727, 4,939,143, 4,929,620, 4,923,861, 4,906,657, 4,906,624 and 4,897,402, the disclosures of which patents are incorporated herein by reference.

In yet another embodiment, the IL-6/STAT3 signaling pathway antagonist can be a STAT3 inhibitor. Examples of STAT3 inhibitors are described in U.S. Patent Application No. 2010/0041685 and can include 4-[3-(2,3-dihydro-1,4-benzodioxin-6-yl)-3-oxo-1-propen-1-yl]benzoic acid; 4{5-[(3-ethyl-4-oxo-2-thioxo-1,3-thiazolidin-5-ylidene)methyl]-2-furyl}benzoic acid; 4-[({3-[(carboxymethyl)thio]-4-hydroxy-1-naphthyl}amino)sulfonyl]benzoic acid; 3-({2-chloro-4-[(1,3-dioxo-1,3-dihydro-2H-inden-2-ylidene)methyl]-6-ethoxyphenoxy}methyl)benzoic acid; methyl 4-({[3-(2-methyoxy-2-oxoethyl)-4,8-dimethyl-2-oxo-2H-chromen-7-yl]oxy}methyl)benzoate; 4-chloro-3-{5-[(1,3-diethyl-4,6-dioxo-2-thioxotetrahydro-5(2H)-pyrimidinylidene)methyl]-2-furyl}benzoic acid; a functionally active derivative thereof and a mixture thereof. Other examples of STAT3 inhibitors are described in WO 2010/118309 and in G. Zinzalla et al. Bioorg. Med. Chem. Lett. 20 (2010)7029-7032.

In other embodiments, the agent that inhibits C3aR and/or C5aR signaling in a expressing C3a receptor (C3aR) and C5a receptor (C5aR) and at least one growth factor receptor (e.g., RTK), such as smooth muscle cells, endothelial cells, leukocytes, cancer cells, neural cells, or fibroblasts, can include thrombin inhibitor that substantially decreases or inhibits thrombin cleavage of C3 and/or C5 generated by the cell to C3a and C5a that can bind to C3aR and C5aR.

Thrombin inhibitors that can used in the methods described herein include those which inhibit thrombosis, including but not limited to those described in U.S. Pat. Nos. 5,536,708, 5,510,369, 5,672,582, 5,714,485, 5,629,324 (e.g. N′-[[1-(Aminoiminomethyl)-4-piperidinyl]methyl]-N-(3,3-diphenylpropionyl)-L-proline amide), U.S. Pat. No. 5,668,289 (e.g., 3-(2-Phenethylamino)-6-methyl-1-(2-amino-6-methyl-5-methylenecarboxamidomethylpyridinyl)-2-pyridinone), U.S. Pat. Nos. 5,744,486, 5,798,377, WO 9631504, WO9611941, WO9606832, WO9606849, WO9420467, WO 9632110, U.S. Pat. No. 4,496,653, WO 9715190, and WO 9740024, e.g., 3-(2-Phenylethylamino)-6-methyl-1-(2-amino-6-methyl-5-methylene-carboxamidomethylpyridinyl)-2-pyrazinone, the contents of which are hereby incorporated by reference.

Other examples of thrombin inhibitors include low molecular weight peptide-based thrombin inhibitors. The term “low molecular weight peptide-based thrombin inhibitors” will be well understood by one skilled in the art to include thrombin inhibitors with one to four peptide linkages, and/or with a molecular weight below 1000, and includes those described in the review paper by Claesson in Blood Coagul. Fibrin. (1994) 5:411, as well as those disclosed in U.S. Pat. No. 4,346,078; International Patent Applications WO 93/11152, WO 95/23609, WO 95/35309, WO 96/25426, WO 94/29336, WO 93/18060 and WO 95/01168; and European Patent Nos. 648 780, 468 231, 559 046, 641 779, 185 390, 526 877, 542 525, 195 212, 362 002, 364 344, 530 167, 293 881, 686 642, 669 317 and 601 459. In some embodiments, the thrombin inhibitor can be selected from the group consisting of ANGIOMAX, PPACK, and FM19.

The at least one agent that inhibits C3aR and/or C5aR signaling can be administered to the cells in vivo or in vitro to inhibit a growth factor response of the cells. The cell can be derived from a human subject, from a known cell line, or from some other source. Examples of cells expressing C3a receptor (C3aR) and C5a receptor (C5aR) and at least one growth factor receptor (e.g., RTK) include smooth muscle cells, endothelial cells, leukocytes, cancer cells, neural cells, or fibroblasts that are located in, for example, in tissue of a human subject. The cell may be isolated or, alternatively, associated with any number of identical, similar, or different cell types.

In some embodiments, the agent that inhibits at least one of C3aR and/or C5aR signaling in the cell expressing C3aR and C5aR may be used to treat animals and patients with aberrant angiogenesis resulting from or mediated by growth factors, such as VEGF. Such aberrant angiogenesis, outside the field of cancer treatment, can include or be associated with arthritis, rheumatoid arthritis, psoriasis, atherosclerosis, diabetic retinopathy, age-related macular degeneration, Grave's disease, vascular restenosis, including restenosis following angioplasty, arteriovenous malformations (AVM), meningioma, hemangioma and neovascular glaucoma. Other potential targets for intervention include angiofibroma, atherosclerotic plaques, corneal graft neovascularization, hemophilic joints, hypertrophic scars, osler-weber syndrome, pyogenic granuloma retrolental fibroplasia, scleroderma, trachoma, vascular adhesions, synovitis, dermatitis, various other inflammatory diseases and disorders, and even endometriosis. Further diseases and disorders that are treatable by the compositions described herein, and the unifying basis of such angiogenic disorders, are set forth below.

One disease in which angiogenesis is involved is rheumatoid arthritis, wherein the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Factors associated with angiogenesis also have a role in osteoarthritis, contributing to the destruction of the joint.

Another example of a disease mediated by angiogenesis is ocular neovascular disease. This disease is characterized by invasion of new blood vessels into the structures of the eye, such as the choroid, retina, or cornea. It is the most common cause of blindness and is involved in approximately twenty eye diseases. In age-related macular degeneration, the associated visual problems are caused by an ingrowth of chorioidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma and retrolental fibroplasia.

Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical bums, bacterial ulcers, fungal ulcers, Herpes simplex infections, Herpes zoster infections, protozoan infections, Kaposi sarcoma, Mooren ulcer, Terrien's marginal degeneration, mariginal keratolysis, rheumatoid arthritis, systemic lupus, polyarteritis, trauma, Wegeners sarcoidosis, Scleritis, Steven's Johnson disease, periphigoid radial keratotomy, and corneal graph rejection.

Diseases associated with retinal/choroidal neovascularization include, but are not limited to, diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosis, retinopathy of prematurity, Eales disease, Bechets disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, trauma and post-laser complications.

Other diseases include, but are not limited to, diseases associated with rubeosis and diseases caused by the abnormal proliferation of fibrovascular or fibrous tissue including all forms of proliferative vitreoretinopathy.

Chronic inflammation also involves pathological angiogenesis. Such disease states as ulcerative colitis and Crohn's disease show histological changes with the ingrowth of new blood vessels into the inflamed tissues. Bartonellosis, a bacterial infection found in South America, can result in a chronic stage that is characterized by proliferation of vascular endothelial cells.

Another pathological role associated with angiogenesis is found in atherosclerosis. The plaques formed within the lumen of blood vessels have been shown to have angiogenic stipulatory activity. VEGF expression in human coronary atherosclerotic lesions has been demonstrated. This evidences the pathophysiological significance of VEGF in the progression of human coronary atherosclerosis, as well as in recanalization processes in obstructive coronary diseases. The compositions and methods of this application therefore provide an effective treatment for such conditions.

One of the most frequent angiogenic diseases of childhood is the hemangioma. In most cases, the tumors are benign and regress without intervention. In more severe cases, the tumors progress to large cavernous and infiltrative forms and create clinical complications. Systemic forms of hemangiomas, the hemangiomatoses, have a high mortality rate.

Therapy-resistant hemangiomas exist that cannot be treated with therapeutics currently in use.

Angiogenesis is also responsible for damage found in hereditary diseases such as Osler-Weber-Rendu disease, or hereditary hemorrhagic telangiectasia. This is an inherited disease characterized by multiple small angiomas, tumors of blood or lymph vessels. The angiomas are found in the skin and mucous membranes, often accompanied by epistaxis (nosebleeds) or gastrointestinal bleeding and sometimes with pulmonary or hepatic arteriovenous fistula.

Angiogenesis is also involved in normal physiological processes such as reproduction and wound healing. Angiogenesis is an important step in ovulation and also in implantation of the blastula after fertilization. Prevention of angiogenesis could be used to induce amenorrhea, to block ovulation or to prevent implantation by the blastula.

In wound healing, excessive repair or fibroplasia can be a detrimental side effect of surgical procedures and may be caused or exacerbated by angiogenesis. Adhesions are a frequent complication of surgery and lead to problems such as small bowel obstruction.

Diseases and disorders characterized by undesirable vascular permeability can also be treated by the present invention. These include edema associated with brain tumors, ascites associated with malignancies, Meigs' syndrome, lung inflammation, nephrotic syndrome, pericardial effusion and pleural effusion, as disclosed in WO 98/16551, specifically incorporated herein by reference.

Each of the foregoing diseases and disorders, along with all types of tumors, as described in the following sections, can be effectively treated by the agents described herein, as disclosed in, e.g., U.S. Pat. No. 5,712,291 (specifically incorporated herein by reference), that unified benefits result from the application of anti-angiogenic strategies to the treatment of angiogenic diseases.

The agent that inhibits at least one of C3aR and/or C5aR signaling in the cell expressing C3aR and C5aR can also be utilized in the treatment of tumors. Tumors in which angiogenesis is important include malignant tumors, and benign tumors, such as acoustic neuroma, neurofibroma, trachoma and pyogenic granulomas. Angiogenesis is particularly prominent in solid tumor formation and metastasis. However, angiogenesis is also associated with blood-born tumors, such as leukemias, and various acute or chronic neoplastic diseases of the bone marrow in which unrestrained proliferation of white blood cells occurs, usually accompanied by anemia, impaired blood clotting, and enlargement of the lymph nodes, liver, and spleen. Angiogenesis also plays a role in the abnormalities in the bone marrow that give rise to leukemia-like tumors.

Angiogenesis is important in two stages of tumor metastasis. In the vascularization of the primary tumor, angiogenesis allows cells to enter the blood stream and to circulate throughout the body. After tumor cells have left the primary site, and have settled into the secondary, metastasis site, angiogenesis must occur before the new tumor can grow and expand. Therefore, prevention of angiogenesis can prevent metastasis of tumors and contain the neoplastic growth at the primary site, allowing treatment by other therapeutics, particularly, therapeutic agent-targeting agent constructs.

The agent that inhibits at least one of C3aR and/or C5aR signaling in the cell expressing C3aR and C5aR is broadly applicable to the treatment of any tumor or cancer having a vascular component as well as any tumor or cancer cell that expresses C3aR and/or C5aR. In using the agent that inhibits at least one of C3aR and/or C5aR signaling in the treatment of tumors, particularly vascularized, malignant tumors, the agents may be used alone or in combination with, e.g., chemotherapeutic, radiotherapeutic, apoptopic, anti-angiogenic agents and/or immunotoxins or coaguligands.

Typical vascularized tumors for treatment are the solid tumors, particularly carcinomas, which require a vascular component for the provision of oxygen and nutrients. Exemplary solid tumors that may be treated using the invention include, but are not limited to, carcinomas of the lung, breast, ovary, stomach, pancreas, larynx, esophagus, testes, liver, parotid, biliary tract, colon, rectum, cervix, uterus, endometrium, kidney, bladder, prostate, thyroid, squamous cell carcinomas, adenocarcinomas, small cell carcinomas, melanomas, gliomas, glioblastomas, neuroblastomas, and the like. WO 98/45331 is also incorporated herein by reference to further exemplify the variety of tumor types that may be effectively treated using an agent that inhibits at least one of C3aR and/or C5aR signaling.

Agents that inhibit at least one of C3aR and/or C5aR signaling in the cells expressing C3aR and C5aR can be use in the treatment of any patient that presents with a solid tumor. In light of the specific properties of the agents described herein, the agents will have reduced side effects. Particular advantages will result in the maintenance or enhancement of host immune responses against the tumor, as mediated by macrophages, and in the lack of adverse effects on bone tissue. The agents can be the anti-angiogenic therapy of choice for the treatment of pediatric cancers and patients having, or at risk for developing, osteoporosis and other bone deficiencies.

The agents that inhibit at least one of C3aR and/or C5aR signaling are also intended for use in preventative or prophylactic treatments. These aspects include the ability to treat patients presenting with a primary tumor who may have metastatic tumors, or tumor cells in the earlier stages of metastatic tumor seeding. As an anti-angiogenic strategy, the agents may also be used to prevent tumor development in subjects at moderate or high risk for developing a tumor, as based upon prognostic tests and/or close relatives suffering from a hereditary cancer.

The foregoing anti-angiogenic treatment methods and uses will generally involve the administration of the pharmaceutically effective composition comprising to the animal or patient by any route of administration that allows the agent to localize to the angiogenic site or sites. Such administration routes can include direct administration, including tumor or intratumoral vascular endothelial cells, will be acceptable. Therefore, other suitable routes of delivery include oral, rectal, nasal, topical, and vaginal. U.S. Pat. No. 5,712,291, is specifically incorporated herein by reference for purposes including further describing the various routes of administration that may be included in connection with the treatment of an angiogenic disease or disorder. For conditions associated with the eye, ophthalmic formulations and administration are contemplated.

“Administration”, as used herein, means provision or delivery of the agents that inhibit at least one of C3aR and/or C5aR signaling in an amount(s) and for a period of time(s) effective to exert anti-angiogenic and/or anti-tumor effects.

Therapeutically effective doses of the agents that inhibit at least one of C3aR and/or C5aR signaling are readily determinable using data from an animal model. Experimental animals bearing solid tumors are frequently used to optimize appropriate therapeutic doses prior to translating to a clinical environment. Such models are known to be very reliable in predicting effective anti-cancer strategies. For example, mice bearing solid tumors are widely used in pre-clinical testing.

In using the agents that inhibit at least one of C3aR and/or C5aR signaling in anti-angiogenic therapies, one can also draw on other published data in order to assist in the formulation of doses for clinical treatment. For instance, although the agents and methods of the present invention have distinct advantages over those in the art, the information in the literature concerning treatment with other polypeptides and tyrosine kinase inhibitors can still be used in combination with the data and teaching in the present application to design and/or optimize treatment protocols and doses.

Any dose, or combined medicament of the agents that inhibit at least one of C3aR and/or C5aR signaling, that results in any consistently detectable anti-angiogenic effect, inhibition of metastasis, tumor vasculature destruction, tumor thrombosis, necrosis and/or general anti-tumor effect will define a useful invention. The present invention may also be effective against vessels downstream of the tumor, i.e., target at least a sub-set of the draining vessels, particularly as cytokines released from the tumor will be acting on these vessels, changing their antigenic profile.

It will also be understood that even in such circumstances where the anti-angiogenic and/or tumor effects of the dose, or combined therapy of the agents that inhibit at least one of C3aR and/or C5aR signaling, are towards the low end of the intended therapeutic range, it may be that this therapy is still equally or even more effective than all other known therapies in the context of the particular tumor target or patient. It is unfortunately evident to a clinician that certain tumors and conditions cannot be effectively treated in the intermediate or long term, but that does not negate the usefulness of the present therapy, particularly where it is at least about as effective as the other strategies generally proposed.

In designing appropriate doses of the agents that inhibit at least one of C3aR and/or C5aR signaling for the treatment of vascularized tumors, one may readily extrapolate from the knowledge in the literature in order to arrive at appropriate doses for clinical administration. To achieve a conversion from animal to human doses, one would account for the mass of the agents administered per unit mass of the experimental animal and, preferably, account for the differences in the body surface area (m2) between the experimental animal and the human patient. All such calculations are well known and routine to those of ordinary skill in the art.

It will be understood that lower doses may be more appropriate in combination with other agents, and that high doses can still be tolerated.

Formulation of pharmaceutical compounds for use in the modes of administration noted above (and others) are described, for example, in Remington's Pharmaceutical Sciences (18^{th} edition), ed. A. Gennaro, 1990, Mack Publishing Company, Easton, Pa. (also see, e.g., M. J. Rathbone, ed., Oral Mucosal Drug Delivery, Drugs and the Pharmaceutical Sciences Series, Marcel Dekker, Inc., N.Y., U.S.A., 1996; M. J. Rathbone et al., eds., Modified-Release Drug Delivery Technology, Drugs and the Pharmaceutical Sciences Series, Marcel Dekker, Inc., N.Y., U.S.A., 2003; Ghosh et al., eds., Drug Delivery to the Oral Cavity, Drugs and the Pharmaceutical Sciences Series, Marcel Dekker, Inc., N.Y. U.S.A., 1999.

In one example, the agents that inhibit at least one of C3aR and/or C5aR signaling can be provided in ophthalmic preparation that can be administered to the subject's cornea or eye. The ophthalmic preparation can contain the agents that inhibit at least one of C3aR and/or C5aR signaling in a pharmaceutically acceptable solution, suspension or ointment. Some variations in concentration will necessarily occur, depending on the particular complement antagonist employed, the condition of the subject to be treated and the like, and the person responsible for treatment will determine the most suitable concentration for the individual subject. The ophthalmic preparation can be in the form of a sterile aqueous solution containing, if desired, additional ingredients, for example, preservatives, buffers, tonicity agents, antioxidants, stabilizers, nonionic wetting or clarifying agents, and viscosity increasing agents.

The agents that inhibit at least one of C3aR and/or C5aR signaling can also be formulated for topical administration through the skin. “Topical delivery systems” also include transdermal patches containing the ingredient to be administered. Delivery through the skin can further be achieved by iontophoresis or electrotransport, if desired.

Formulations for topical administration to the skin include, for example, ointments, creams, gels and pastes comprising the complement antagonist in a pharmaceutical acceptable carrier. The formulation of complement antagonists for topical use includes the preparation of oleaginous or water-soluble ointment bases, as is well known to those in the art. For example, these formulations may include vegetable oils, animal fats, and, for example, semisolid hydrocarbons obtained from petroleum. Particular components used may include white ointment, yellow ointment, cetyl esters wax, oleic acid, olive oil, paraffin, petrolatum, white petrolatum, spermaceti, starch glycerite, white wax, yellow wax, lanolin, anhydrous lanolin and glyceryl monostearate. Various water-soluble ointment bases may also be used, including glycol ethers and derivatives, polyethylene glycols, polyoxyl 40 stearate and polysorbates.

In some embodiments, the agent being selected from the group consisting of a STAT3 IL-6 signaling pathway antagonist, a thrombin inhibitor and a combination thereof. The agent can be administered to the cells of the tumor or aberrant angiogenic tissue at amount effective to inhibit at least one of growth, viability, or mitosis of the cells.

In other embodiments, the agent that inhibits at least one of C3aR and/or C5aR signaling in the cell expressing C3aR and C5aR may be used to treat animals and patients with vascular diseases that cause obstruction of the vascular system as result of growth factor responses, such as PDGF. Such diseases can, for example, result from neointimal accumulation on a vascular surface as a result of a vascular injury and PDGF generation. Representative examples of such diseases include artherosclerosis or atherogenesis of all vessels (around any artery, vein or graft) including, but not restricted to: the coronary arteries, aorta, iliac arteries, carotid arteries, common femoral arteries, superficial femoral arteries, popliteal arteries, and at the site of graft anastomosis; vasospasms (for example, coronary vasospasms and Raynaud's Disease); restenosis (obstruction of a vessel at the site of a previous intervention such as balloon angioplasty, bypass surgery, stent insertion and graft insertion); thrombosis inflammatory and autoimmune conditions (for example, Temporal Arteritis, vasculitis).

Briefly, in vascular diseases such as atherosclerosis, white cells, specifically monocytes and T lymphocytes adhere to endothelial cells, especially at locations of arterial branching in response to PDGF signalling. After adhering to the endothelium, leukocytes migrate across the endothelial cell lining in response to chemostatic stimuli, and accumulate in the intima of the arterial wall, along with smooth muscle cells. This initial lesion of athersosclerosis development is known as the “fatty streak”. Monocytes within the fatty streak differentiate into macrophages; and the macrophages and smooth muscle cells progressively take up lipids and lipoprotein to become foam cells.

As macrophages accumulate, the overlying endothelium becomes mechanically disrupted and chemically altered by oxidized lipid, oxygen-derived free radicals and proteases which are released by macrophages. Foam cells erode through the endothelial surface causing micro-ulcerations of the vascular wall. Exposure of potentially thrombogenic subendothelial tissues (such as collagen and other proteins) to components of the bloodstream results in adherence of platelets to regions of disrupted endothelium. Platelet adherence and other events triggers the elaboration and release of growth factors into this milieu, including platelet-derived growth factor (PDGF), platelet activating factor (PAF), and interleukins 1 and 6 (IL-1, IL-6). These paracrine factors are thought to stimulate vascular smooth muscle cell (VSMC) migration and proliferation.

In addition to PDGF, IL-1 and IL-6, other mitogenic factors are produced by cells which infiltrate the vessel wall including: transforming growth factor β (TGF-β), fibroblast growth factor (FGF), thrombospondin, serotonin, thromboxane A₂, norepinephrine, and angiotensin II. This results in the recruitment of more cells, elaboration of further extracellular matrix and the accumulation of additional lipid. This progressively enlarges the atherosclerotic lesion until it significantly encroaches upon the vascular lumen. Initially, obstructed blood flow through the vascular tube causes ischemia of the tissues distal to the atherosclerotic plaque only when increased flow is required--later as the lesion further blocks the artery, ischemia occurs at rest.

Macrophages in the enlarging atherosclerotic plaque release oxidized lipid, free radicals, elastases, and collagenases that cause cell injury and necrosis of neighbouring tissues. The lesion develops a necrotic core and is transformed into a complex plaque. Complex plaques are unstable lesions that can: break off causing embolization; local hemorrhage (secondary to rupture of the vasa vasora supplying the plaque which results in lumen obstruction due to rapid expansion of the lesion); or ulceration and fissure formation (this exposes the thrombogenic necrotic core to the blood stream producing local thrombosis or distal embolization). Even should none of the above sequel occur, the adherent thrombus may become organized and incorporated into the plaque, thereby accelerating its growth. Furthermore, as the local concentrations of fibrinogen and thrombin increase, proliferation of vascular smooth muscle cells within the media and intima is stimulated; a process which also ultimately leads to additional narrowing of the vessel.

Agents that inhibit C3aR and/or C5aR signaling can be administered to cells expressing C3aR and/or C5aR, such as vascular endothelial cells, smooth muscle cells, and macrophages, to inhibit PDGF mediated accumulation of plaque. In some embodiments, the agent can be administered directly to the cells using, for example, a medical device that can be delivered to the vasculature.

The medical device can include, for example, endovascular medical devices, such as intracoronary medical devices. Examples of intracoronary medical devices can include stents, drug delivery catheters, grafts, and drug delivery balloons utilized in the vasculature of a subject. Where the medical device comprises a stent, the stent may include peripheral stents, peripheral coronary stents, degradable coronary stents, non-degradable coronary stents, self-expanding stents, balloon-expanded stents, and esophageal stents. The medical device may also include arterio-venous grafts, by-pass grafts, penile implants, vascular implants and grafts, intravenous catheters, small diameter grafts, artificial lung catheters, electrophysiology catheters, bone pins, suture anchors, blood pressure and stent graft catheters, breast implants, benign prostatic hyperplasia and prostate cancer implants, bone repair/augmentation devices, breast implants, orthopedic joint implants, dental implants, implanted drug infusion tubes, oncological implants, pain management implants, neurological catheters, central venous access catheters, catheter cuff, vascular access catheters, urological catheters/implants, atherectomy catheters, clot extraction catheters, PTA catheters, PTCA catheters, stylets (vascular and non-vascular), drug infusion catheters, angiographic catheters, hemodialysis catheters, neurovascular balloon catheters, thoracic cavity suction drainage catheters, electrophysiology catheters, stroke therapy catheters, abscess drainage catheters, biliary drainage products, dialysis catheters, central venous access catheters, and parental feeding catheters.

In some embodiments, the agent can include a complement antagonist that is coated or provided on an endovascular device and is used to treat a vascular injury. The vascular injury can include, for example, atherosclerosis, thrombosis, stenosis, restenosis, and/or neoinitimal accumulation that results from vascular injury. The complement antagonist can include DAF or an antibody directed against at least one of C3, C5, C3 convertase, C5 convertase, C3a, C5a, C3aR, or C5aR. The complement antagonist can be administered at amount effective to inhibit neointimal accumulation and/or stenosis of the vasculature.

In other embodiments of the application, a growth factor response (e.g., function, growth, viability, and/or mitosis) of a cell expressing C3a receptor (C3aR) and C5a receptor (C5aR) and at least one growth factor receptor (e.g., RTK), such as smooth muscle cells, endothelial cells, leukocytes, cancer cells, neural cells, or fibroblasts, can be promoted or stimulated by administering to the cell an agent that promotes or stimulates C3aR and/or C5aR signaling of the cells. The agent can be selected from the group consisting of C3, C5, C3a, C5a, a C3aR agonist, a C5aR agonist, a DAF antagonist, or combination thereof.

Promotion or stimulation of C3aR and/or C5aR activation in cells expressing C3a receptor (C3aR) and C5a receptor (C5aR) and at least one growth factor receptor (e.g., RTK), such as smooth muscle cells, endothelial cells, neural cells, or fibroblasts induce a growth factor response in the cells can be used to stimulate, promote, and/or enhance growth, viability, function, or mitosis of the cells in response to growth factor stimulation.

In some embodiments, an agent that promotes or stimulates C3aR and/or C5aR signaling can be used to treat ischemic disorders and/or tissue injury in a mammalian subject. The ischemic disorder and/or tissue injury can comprise, for example, a peripheral vascular disorder, a pulmonary embolus, a venous thrombosis, a myocardial infarction, a transient ischemic attack, unstable angina, cerebral vascular ischemia, a reversible ischemic neurological deficit, ischemic kidney disease, or a stroke disorder.

The ischemic disorder can also comprise an iatrogenically induced ischemic disorder. The iatrogenic ischemic disorder can result from a subject undergoing, for example, angioplasty, heart surgery, lung surgery, spinal surgery, brain surgery, vascular surgery, abdominal surgery, kidney surgery, or organ transplantation surgery. The organ transplantation can comprise heart, lung, pancreas, kidney, or liver translation surgery.

The agent that promotes or stimulates C3aR and/or C5aR signaling of the cells can be administered directly to or about the periphery of ischemic tissue in order to promote or stimulate angiogenesis of the ischemic tissue. In one aspect of the invention, the agent that promotes or stimulates C3aR and/or C5aR signaling of the cells can be delivered to or about the periphery of the ischemic tissue by administering the agent neat or in a pharmaceutical composition to or about the ischemic tissue. The pharmaceutical composition can provide localized release of the agent to the ischemic tissue or cells being treated. Pharmaceutical compositions in accordance with the invention will generally include an amount of the agent that promotes or stimulates C3aR and/or C5aR signaling of the cells or variants thereof admixed with an acceptable pharmaceutical diluent or excipient, such as a sterile aqueous solution, to give a range of final concentrations, depending on the intended use. The techniques of preparation are generally well known in the art as exemplified by Remington's Pharmaceutical Sciences, 16th Ed. Mack Publishing Company, 1980, incorporated herein by reference. Moreover, for human administration, preparations should meet sterility, pyrogenicity, general safety and purity standards as required by FDA Office of Biological Standards.

The pharmaceutical composition can be in a unit dosage injectable form (e.g., solution, suspension, and/or emulsion). Examples of pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (e.g., glycerol, propylene glycol, liquid polyethylene glycol, and the like), suitable mixtures thereof and vegetable oils.

Proper fluidity can be maintained, for example, by the use of a coating, such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various amounts of the other ingredients, as desired.

Pharmaceutical “slow release” capsules or “sustained release” compositions or preparations may be used and are generally applicable. Slow release formulations are generally designed to give a constant drug level over an extended period and may be used to deliver the agent. The slow release formulations are typically implanted in the vicinity of the ischemic tissue site, for example, at the site of a cell expressing C3aR and/or C5aR in or about the ischemic tissue.

Examples of sustained-release preparations include semipermeable matrices of solid hydrophobic polymers containing the agent that promotes or stimulates C3aR and/or C5aR signaling of the cells, which matrices are in the form of shaped articles, e.g., films or microcapsule. Examples of sustained-release matrices include polyesters; hydrogels, for example, poly(2-hydroxyethyl-methacrylate) or poly(vinylalcohol); polylactides, e.g., U.S. Pat. No. 3,773,919; copolymers of L-glutamic acid and y ethyl-L-glutamate; non-degradable ethylene-vinyl acetate; degradable lactic acid-glycolic acid copolymers, such as the LUPRON DEPOT (injectable microspheres composed of lactic acid-glycolic acid copolymer and leuprolide acetate); and poly-D-(−)-3-hydroxybutyric acid.

While polymers such as ethylene-vinyl acetate and lactic acid-glycolic acid enable release of molecules for over 100 days, certain hydrogels release proteins for shorter time periods. When encapsulated agent remain in the body for a long time, and may denature or aggregate as a result of exposure to moisture at 37° C., thus reducing biological activity and/or changing immunogenicity. Rational strategies are available for stabilization depending on the mechanism involved. For example, if the aggregation mechanism involves intermolecular S—S bond formation through thio-disulfide interchange, stabilization is achieved by modifying sulfhydryl residues, lyophilizing from acidic solutions, controlling moisture content, using appropriate additives, developing specific polymer matrix compositions, and the like.

In certain embodiments, liposomes and/or nanoparticles may also be employed with the agent that promotes or stimulates C3aR and/or C5aR signaling of the cells. The formation and use of liposomes is generally known to those of skill in the art, as summarized below.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 \acute{Å}, containing an aqueous solution in the core.

Phospholipids can form a variety of structures other than liposomes when dispersed in water, depending on the molar ratio of lipid to water. At low ratios, the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via four different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one may operate at the same time.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made.

In another aspect, the agent that promotes or stimulates C3aR and/or C5aR signaling of the cells can be administered directly to or about the periphery of the ischemic tissue by introducing an agent into target cells that causes, increases, and/or upregulates expression of at least one of C3, C5, C3a, C5a, a C3aR agonist, or C5aR agonist in or about the periphery of the ischemic tissue. The at least one of at least one of C3, C5, C3a, C5a, a C3aR agonist, or C5aR agonist is expressed in or about the periphery of the ischemic tissue can be an expression product of a genetically modified cell. The target cells can include cells within or about the periphery of the ischemic tissue or ex vivo cells that are biocompatible with the ischemic tissue being treated. The biocompatible cells can also include autologous cells that are harvested from the subject being treated and/or biocompatible allogeneic or syngeneic cells, such as autologous, allogeneic, or syngeneic stem cells (e.g., mesenchymal stem cells), progenitor cells (e.g., multipotent adult progenitor cells) and/or other cells that are further differentiated and are biocompatible with the ischemic tissue being treated.

In other embodiments, the agent that promotes or stimulates C3aR and/or C5aR signaling of the cells can be administered in combination with a growth factor that promotes angiongenesis or vasculogenesis of the ischemic tissue, mitigates apoptosis of cells of the ischemic tissue, and/or promotes repair of the ischemic tissue. The growth factor can include, for example, VEGF, NGF, GM-SCF, EGF, FGF, IGF, BDNF, BMP, SDF-1, and/or HGF.

In other embodiments, an agent that promotes or stimulates C3aR and/or C5aR signaling can be used to treat wounds in a mammalian subject. The wounds treated by the method and/or compositions can include any injury to any portion of the body of a subject (e.g., internal wound or external wound) including: acute conditions or wounds, such as thermal burns, chemical burns, radiation burns, burns caused by excess exposure to ultraviolet radiation (e.g., sunburn); damage to bodily tissues, such as the perineum as a result of labor and childbirth; injuries sustained during medical procedures, such as episiotomies; trauma-induced injuries, such as cuts, incisions, excoriations, injuries sustained as result of accidents, ulcers, such as pressure ulcers, diabetic ulcers, plaster ulcers, and decubitus ulcer, post-surgical injuries. The wound can also include chronic conditions or wounds, such as pressure sores, bedsores, conditions related to diabetes and poor circulation, and all types of acne. In addition, the wound can include dermatitis, such as impetigo, intertrigo, folliculitis and eczema, wounds following dental surgery; periodontal disease; tumor associated wounds.

It will be appreciated that the application is not limited to the preceding wounds or injuries and that other wounds or tissue injuries whether acute and/or chronic can be treated by the compositions and methods described herein.

The agent that promotes or stimulates C3aR and/or C5aR signaling can also be provided in or on a surface of a medical device used to treat an internal and/or external wound. The medical device can comprise any instrument, implement, machine, contrivance, implant, or other similar or related article, including a component or part, or accessory, which is, for example, recognized in the official U.S. National Formulary, the U.S. Pharmacopoeia, or any supplement thereof; is intended for use in the diagnosis of disease or other conditions, or in the cure, mitigation, treatment, or prevention of disease, in humans or in other animals; or, is intended to affect the structure or any function of the body of humans or other animals, and which does not achieve any of its primary intended purposes through chemical action within or on the body of man or other animals, and which is not dependent upon being metabolized for the achievement of any of its primary intended purposes.

The medical device can include, for example, endovascular medical devices, such as intracoronary medical devices. The medical device may additionally include either implantable pacemakers or defibrillators, vascular grafts, sphincter devices, urethral devices, bladder devices, renal devices, gastroenteral and anastomotic devices, vertebral disks, hemostatic barriers, clamps, surgical staples/sutures/screws/plates/wires/clips, glucose sensors, blood oxygenator tubing, blood oxygenator membranes, blood bags, birth control/IUDs and associated pregnancy control devices, cartilage repair devices, orthopedic fracture repairs, tissue scaffolds, CSF shunts, dental fracture repair devices, intravitreal drug delivery devices, nerve regeneration conduits, electrostimulation leads, spinal/orthopedic repair devices, wound dressings, embolic protection filters, abdominal aortic aneurysm grafts and devices, neuroaneurysm treatment coils, hemodialysis devices, uterine bleeding patches, anastomotic closures, aneurysm exclusion devices, neuropatches, vena cava filters, urinary dilators, endoscopic surgical and wound drainings, surgical tissue extractors, transition sheaths and dialators, coronary and peripheral guidewires, circulatory support systems, tympanostomy vent tubes, cerebro-spinal fluid shunts, defibrillator leads, percutaneous closure devices, drainage tubes, bronchial tubes, vascular coils, vascular protection devices, vascular intervention devices including vascular filters and distal support devices and emboli filter/entrapment aids, AV access grafts, surgical tampons, cardiac valves, and tissue engineered constructs, such as bone grafts and skin grafts.

In other embodiments, the agent that promotes or stimulates C3aR and/or C5aR signaling of the cells can be administered in combination with a growth factor that promotes wound repair and/or mitigates apoptosis of cells of the wound. The growth factor can include, for example, VEGF, NGF, GM-SCF, EGF, FGF, IGF, BDNF, BMP, SDF-1, and/or HGF.

The following examples are for the purpose of illustration only and are not intended to limit the scope of the claims, which are appended hereto.

### EXAMPLE 1

Complement has been shown to be an important component in various pathological responses involving ECs, in all cases the effects have been attributed to serum complement. We tested whether autocrine GPCR signal transduction might be connected with the anti-apoptotic and/or mitotic effects of VEGF on ECs.

### Materials and Methods

### Reagents and Antibodies

VEGF-A was from Prospect (Ness Ziona, Israel). C3aR antagonist (C3aR-A) and C5aR antagonist (C5aR-A) are from Merck (Darmstadt, Germany). Anti-C3 and Anti-C5 was purchased from BD PharMingen (San Diego, Calif.). Anti-C3aR and Anti-C5aR were purchased from Santa Cruz Biotech (Santa Cruz, Calif.). Endothelial cell growth supplement was purchased from BD Biosciences (San Diego, Calif.). Anti-phospho-VEGFR2 (pYpY1054/1059) was purchased from Invitrogen (Camarillo, Calif.).

### Cells & the Isolation of Primary Aortic ECs

bEnd.3 cells and MS1 murine EC lines were cultured in 10% and 5% FBS, respectively with DMEM. The primary mouse aorta endothelial cells (MAECs) were isolated from wild-type C57BL/6J mice (WT), Daf1^{-/-}, C3aR^{-/-}C5aR^{-/-} at ages from 1 to 4 months, by utilizing a non-mechanical and non-enzymatic method. The outgrowth of endothelial cells from aortic rings was markedly facilitated within first 72 h in the absence of antibiotics. Aortic rings thus were discarded at culture day 3 to avoid the possible contamination of non-endothelial cell types. After removing the aortic rings, cells were maintained in completed DMEM/F12 medium consisting of 20% FBS, 2 mM L-glutamine, 1 nonessential amino acid, 0.05 mg/ml endothelial cell growth supplement (ECGS), 100 units/ml penicillin, 100 g/ml streptomycin, and 0.1 mg/ml heparin until confluent.

### Gene Expression and Flow Cytometry

RNA was isolated by the TRIzol method (Invitrogen). Reverse transcription was achieved with Superscript-III reverse transcriptase (Invitrogen) using supplied oligo dT primers. qPCR was performed in a 24 μl volume with SYBR Green PCR mix (Applied Biosystems) using gene specific qPCR primers.

For C3aR and C5aR staining cells were harvested after plating in 10% FBS supplemented DMEM via Versene (Invitrogen) and stained by using three-layer immunoenzyme method. Stained cells were analyzed on a Becton Dickinson LSRII.

### Quantitation of Cell Growth

For studies with bEnd.3 and MS-1 cells, 5×10⁴ cells were plated in 24-well plates and allowed to adhere for 24 hr. Following culturing in 0.5% FBS with DMEM for another 24 hours, the cells were treated as described in the Fig legends. Growth was quantified manually at 24, 48 and 72 hr with trypan blue. At least 95% of cells were viable in all experiments. VEGF-A was used at a concentration of 30 ng/ml, C3a and C5a at 1 ug/ml, and anti-C3 and ant-C3 mAbs at I ug/ml.

### C3a/C5a/VEGF ELISAs and Propidium Iodide Staining

Enzyme-linked immune-adsorbent assays were conducted to quantify C3a and C5a levels in culture supernatants. Ninety six well plates were used and the manufacturer's (eBioscience) protocol followed. FACS without sodium azide was used for diluents with blocking buffer and color was developed using enhanced TMB solution with H₂O₂. The stop solution consisted of 2 N H₂SO₄.

Propidium iodide staining was performed as described previously (ref). In brief, the cells were serum starved for 24 hrs followed by administration of 1 μM colchicine (Sigma). 16 hrs following colchicine treatment, cells were given either growth factor treatment or 17 ng/mL mC5a. After an additional 24 hrs, cells were removed from plating via Trypsin/EDTA and fixed in 0.25% Formaldehyde for 10 mins at 37° C. Cells were spun out of formaldehyde solution and resuspended in 90% methanol at 4° C. until assayed. Following removal from methanol, excess RNA was removed via treatment with RNase (Sigma) and stained with propidium iodide for 30 mins at 4° C. Cells were analyzed on an Epics XL.

### Hypoxia and 2-D HUVEC Tube Formation

The mitochondrial uncoupler protonophore carbonyl cyanide p-(trifluoromethoxy)phenylhydrazone (FCCP)+iodoacetate (IAA), which simulate hypoxia when added to cells, was incubated for 1 hr and 2 hr with HUVEC, and C3, fB, and fD mRNA levels were assayed by qPCR.

Fifty μL of the growth factor reduced matrigel (BD Biosciences) was allowed to polymerize in 96-well plate at 37° C. for 30 minutes. Triplicates of 25,000 HUVEC were plated onto the prepared matrigel in a volume of 150 μL of EBM-2 media (Lonza) in four different conditions: no growth factors, 30 ng/ml of VEGF, 30 ng/ml of VEGF and with 15 ng/ml each of C3aR-A and C5aR-A and the complete set of factors. After approximately 15 hr, images were captured using a light microscope in high magnification.

### Corneal Neovascularization and Tumor Angiogenesis Model

Corneal neovascularization was induced in 6-8 wk-old (C57BL/6) WT, Daf1^{-/-}, C3aR^{-/-}C5aR^{-/-}, and Daf1^{-/-}C3aR^{-/-}C5aR^{-/-} mice (n=5 each group) by placing a non-penetrating suture (0-11 Nylon, Alcon Inc.) in the center of the cornea under a dissecting microscope. On days 7, 9, 11, and 15, corneal vessels were examined after intravenous administration of 100 μl of 2.5% fluorescein-dextran (Sigma) by fluorescence microscopy. The protocol was approved by the Case Western Reserve University Institutional Animal Care and Use Center (IACUC). Male mice were injected subcutaneously with 1.5×10⁶ RM1 prostate cancer cells. Tumors were collected 10 days after injection and were weighed, photographed, snap-frozen in OCT and processed for immunohistochemical staining with biotin-conjugated rat anti-mouse CD31 antibody (BD Biosciences, San Jose, Calif.). Stained sections were analyzed using fluorescent or bright field imaging microscopy (Leica, Germany) and ImagePro Plus Capture and Analysis software version 6.1 (Media Cybernetics). CD31-positive areas were quantified in 5-10 independent fields per tumor implant.

### Results

ECs Locally Synthesize Complement, C3a/C5a are Generated from this Local Synthesis, and Autocrine Interactions of These Anaphylatoxins with C3aR/C5aR on ECs are Essential to Sustain EC Viability

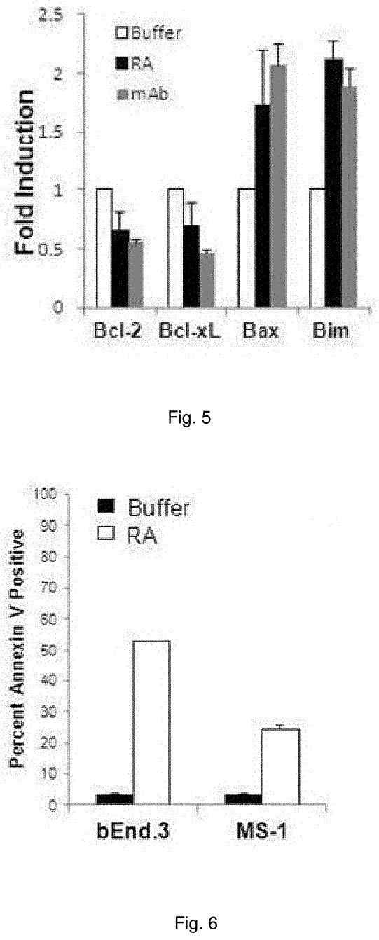

As a first test of whether ECs, like CD4⁺ tonically synthesize complement (under homeostatic conditions) and whether this local synthesis leads to autocrine C3aR/C5aR signaling in ECs, we examined serum free cultures of bEnd.3 and MS-1 murine EC cell lines for local C3a/C5a production and for surface expression of C3aR/C5aR. The culture supernatants of both EC lines contained C3a and C5a and both EC lines expressed both GPCRs (FIG. 4) To test whether tonic C3aR/C5aR signaling provides survival signals to ECs as found for CD4⁺ cells, we added pharmacological C3aR/C5aR antagonists (C3aR-A/C5aR-A) or with anti-C3a/C5a mAbs specific for neo-epitopes in the C3a/C5a ligands (not present on the parental C3/C5 proteins) to the serum free cultures and assayed for markers of apoptosis. Blockade of either the receptors or their ligands evoked surface Fas/FasL expression, caused decreased intracellular Bcl-2/Bcl-x2 and increased Bax/Bim mRNAs, and rendered the ECs susceptible to Annexin V binding (FIGS. 5-6). The effects of the C3aR/C5aR blockade on Bcl-2 mRNA expression in ECs simulated that of blockade of VEGFR2 signaling.

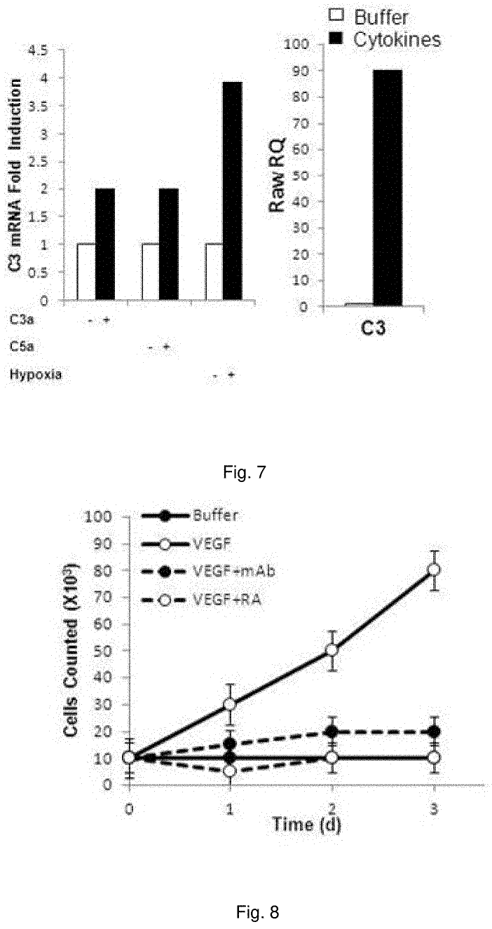

We next conversely assessed how transducing C3aR/C5aR signals into ECs affects endogenous EC complement production and whether it is connected with “classical” EC responses. We incubated HUVEC with 1) inflammatory cytokines, 2) the mitochondrial uncoupler protonophore carbonyl cyanide p-(trifluoromethoxy)phenylhydrazone (FCCP)+iodoacetate (IAA) (which mimicks hypoxia), or 3) C3a or C5a, after which we assayed complement mRNA transcripts by qPCR. The mitochondrial uncoupler evoked an increase in complement transcripts consistent with this signal transduction functioning to amplify EC growth and prevent apoptosis in response to hypoxia (FIG. 7). Added IL-1/IFN-γ/TNF-α had the same effect (FIG. 7). Both added C3a and added C5a increased transcription of C3, indicative of the ability of both anaphylatoxins to establish auto-inductive signaling loops (FIG. 8).

### VEGF Growth Induction Depends on C3aR/C5aR Signaling

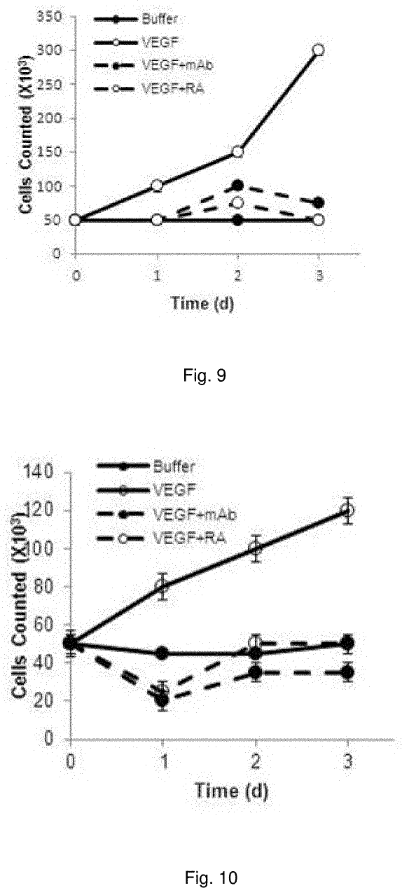

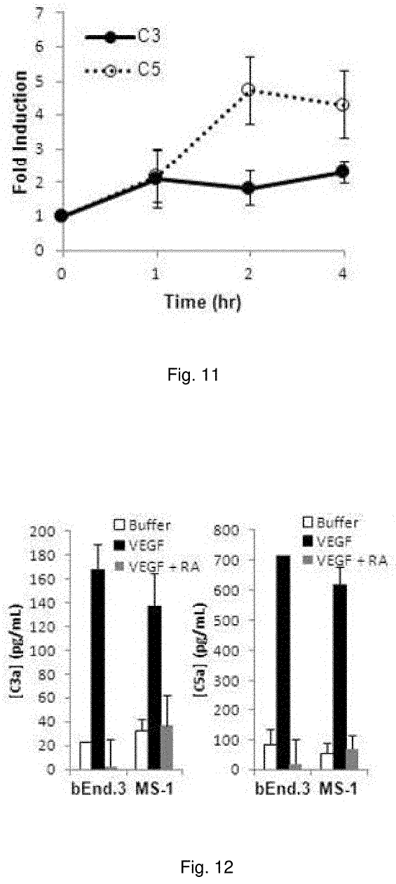

As a first test of whether VEGF growth induction and autocrine C3aR/C5aR signaling in ECs are mechanistically connected, we added VEGF-A to serum starved MS-1 and bEnd.3 cells in the absence or presence of C3aR-A/C5aR-A or anti C3a/C5a mAbs and counted cell numbers over 72 hr. Blockade of either the receptors or their C3a/C5a ligands abolished VEGF-A induced proliferation of both EC lines (FIGS. 8-9). A similar effect was observed in HUVEC cultures (FIG. 10). Blockade of the individual receptors or ligands individually (data not shown) inhibited VEGF-A induced growth and that blockade of both synergized in the inhibition. C3aR/C5aR antagonism or C3a/C5a neutralization did not affect the growth of CTLL cells which is IL-2 dependent and only partially inhibited that of MS-1 or bEnd.3 in 10% plasma verifying that the inhibitory effects on VEGF-A growth induction were specific rather toxic effects on the cells. To determine whether VEGF growth induction is interconnected with upregulated C3aR/C5aR signaling, we examined the effect of added VEGF-A on local complement production by MS-1 and bEnd.3 cells. qPCR of cell extracts and ELISAs of their culture supernatants showed that VEGF-A both up-regulated C3/C5 mRNA transcripts and increased local C3a/C5a production (FIGS. 11-12). These findings show that amplified C3aR/C5aR GPCR signals integrate with VEGF growth signals.

Because the disruptive effect of C3aR/C5aR antagonism on VEGF growth induction could be a consequence of the requirement of autocrine C3aR/C5aR signaling for EC viability, we performed cell cycle assays. Adding C5a to serum starved HUVEC caused transition from G0 into G2 identically to adding VEGF-A. Moreover, C3aR/C5aR blockade prevented VEGF-A triggering of the cell cycle. These findings that autocrine C3aR/C5aR signaling not only prevents EC apoptosis but drives EC growth point to a bidirectional signaling mechanism wherein C3aR/C5aR GPCR signaling transactivates to VEGFR2 signaling and vice versa.

### C3aR/C5aR Signals are Required for VEGF Induced Growth of ECs

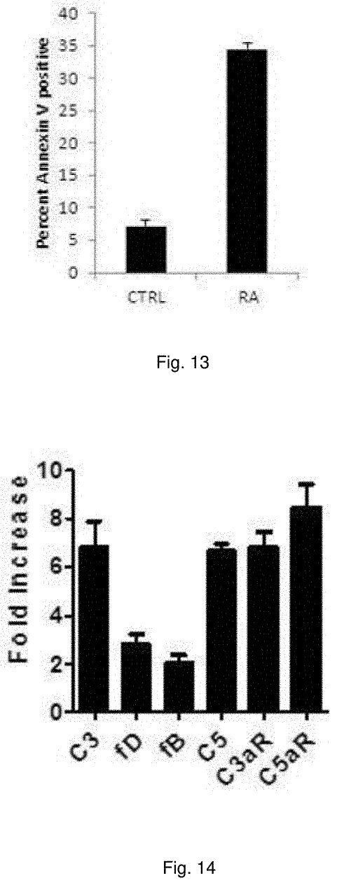

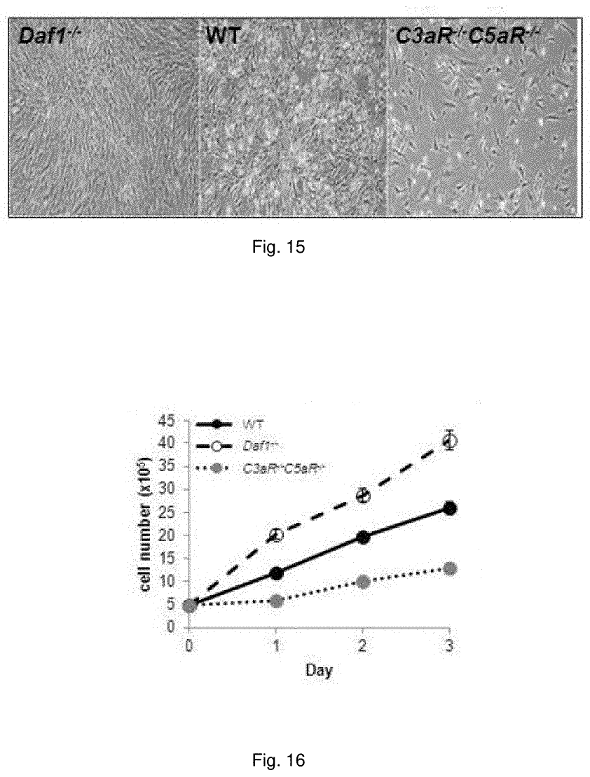

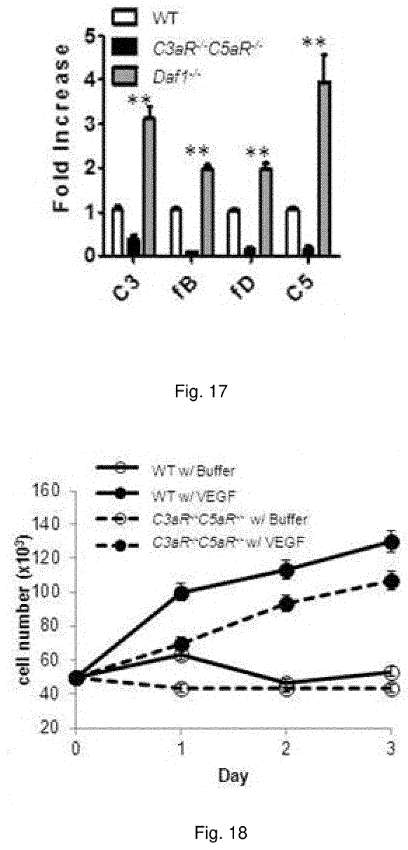

To validate that the above findings apply physiologically, we prepared primary ECs from aortas of WT mice. As found for the bEND.3 and MS-1 EC lines and for HUVEC, aortic ECs from WT mice expressed C3aR and C5aR, antagonizing C3aR/C5aR signaling induced markers of apoptosis (FIG. 13), and VEGF-A upregulated mRNA transcripts of all of the components/receptors associated with autocrine C3aR/C5aR signaling (FIG. 14). We similarly prepared aortic ECs from mice in which C3aR/C5aR signaling is potentiated (Daf1^{-/-} mice) or C3aR and C5aR are deleted (C3aR^{-/-}C5aR^{-/-} mice). Side by side comparisons of the growth rates of the Daf1^{-/-} and C3aR^{-/-}C5aR^{-/-} aortic ECs to that of WT aortic ECs (FIG. 15) showed that the absence of DAF favored EC proliferation (1.77±0.06 vs 1.30±0.017×10⁵ cells 24 hr after plating 10⁵ cells) whereas the absence of C3aR/C5aR virtually abolished it (1.07±0.05×10⁵ cells). Quantitative studies in which we cultured 5×10⁴ ECs from the three genotypes over a 72 hr period and counted cell numbers documented an ˜2-fold faster growth (2×10⁶±0.15 vs 4×10⁶±0.017 p<0.005) of Daf1^{-/-} ECs than of WT ECs (consistent with potentiated C3aR/C5aR signaling), and little or no growth (2×10⁶±0.15 vs 1×10⁶±0.05 p<0.005) of C3aR^{-/-}C5aR^{-/-}ECs (consistent with precluded C3aR/C5aR signaling) (FIG. 16). To establish if the differences in growth rates correlate with differences in EC complement production in vivo, we assayed perfused aortas from Daf1^{-/-}, WT, and C3aR^{-/-}C5aR^{-/-} mice for mRNA transcripts of complement components connected with C3aR/C5aR signaling. qPCR assays documented upregulated and suppressed C3/fB/fD/C5 mRNA transcripts in Daf1^{-/-} and C3aR^{-/-}C5aR^{-/-} aortas, respectively (FIG. 17). To study how the absence of C3aR/C5aR signaling affects VEGF growth induction, we serum starved aortic ECs from WT and C3aR^{-/-}C5aR^{-/-} mice and added VEGF-A to cultures. The genetic absence of C3aR/C5aR markedly suppressed VEGF induced growth (FIG. 18). However, unlike the virtual abrogating effects of disrupted C3aR/C5aR signaling in WT ECs with the antagonists or mAbs, VEGF-A retained some growth inductive capacity, an issue that will addressed below.

### C3aR/C5aR Signaling in ECs is Essential for Vascular Tube Formation and for Angiogenesis In Vivo

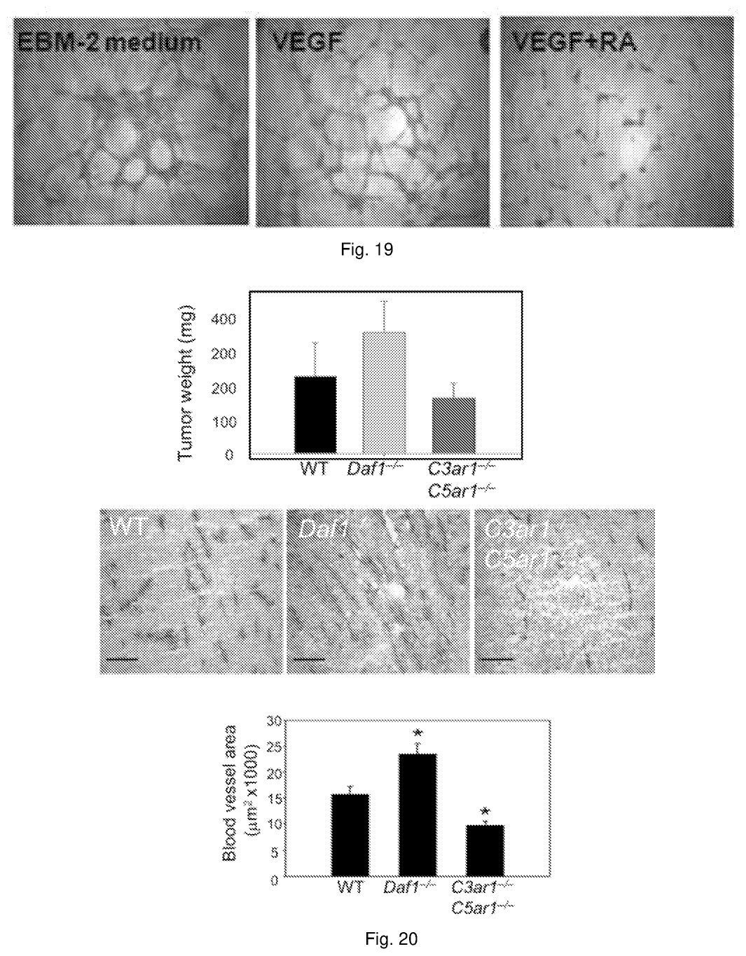

As a first test of whether the above connection of VEGF growth induction with autocrine C3aR/C5aR signaling is physiologically relevant, we performed two-dimensional tube formation assays with HUVEC to determine how this GPCR signaling affects angiogenesis. Culturing HUVEC under conventional conditions with EBM-2 basal medium containing supplemental growth factors yielded the typical growth pattern of an EC tubal network consisting of cluster regions with a few braches. (FIG. 19, Left). Substitution of exogenous VEGF for the growth factor cocktail evoked increased branch points (FIG. 19, Middle). In contrast, the inclusion of C3aR-A/C5aR-A together with VEGF-A under identical conditions essentially disrupted tube formation (FIG. 19, Right).

To establish whether in vivo angiogenesis, in fact, depends on C3aR/C5aR signaling, we used two in vivo models. In the first model, we placed non-penetrating sutures in the corneas of WT, Daf1^{-/-}, C3aR^{-/-}C5aR^{-/-} and Daf1^{-/-}C3aR^{-/-}C5aR^{-/-} mice. This assay has the advantage of measuring neo-angiogenesis (new blood vessel growth) since the cornea is normally avascular. Beginning at day 5, we examined corneas by fluorescence confocal microscopy after intravenously administering fluorescent dextran beads. In contrast to the gradual influx of blood vessels at the corneal margins starting at day 9 in WT mice, markedly accelerated and more robust influx occurred in Daf1^{-/-} mice, and virtually none occurred in C3aR^{-/-}C5aR^{-/-} or Daf1^{-/-}C3aR^{-/-}C5aR^{-/-} mice.

In the second model, we implanted RM1 prostate tumors which are highly vascularized and dependent on angiogenesis for their progression (Huang et al., 2005) into the flanks of male WT, Daf1^{-/-} and C3aR^{-/-}C5aR^{-/-} mice. We harvested the tumors at day 14 post inoculation, weighed them, and immunohistochemically examined sections of their stroma staining for the EC marker CD31. The tumors in C3aR^{-/-}C5aR^{-/-} mice were smaller (227.93±201.28 vs 164.33±89.7, p=0.29) than in WT mice, and larger (227.93±201.28 vs 359±185.31, p=0.18) than in Daf1^{-/-} mice (FIG. 20, Top). The sections showed significantly reduced vessel density and vascular area in tumors grown in C3aR^{-/-}C5aR^{-/-} mice compared to WT mice (9772±799 vs. 15699±1591 μm², P=0.004, n=8) (FIG. 20, Bottom). In contrast, angiogenesis was significantly enhanced in tumors from Daf1^{-/-} mice (23458±1976 vs. 15699±1591 μm², P=0.01, n=8).

### C3aR/C5aR Signaling is Essential for VEGFR2 Phosphorylation

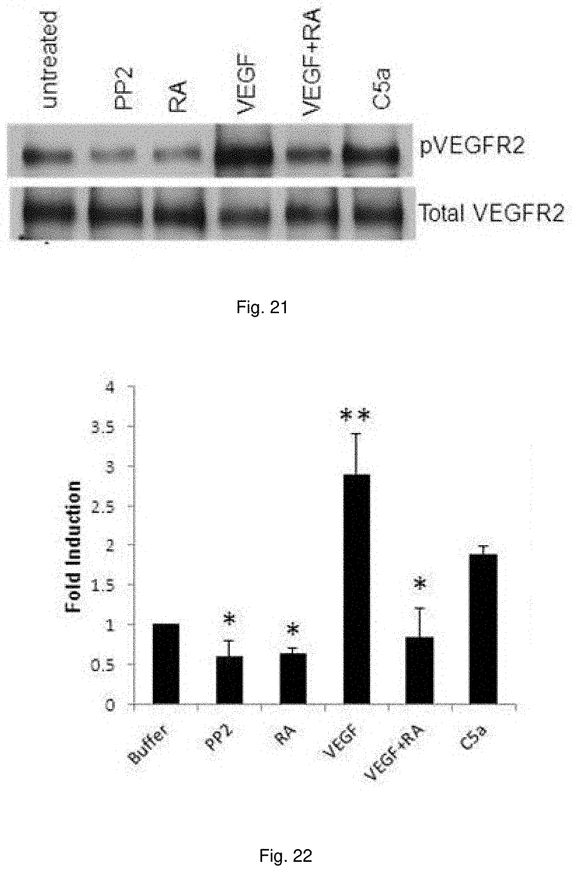

To directly establish whether C3aR/C5aR signaling is integral to VEGF signaling, we next evaluated phosphorylation of VEGFR2. We tested 1) how adding C5a to primary WT ECs affects VEGFR2 autophosphorylation and 2) whether VEGFR2 phosphorylation induced by added VEGF-A is affected by antagonizing C3aR/C5aR signaling. We incubated serum starved WT aortic ECs with C5a or with VEGF-A in the absence or presence of C3aR-A/C5aR-A, after which we probed immunoblots of protein lysates and anti-pVEGFR2^{Y1054/1059} mAb or pan VEGFR2 antibody. The added C5a increased VEGFR2^{Y1054/1059} phosphorylation relative to untreated controls and the C3aR/C5aR blockade virtually abolished VEGF-A induced VEGFR2 auto-phosphorylation (FIG. 21-22).

Up-Regulation of VEGFR2 and Endogenous VEGF-A Production Compensates for Pro-Apoptotic Signaling in C3aR^{-/-}C5aR^{-/-} ECs

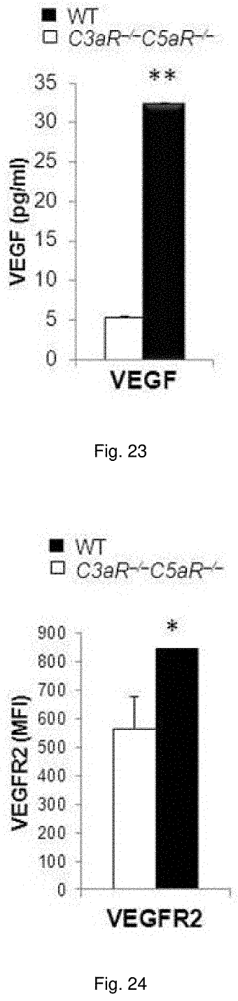

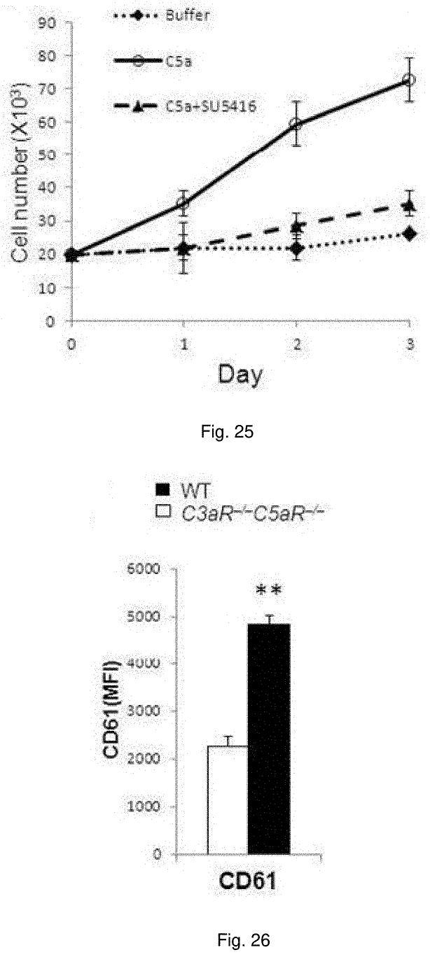

The above experiments with WT ECs in this Example showed that autocrine C3aR/C5aR signaling provides survival signals and that blockade of this signaling triggers both the intrinsic and extrinsic PCD pathways. We compared VEGFR2 expression levels and endogenous VEGF-A production in C3aR^{-/-}C5aR^{-/-} and WT ECs. ELISAs of their supernatants of untreated the C3aR^{-/-}C5aR^{-/-} ECs (FIG. 23) showed 5-fold more VEGF-A production compared to WT ECs, and flow cytometry (FIG. 24) showed 2-fold increased levels of VEGFR2 on their surfaces. Addition of the VEGFR2 inhibitor SU5416 to cultures of C3aR^{-/-}C5aR^{-/-} ECs without or with added C5a (FIG. 25) abolished their growth, indicating that upregulated VEGFR2 auto-phosphorylation compensated for the loss of C3aR/C5aR signaling in the knockouts and that EC viability depended entirely on this compensation. Because β3 Integrin (CD61) has been reported to be regulated by VEGFR2 signaling and implicated in EC survival signaling, we compared CD61 expression in C3aR^{-/-}C5aR^{-/-} and WT ECs. Flow cytometry analysis of C3aR^{-/-}C5aR^{-/-} ECs showed 2.5-fold increased CD61 levels (FIG. 26).

### VEGF Induces C3aR/C5aR Signaling by an IL-6 and Stat3 Dependent Mechanism

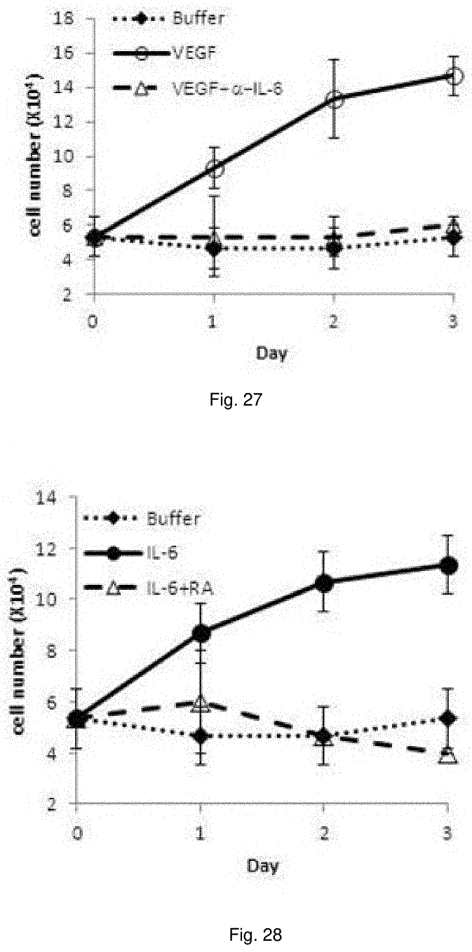

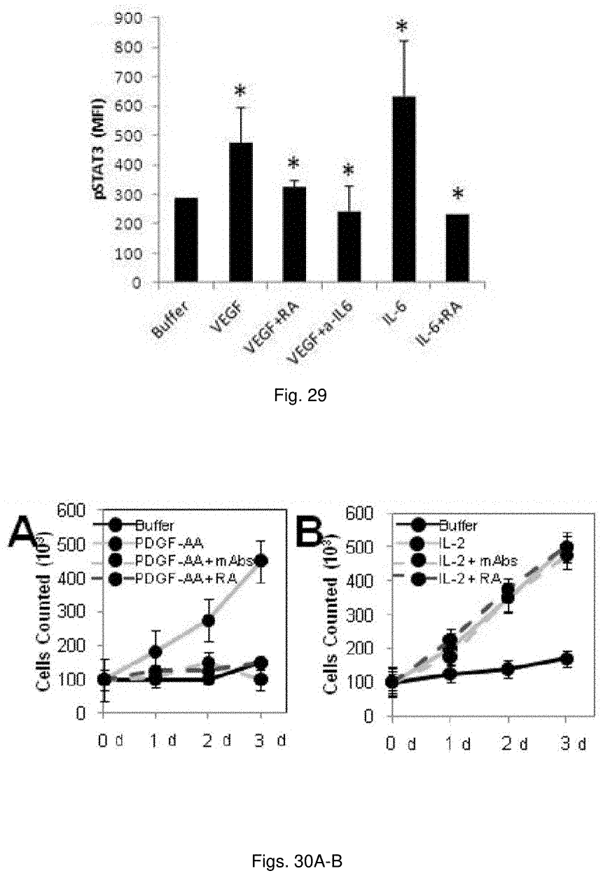

While the experiments above showed that C3aR/C5aR signaling transactivates to phosphorylate VEGFR2, they left unanswered how VEGFR2 signaling is mechanistically linked to C3aR/C5aR signaling. Numerous past studies have linked VEGF with both IL-6 and p-Stat3 our prior studies by ourselves have shown that C5a induces IL-6 and that IL-6 signaling is interconnected with C3aR/C5aR signaling. To determine if the linkage between VEGF and C3aR/C5aR signaling in ECs involves IL-6, we incubated MS-1 ECs with 1) VEGF-A alone, IL-6 alone, or VEGF-A plus anti-IL-6 mAb, or with 2) IL-6 alone or IL-6 plus C3aR-A/C5aR-A, and assayed cell growth. Growth induction by IL-6 induced EC growth comparably to VEGF-A and VEGF-A's growth induction was abolished by anti-IL-6 (FIG. 27). The growth induction of ECs of IL-6, like that of VEGF-A, was abolished by C3aR-A/C5aR-A (FIG. 28). Both VEGF-A and IL-6 induced Stat3 phosphorylation and the Stat3 phosphorylation in both cases was abolished by C3aR/C5aR antagonism (FIG. 29). VEGF-A treatment or WT aortic ECs upregulated C3/C5 and increased local C3a/C5a generation as found in FIGS. 11-12 for MS-1 ECs, but neither change occurred in the presence of anti-IL-6 mAb or the JAK1 inhibitor (Data not shown). These finding thus indicate that VEGF interconnects with C3aR/C5aR signaling via the induction of IL-6 and its activation of Stat3.

The data show that targeting C3aR and C5aR can repress pre cancer or cancer associated angiogenesis. Based on the data in this study mechanistically connecting C3aR/C5aR signaling with VEGFR2 signaling, such targeting should have value in other conditions including diabetic retinopathy rheumatoid arthritis (RA) where it has been shown that hypoxia and consequent angiogenesis augments the proliferation of synovial fibroblasts, systemic sclerosis where it has been shown that VEGF induced EC growth contributes to uncontrolled fibroblast growth and external ear canal cholesteatoma (EACC), an invasive and destructive external otitis, where it has been shown that migration of keritinocytes into the external ear is connected with increased expression of VEGF in all layers of the EACC-epithelium.

### EXAMPLE 2

Inflammatory cell influx and vascular cell proliferation underlie atherosclerotic progression and set the stage for thrombosis which eventuates in myocardial infarction and stroke. While many cell surface molecules and inflammatory mediators have been implicated in this self perpetuating process, the mechanisms that drive inflammation and proliferation remain incompletely characterized. We have found that both smooth muscle cells (SMCs) and monocytes/macrophages (mΦs) locally produce C3a and C5a activation fragments and that these anaphylatoxins interact with upregulated C3a and C5a receptors (C3aR and C5aR) on SMCs/mΦs. Our studies show that amplification of this signal transduction is what drives SMC/mΦ proliferation and evokes mΦs inflammatory cytokine production. This insight derived from our studies of immune cell activation which uncovered the previously unrecognized fact that local complement synthesis by interacting dendritic cells (DCs)- and T cells is an early event in T cell activation and that the resulting C3a/C5a-C3aR/C5aR interactions play a requisite role in T cell proliferation and effector cytokine, e.g., IFN-γ/IL-17 production. These studies in immune cells further showed that C3aR/C5aR signaling operates tonically to maintain T cell viability and suppress costimulatory molecule and innate cytokine, e.g., IL-1β/IL-12/IL-23 production. Importantly, our studies in SMCs/mΦs show that C3aR/C5aR signal transduction functions tonically similarly to that in immune cells. Our work has shown that the generation of C3a/C5a from locally synthesized complement by SMCs/mΦs, like that from immune cells, is regulated by the cell surface C3/C5 convertase inhibitor DAF and our studies now show that DAF expression is controlled by Kruppel-like factor 4 (KLF4). We have found that C3aR/C5aR signaling drives the neointimal response to endothelial cell (EC) injury. Our preliminary studies show that the mechanism underlying this is that amplified C3aR/C5aR signal transduction is essential for growth induction by platelet derived growth factor (PDGF), a factor important in both the EC response to injury and atherogenesis. In this Example, we 1) further characterized the interconnections of C3aR/C5aR signal transduction with platelet, leukocyte, EC, and SMC responses to EC injury, and found 2) the connections of C3aR/C5aR signal transduction with atherogenesis and thrombosis.

We found most cell types locally synthesize complement, and this local complement synthesis controls many cellular responses. Of particular interest, are findings documenting the local generation of C3a/C5a and the interaction of these fragments with C3aR/C5aR on endothelial cells (ECs), smooth muscle cells (SMCs), as well as myeloid cells, all of which are relevant to atherogenesis and possibly to its thrombotic sequelae. Local synthesis and activation in an autocrine fashion sustains EC/SMC viability and alters cellular production of and/or response to growth factors and cytokines. Importantly, this signaling loop is regulated by the cell surface C3/C5 convertase inhibitor, decay accelerating factor (DAF or CD55). Downregulation of DAF potentiates while upregulation suppresses C3aR/C5aR signaling and attendant effects on cellular viability and activation.

We found that vascular inflammation and neointimal formation are potentiated in mice deficient in Daf1 (the murine homolog of the human DAF gene), but completely attenuated in Daf1^{-/-}C3aR^{-/-} and Daf1^{-/-}C5aR^{-/-} mice. Data indicate that hypoxia and pro-inflammatory cytokines upregulate local EC complement production and local C3a/C5a generation, promoting leukocyte recruitment and activation. Finally, we have found that DAF expression is regulated by Kruppel like factor 4 (KLF4) in both ECs and immune cells. Based on these newly uncovered interconnections of autocrine C3aR/C5aR signaling in ECs, SMCs, and leukocytes, we show that local complement synthesis and autocrine C3aR/C5aR signaling regulates vascular cell responses to injury, which in turn contributes the development of atherosclerosis and thrombosis.

We found that autocrine/paracrine C3aR/C5aR signaling in ECs/monocytes/mΦs/SMCs is mechanistically linked to key proliferative and inflammatory processes that underlie the neointimal response of ECs to injury and participate in atherosclerotic progression to thrombosis. Evidence below implicates local C3aR/C5aR signaling in vascular proliferation, inflammation, injury, and repair.

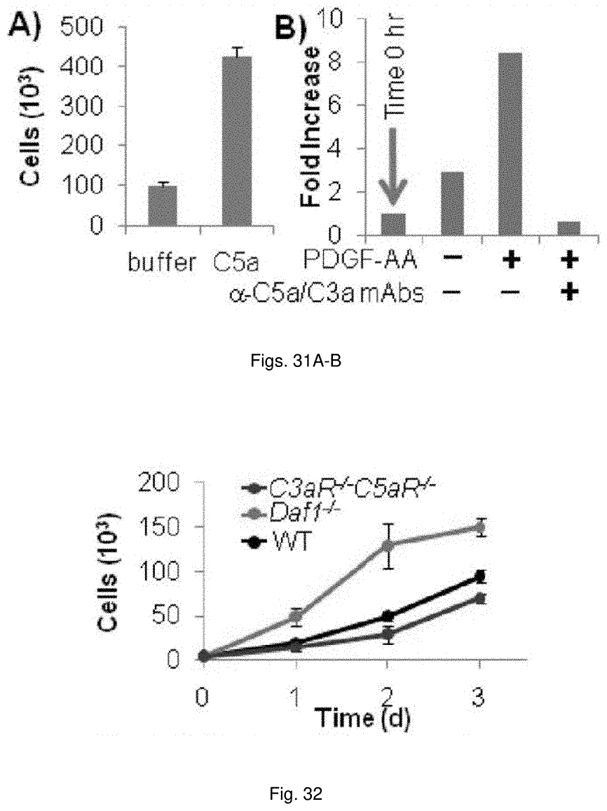

PDGF contributes to atherogenesis and plays a central role in neointimal proliferation following EC injury. In view of the growth difference of primary cultured Daf1^{-/-}, WT, C3aR^{-/-}C5aR^{-/-}, and C3^{-/-}C5^{-/-}SMCs, we examined whether C3aR/C5aR signals are interconnected with PDGF growth induction. For these studies, we utilized NIH-3T3 fibroblasts (which express PDGFR-aa) in conjunction with PDGF-AA. Following determination of optimal PDGF-AA doses, we incubated 1×10⁵ cells/well in triplicate with 30 ng/ml of PDGF-AA±10 ng/ml of C3aR and C5aR antagonists (C3aR-A/C5aR-A), 10 ng/ml of anti-C3a/anti-C5a mAbs or respective controls, after which we quantified cell growth by counting cell numbers over time. Remarkably, blockade of either the receptors or their C3a/C5a ligands near completely suppressed PDGF-AA induced proliferation (FIG. 30A). Consistent with specificity, the growth of CTLL cells which is IL-2 dependent was not affected by C3aR/C5aR antagonism (FIG. 30B) and that of NIH-3T3 cells in 10% plasma was only partially inhibited. To directly establish whether C5aR signals augment PDGF-AA growth signaling, we 1) quantified the effect of added C5a one growth, and 2) assayed C5a in culture supernatants of PDGF-AA treated cells. This showed that a) added C5a (FIG. 31A) by itself induced proliferation, b) NIH-3T3 cells tonically produce C5a, and c) C5a generation by NIH-3T3 cells is amplified by PDGF-AA (FIG. 31B). Because the dependence of PDGF growth induction on C3aR/C5aR signaling could be indirect, i.e., a consequence of its requirement for viability, we performed cell cycle assays. Adding C5a to serum starved NIH-3T3 cells caused transition from G0 into G2 identically to adding PDGF indicating that autocrine C3aR/C5aR signals not only prevent apoptosis but drive growth.

To validate that these findings apply physiologically, we repeated the PDGF studies with the primary cultures of SMCs from aortas of WT, Daf1^{-/-}, and C3aR^{-/-}C5aR^{-/-} mice. Daf1^{-/-} SMCs (in which C3aR/C5aR signaling is potentiated) showed greater proliferation than WT SMCs in response to PDGF-AA, and C3aR^{-/-}C5aR^{-/-} SMCs showed reduced proliferation. These findings show that C3aR/C5aR signal transduction in SMCs is important to their proliferative response in EC injury and atherogenesis. Results below show that a) atherogenesis is accelerated in the absence of DAF and b) the neointimal response to wire injury is markedly heightened in Daf1^{-/-} mice but suppressed below that in WTs in Daf1^{-/-}C3aR^{-/-} and Daf1^{-/-}C5aR^{-/-} mice are consistent with this interpretation.

Antagonizing C3aR/C5aR signal transduction induces the synthesis and activation of TGF-β1. We investigated whether TGF-β1 production might underlie the growth suppression connected with C3aR/C5aR blockade. To test this, 1) we assayed supernatants from NIH-3T3 cells treated with PDGF-AA+anti-C3a/anti-C5a mAbs and 2) following removal of anti-C3a/anti-C5a from the mAb treated NIH-3T3 cell cultures (with protein-G beads), we added supernatants without or with anti-TGF-β1 blocking antibody to fresh cultures of PDGF-AA treated NIH-3T3 cells. These analyses showed that antagonizing C3aR/C5aR signaling induced active TGF-β1 expression (+2.5 ng/ml) and that the elicited TGF-β1 suppressed PDGF-AA induced growth (5-fold). RT-PCR of PDGF-AA treated NIH-3T3 cells showed that PDGF-AA suppressed TGF-β1 mRNA transcription (˜3-fold), whereas C3aR/C5aR antagonism induced it. Activation of latent TGF-β1 can be dependent on Thrombospondin 1 (Tsp-1). Addition of anti-Tsp-1 Ab blocked the generation of active TGF-β1, indicating that Tsp-1 induction is also dependent on C3aR/C5aR antagonism (a result relevant to the pro-atherogenic phenotype of Tsp-1^{-/-} mice). Collectively, these findings indicate that 1) disruption of C3aR/C5aR signaling evokes TGF-β1 production, 2) TGF-β1 enters into an autocrine signaling loop, and 3) C3aR/C5aR signals and TGF-β1 signals oppose each other in regulating PDGF induced growth.

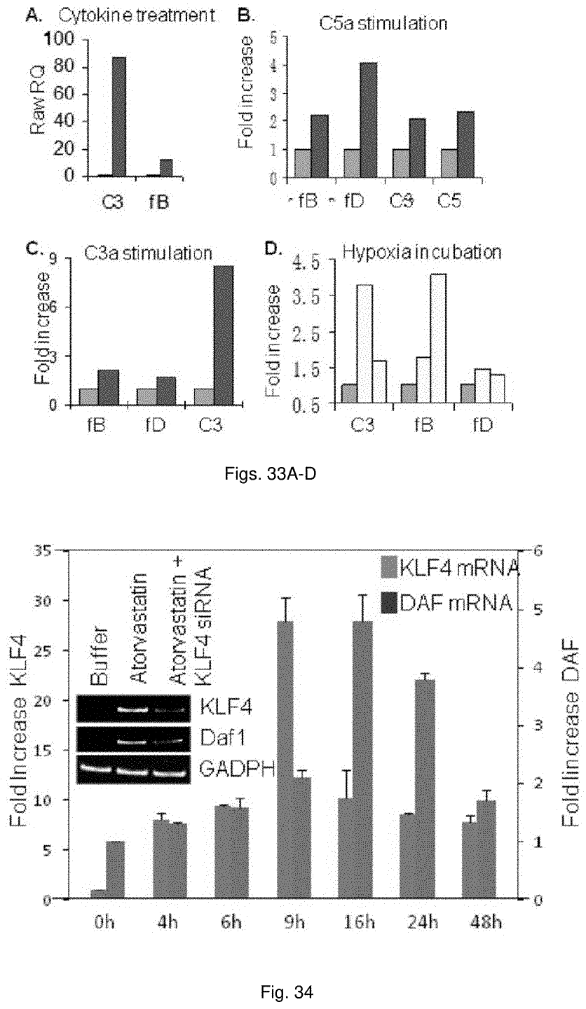

ECs and leukocytes locally synthesize C3/fB/fD/C5 and the elicited C3a/C5a signal through C3aR/C5aR on the same cells to amplify this synthesis. To determine whether ECs locally synthesize AP complement components, we added IL-1/IFN-γ/TNF-α to HUVEC, 1 hr after which we quantified C3 and fB transcripts by qPCR. This analysis (FIG. 33A) showed marked upregulation of both mRNAs. Consistent with the ability of C3a/C5a to feed back through C3aR/C5aR and initiate an auto-inductive signaling loop, added C3a or C5a caused the ECs to make more AP components (FIGS. 33B-C). Incubation of HUVEC with the mitochondrial uncoupler protonophore carbonyl cyanide p-(trifluoromethoxy)phenylhydrazone (FCCP)+iodoacetate (IAA) (which simulate hypoxia when added to cells) also increased HUVEC complement synthesis (FIG. 33D). To test whether leukocytes that are recruited to sites of EC injury locally synthesize AP components and C5a similarly feeds back in them to amplify local complement production, we stimulated Raji, Jurkat, U937 cells, primary mΦs, B cells, and DCs with 10 nM C5a. This documented induction of AP and C5 gene transcription by qPCR.

### DAF Attenuates Atherogenesis

To determine whether DAF's inhibition of C3/C5 activation retards atherogenesis, we crossed Daf1^{-/-} mice with ApoE^{-/-} or ldlr^{-/-} mice, the two strains widely used to assess experimental atherosclerosis following high fat feeding. After placing homozygous Daf1^{-/-}ApoE^{-/-} and Daf1^{-/-}ldlr^{-/-} and their respective Daf1^{+/+} littermates on a high fat diet to accelerate atherogenesis, we initially compared their coronary arteries and aortas for plaques at wk 11. On both backgrounds, immunohistochemical analyses showed 1.5-2-fold larger plaques (μ2/section), Moreover, there was more C3b deposition in DAF's absence. In a second cohort, we quantified lipid accumulation by Oil Red-O staining after 17 weeks of a high fat diet. Both gross and histologic analyses (FIG. 34) revealed a robust increase in atherosclerotic burden in Daf1-null animals.

In view of the connections of MCP-1 and other proinflammatory cytokines with atherosclerosis and the mechanistic linkage of DAF deficiency with potentiated C3aR/C5aR transduction, we tested whether C5aR signaling induces proinflammatory gene transcription. Added C5a induced the mRNA transcription of chemotactic MCP-1/IL-8, proinflammatory IL-1β/IL-6 and growth inductive GM-CSF.

### DAF Regulates the Biological Response to EC Injury

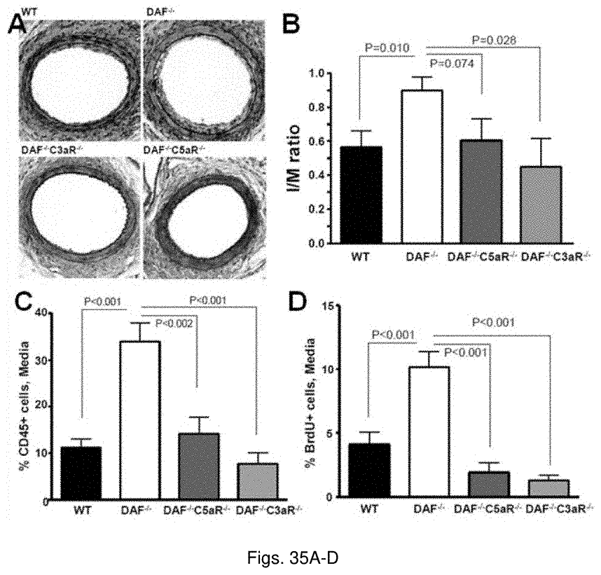

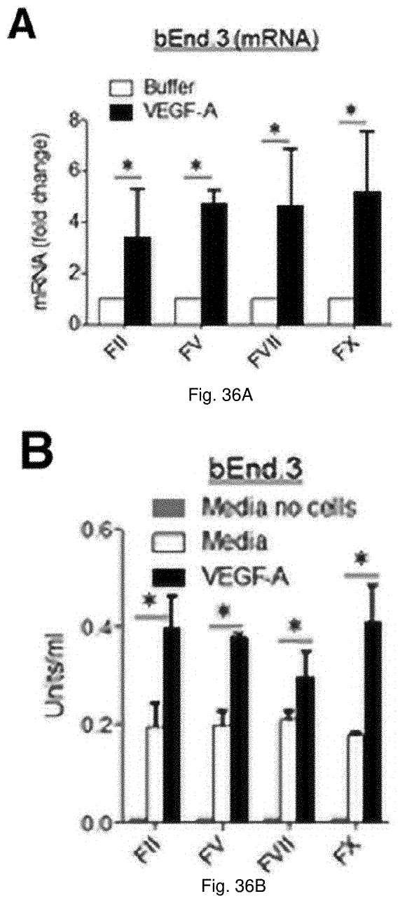

To directly test whether DAF regulates inflammatory and proliferative responses to EC injury, we compared femoral artery wire injury in Daf1^{-/-} mice and WT controls. In this model, wire injury is accompanied by EC denudation, platelet and fibrin deposition, and prominent vascular inflammation. This model is used to closely mimic restenosis as seen following angioplasty in human patients. In WTs, we found that intimal thickening was evident 5 days post injury and further progressed between 5 and 14 days. Remarkably in Daf1^{-/-} mice, at day 14, intimal thickening was 155% greater (p=0.026) (FIG. 35A) and the intima:media area ratio (I:M) was ˜2-fold increased (0.90±0.20 vs. 0.57±0.41; p=0.01) compared to WT mice. Immunohistochemical staining for C3dg, a C3b fragment, was significantly increased in Daf1^{-/-} vs WT vessels (51.1±8.3% vs 30.6±10.0%; p=0.008; data not shown; FIG. 35B). In addition, Daf1^{-/-} vessels exhibited increased immune cell infiltration (CD45+ staining; FIG. 35C) and cellular proliferation (BrdU staining; FIG. 35D). These studies identify a direct role for DAF in the proliferative and inflammatory response to mechanical vascular injury. To establish whether the mechanism by which its deficiency alters the vascular response to EC injury is increased by C3aR and/or C5aR signaling, we repeated the studies in Daf1^{-/-}C5aR^{-/-} and Daf1^{-/-}C3aR^{-/-} mice. We found that the augmented vascular inflammation, cellular proliferation, and neointimal thickening observed in Daf1^{-/-} mice decreased to, or even below, WT levels in each KO animal (FIG. 35). In summary, the results demonstrated that DAF control of C3a/C5a generation and consequent C3aR/C5aR signaling is critical for the biological response to EC injury. These finding were recently confirmed in studies employing C3aR/C5aR antagonists.

### EXAMPLE 3

In this example, C5a was recently linked with physiological VEGF-A growth induction. However, based on prior immune cell activation studies, it was theorized to be generated by the conventional alternative pathway C5 convertase C3bBbC3b. These studies demonstrated that interacting dendritic celleCD4⁺ cell partners each generate C5a (and C3a) from complement components that are endogenously synthesized by both cell types, and that the GPCR signaling plays an integral role in shaping the T-cell response. Analogous to the findings in immune cells, the studies of C5a production by ECs showed that autocrine C5ar1 signaling plays a requisite role in vascular endothelial cell growth factor receptor 2 (VEGFR2) mitotic signaling. It promotes VEGFR2 autophosphorylation, downstream growth signaling via the canonical VEGFR2 cascades, and EC transition through the cell cycle.

The findings of local complement production in ECs together with the observation that thrombin can cleave C5a from C5 in vitro prompted the hypothesis that ECs might endogenously produce coagulation factors in addition to complement components, and that EC produced thrombin might participate in EC C5a generation. Therefore, this study investigated whether endogenous thrombin generation, if locally produced within ECs, might be mechanistically linked with endogenous C5a generation in ECs, and whether these linkages might participate in EC proliferation and angiogenesis.

### Materials and Methods

### Cells, Antibodies, and Reagents

The sources of cells and major reagents used in each assay are given with each assay method. All cells and reagents used are listed together in detail in the major resource table (Table 1).

### Growth Assays and Real-Time Quantitative PCR

Murine bEnd 3 and NIH-3T3 cell lines were maintained in Dulbecco's modified Eagle's medium containing 10% fetal bovine serum. Human umbilical vein endothelial cells (HUVECs) and human aortic endothelial cells (HAECs) were maintained in EBM-2 media containing BulletKit (Lonza, Basel, Switzerland) without added vitamin K. Except where indicated, cells were seeded at 2.5×10⁴ per well in 24-well plates in complete media, after which they were transferred to media containing 0.5% serum and treated as described in the figure legends. The concentrations of C5a, C5ar1-A, thrombin, and PAR1 and PAR4 agonists and antagonists were chosen on the basis of previous studies and the manufacturer's directions. Cell numbers were counted manually. Real-time quantitative PCR was performed as described. Data are given as fold increases relative to basal levels corrected for actin or glyceraldehyde-3-phosphate dehydrogenase.

### Coagulation Assays

A total of 100 mL of culture supernatant was incubated at 37° C. for 5 minutes with 100 mL of plasma deficient in FII, factor V (FV), factor VII (FVII), or factor X (FX; George King Biomedical, Overland Park, Kans.). A total of 100 mL of Phospoplastin RL (R2 Diagnostics, South Bend, Ind.) was then added, and time to coagulation was measured while continuously mixing. Percentage clotting activity corresponds to the dilution of pooled human plasma (George King Biomedical), yielding the same clotting time.

### Enzyme-Linked Immunosorbent Assays and Flow Cytometry

Enzyme-linked immunosorbent assays for C5a and flow cytometry for phosphorylated STAT3 were performed as described. Intracellular staining was performed following permeabilization, as in past studies, and the intracellular signal was calculated following subtraction of the extracellular signal.

### GSK-3b Assay

The assay was performed using the glycogen synthase kinase (GSK)-3b Activity Assay kit (Sigma-Aldrich, St. Louis, Mo.; catalog number CS0990), per the manufacturer's protocol.

### Sprouting Assay

Thoracic aortae were obtained from 8- to 14-weekeold mice following perfusion with Ringer's solution. Following removal of fibroadipose tissue, approximately 1-mm segments were suspended in a 6-mm petri dish containing complete EGM-2 (Lonza) with reduced growth factor Matrigel (Corning, Tewskbury, Mass.). Aortic ring sprouts were imaged on days 6 to 8, and sprout areas were determined by morphometric analysis using MetaMorph (Molecular Devices, San Jose, Calif.) and a Leica DMI6000 microscope (Wetzlar, Germany).

### Matrigel Plug Angiogenesis Assay

Reduced growth factor Matrigel (as used in sprouting assays; 500 mL) supplemented with heparin (60 U/mL) and VEGF-A (300 ng/mL) was injected subcutaneously into the lower abdominal flank of anesthetized 8- to 14-weekeold wild-type (WT) C57BL/6 mice. Ten days after injection, recovered Matrigel plugs were fixed in formalin, embedded in OCT compound, and sectioned. Sections were stained for different coagulation factors [anti-FII (Aviva Systems Biology Corp., San Diego, Calif.; ARP41757-P050; lot number QC12431), anti-FV (Bioss, Woburn, Mass.; bs-1040R; lot number AD121701), anti-FVII (Bioss; bs-4846R; lot number YE1022W), or anti-FX (Bioss; bs-9501R; lot number 9B26M7)] followed by AF546-labeled anti-rabbit. After coagulation protein staining, cells were counterstained with AF488-labeled anti-CD31 (clone 390; BD Biosciences, East Rutherford, N.J.), mounted in ProLong Gold anitfade reagent with DAPI (Molecular Probes, Eugene, Oreg.; catalog number P36935), and visualized by confocal microscopy. Images were collected using a 63× (1.4 numerical aperture) oil immersion objective on a Leica HyVolution SP8 inverted confocal microscope. Resulting images were subjected to deconvolution using Hyugens Professional (Hilversum, The Netherlands).

### Confocal Microscopy

For C5ar1 and PAR4 colocalization assays, bEnd.3 cells were grown overnight on ibiTreat 8 chamber m-Slides (ibidi, Martinsried, Germany). The slides were washed three times with 1× phosphate-buffered saline and fixed with 2% formaldehyde. Cells were stained with a cocktail containing 2 mg/mL of each of antieC5ar1-AF488 (Abd Serotec, Oxford, UK) and antiePar4-AF546 (Abcam, Cambridge, UK) for 1 hour. For Matrigel plug assays, plugs were implanted into the inner thighs of WT mice for 10 days. Plugs were harvested, embedded in OCT compound, and sectioned. Sections were stained with anti-FII, anti-FV, anti-FVII, or anti-FX, followed by AF546-labeled anti-rabbit secondary antibody (Ab). Cells were counterstained with anti-CD31 (green). Nuclei were visualized with DAPI (blue). For coagulation factor staining in confluent versus nonconfluent cells, HUVECs were grown to 70% and 100% confluence, fixed with 2% formaldehyde, and blocked with 5% horse serum in phosphate-buffered saline. Cells were stained with anti-FII followed by AF546-labeled anti-rabbit secondary Ab.

All images were collected using a 63× (1.4 numerical aperture) oil immersion objective on a Leica HyVolution SP8 inverted confocal microscope. Resulting images were subjected to deconvolution using Hyugens Professional.

### Co-Immunoprecipitation Assays

Cells were lysed in 1× Cell Lysis Buffer (10×; Cell Signaling, Danvers, Mass.); catalog number 9803) supplemented with 1 mmol/L phenylmethylsulfonyl fluoride and 1 Complete Mini protease inhibitor tablet (Roche, Boston, Mass.; catalog number 11836153001) for 10 minutes on ice (1 mmol/L Na orthovanadate was used to inhibit phosphatase activity when lysates were to be assayed for kinase activity). Lysates were then sonicated three times for 2 minutes to break apart nucleic acids, after which the cells were centrifuged for 10 minutes at 12,000×g. Clean supernatants were transferred to new tubes and incubated with Protein-A/G beads (Santa Cruz Biotechnology, Dallas, Tex.) to preclear the lysates and prevent nonspecific co-immunoprecipitation. Abs against PAR4, C5arl, GSK-b, and CD59 were added, and samples were incubated overnight at 4° C. Protein-A/G beads were used to pull down Ab, and immunoprecipitations were assayed by Western blot analysis, enzyme-linked immunosorbent assay, or protein activity via kits, as described.

### BRET Data

Bioluminescence resonance energy transfer (BRET) analyses were performed as described. Briefly, HEK293 cells (1×10⁵) were transfected with 0.5 mg of Par4-Luc donor plasmid and increasing amounts (0 to 5 mg) of C5ar1egreen fluorescent protein (GFP) acceptor plasmid or C5ar1-Luc donor plasmid and increasing amounts (0 to 5 mg) of C5ar1-GFP or Par4-GFP acceptor plasmids. Emission was detected using a PerkinElmer Life Sciences (Waltham, Mass.) Victor 3 plate reader equipped with the appropriate BRET2 filter set (410 nm with 80-nm bandpass and 515 nm with 30-nm bandpass; PerkinElmer Life Sciences). Emission at 410 and 515 nm was collected immediately after the addition of 5 mmol/L luciferase substrate (coelenterazine 400a; Biotium Inc., Hayward, Calif.). BRET signal was calculated by the ratio of emission at 515 nm to emission at 410 nm minus the BRET in the absence of GFP. In BRET studies, specific interactions are detected by a hyperbolic increase in the BRET signal (ratio of emission at 510/410 nm) as the ratio of GFP-receptor/RLuc-receptor increased, whereas nonspecific interactions increased linearly. Data were analyzed with Prism 6 (GraphPad, San Diego, Calif.) using best model (hyperbolic versus linear) for each experiment. GFP expression was determined by excitation at 495 nm and emission at 515 nm. Luciferase expression was determined by adding 5 mm coelenterazine H (Invitrogen, Carlsbad, Calif.) and reading total light emission without a filter. Each BRET assay was performed in three independent experiments.

TABLE 1
Major Resources

Animals (in vivo studies)

| Species | Vendor or source | Background strain | Sex |
|---|---|---|---|
| Mouse | Jackson Laboratories, | C57BL/6 | M/F |
| | Bar Harbor, ME | | |
| Mouse | C. Gerard, Children's | C57BL/6, C5ar1^{-/-} | M/F |
| | Hospital, Boston, MA | | |
| Mouse | Jackson Laboratories | PAR1^{-/-} | M/F |
| Mouse | Mutant Mouse Resource | PAR3^{-/-}PAR4^{-/-} | M/F |
| | & Research Centers, | | |
| | University of | | |
| | California, Davis, CA | | |

Animal breeding

| | | | Background | Other |
|---|---|---|---|---|
| Parent | Species | Vendor or source | strain | information |
| M | Mouse | Jackson | C57BL/6 | |
| | | Laboratories | | |
| F | Mouse | Jackson | C57BL/6 | |
| | | Laboratories | | |
| M | Mouse | C. Gerard | BALB/c, | Crossbred for |
| | | | C5ar1^{-/-} | 12 generations |
| F | Mouse | C. Gerard | C57BL/6 | Crossbred for |
| | | | | 12 generations |
| M | Mouse | C. Gerard | C57BL/6, | |
| | | | C5ar1^{-/-} | |
| F | Mouse | C. Gerard | C57BL/6, | |
| | | | C5ar1^{-/-} | |
| M | Mouse | Mutant Mouse | C57BL/6, | |
| | | Resource & | PAR1^{-/-} | |
| | | Research Centers | | |
| F | Mouse | Mutant Mouse | C57BL/6, | |
| | | Resource & | PAR1^{-/-} | |
| | | Research Centers | | |
| M | Mouse | Mutant Mouse | C57BL/6, | |
| | | Resource & | PAR3^{-/-}, | |
| | | Research Centers | PAR4^{-/-} | |
| F | Mouse | Mutant Mouse | C57BL/6, | |
| | | Resource & | PAR3^{-/-}, | |
| | | Research Centers | PAR4^{-/-} | |

Antibodies

| | | | | | Lot no. |
|---|---|---|---|---|---|
| | | | Working | | (preferred but |
| Target antigen | Vendor or source | Catalog no. | concentration | | not required) |
| Human/mouse FII | ThermoFisher, | PA1-43040 | 1 | μmol/mL | SI2444095 |
| | Carlsbad, CA | | | | |
| Mouse FV | Bioss, Woburn, MA | Bs-1040R | 5 | μg/mL | AD121701 |
| Mouse FVII | Bioss | Bs-4846R | 5 | μg/mL | YE1022W |
| Mouse FX | Bioss | Bs-9501R | 5 | μg/mL | 9B26M7 |
| Human/mouse | Santa Cruz | SC-53795 PE | 2 | μg/mL | C1810 |
| C5aR | Biotechnologies, Dallas, | | | | |
| | TX | | | | |
| Mouse C5a | BD Biosciences, | 558027 | 5 | μg/mL | |
| | Franklin Lakes, NJ | | | | |
| Human C5a | R&D Systems, | AF2037 | 5 | μg/mL | |
| | Minneapolis, MN | | | | |
| Human thrombin | ThermoFisher | HYB 109-04-02 | 5 | μg/mL | UI2843443 |
| Mouse/human | BD Biosciences | 611522 | 5 | μg/mL | 6013888 |
| PAR1 | | | | | |
| Mouse/human | Abcam, Cambridge, UK | Ab5787 | 5 | μg/mL | GR267332-5 |
| PAR4 | | | | | |
| Human/mouse | Cell Signalling | 2479 | 0.1 | μg/mL | 18 |
| VEGFR2 | Technologies, Danvers, | | | | |
| | MA | | | | |

Reagents

| | | | | | Lot no. |
|---|---|---|---|---|---|
| | | | Working | | (preferred but |
| Reagent | Vendor or source | Catalog no. | concentration | | not required) |
| C5a receptor | Calbiochem, Torrey | 234415 | 10 | ng/mL | |
| antagonist, | Pines, CA | | | | |
| W-54011 | | | | | |
| Vorapaxar (PAR1 | Axon Medchem, | 1755 | Batch no. 2 | | |
| antagonist) | Reston, VA | | | | |
| TFLLRN (PAR1 | Tocris Biosciences, | 1464 | 0.01-100 | ng/mL | |
| agonist) | Minneapolis, MN | | | | |
| AY-NH₂ (PAR4 | Tocris Biosciences | 1487 | 0.001-10 | ng/mL | 10A |
| agonist) | | | | | |
| Bivalirudin | Sigma, St. Louis, MO | SML1051 | 25 | μg/mL | 064M4731V |
| trifluoroacetate salt | | | | | |
| AS252424 (PI-3Kγ | Tocris Biosciences | 3671 | 10 | μmol/mL | 2A/153705 |
| inhibitor) | | | | | |
| SU5416 (VEGFR2 | Tocris Biosciences | 3037 | 10 | μmol/mL | |
| antagonist) | | | | | |

Human plasma

| Plasma | Source |
|---|---|
| Plasma deficient in | George King |
| FII | Biomedical, Overland |
| | Park, KS |
| Plasma deficient in | George King |
| FV | Biomedical |
| Plasma deficient in | George King |
| FVII | Biomedical |
| Plasma deficient in | George King |
| FX | Biomedical |

siRNA

| | | | | Working |
|---|---|---|---|---|
| | | | | concentration, |
| Target | Vendor or source | Catalog no. | ID | nmol/L |
| Human PAR1 | ThermoFisher | AM16708 | 146259 | 30 |
| Human PAR4 | ThermoFisher | 4392420 | s194972 | 30 |

Cultured cells

| | | Sex (F, M, or |
|---|---|---|
| Name | Vendor or source | unknown) |
| bEnd.3 | ATCC, Manassas, VA | Unknown |
| Primary aortic | ATCC | Unknown |
| endothelial cells; | | |
| normal, human | | |
| (HAECs) | | |
| HUV-EC-C | ATCC | Unknown |
| (HUVECs) | | |
| 293 (HEK-293) | ATCC | Unknown |

F, female; M, male; C5ar1, C5a receptor-1; FII, factor II; FV, factor V; FVII, factor VII; FX, factor X; HAEC, human aortic endothelial cell; HUVEC, human umbilical vein endothelial cell; ID, identifier; PAR, protease-activated receptor; VEGFR2, vascular endothelial cell growth factor receptor 2.

### RNA Interference

siRNAs targeting human PAR1 (identifier: 146259) and PAR4 (identifier: s194972) were purchased from Thermo-Fisher (Carlsbad, Calif.). RNA interference was performed using Lipofectamine RNAiMAX Reagent (ThermoFisher) following the manufacturer's recommended protocol. Briefly, cells were treated with 30 nmol/L siRNA and transfection reagent at a 1:2 ratio under reduced serum conditions for 48 hours. Cells were returned to complete media, and knockdown was assessed using flow cytometry at 3 and 7 days. Silencer-Cy3 siRNA (ThermoFisher) was used as a nonspecific control.

### Statistical Analysis

Power calculations and animal numbers were determined using the information provided by http://statpages.org and the therein linked Russ Lenth's power and sample-size calculator obtainable through http://www.stat.uiowa.edu/wrlenth/Power/index. html#Download_to_run_locally (both last accessed Sep. 12, 2021). To achieve a true difference between means of 0.5 and a power of 0.2 testing the difference between two means via an unpaired, two-tailed t-test, 10 mice were required in each group and there were 10 experimental groups. Statistical significance for all experimental data was determined by t-test (unpaired, two tailed) performed in Microsoft Excel (Seattle, Wash.), SigmaPlot (Systat Software, Chicago, Ill.), or GraphPad Prism 6, with a significance threshold value of P<0.05. Except where indicated, all experiments were repeated at least two times in separate samples. Data are presented as mean values with SD.

### Results

### ECs Synthesize Coagulation Factors, and the Synthesis is Up-Regulated by VEGF-A

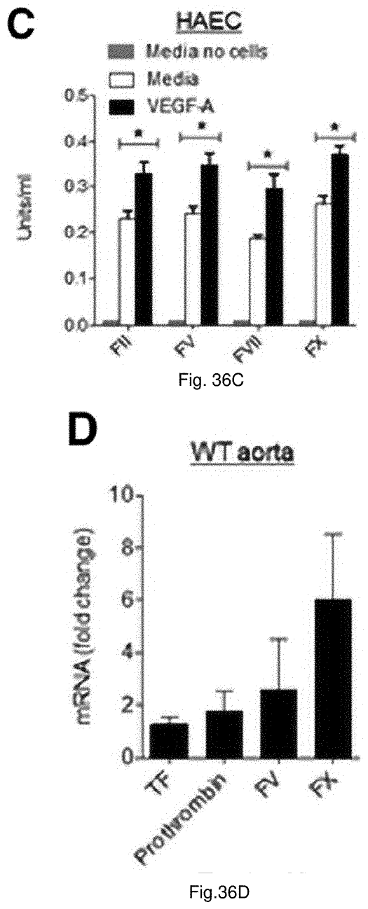

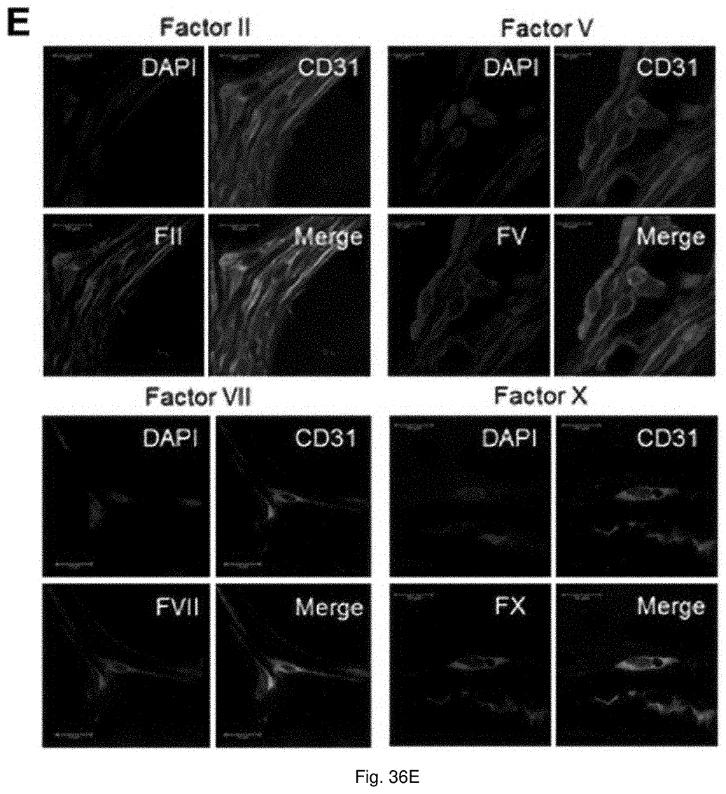

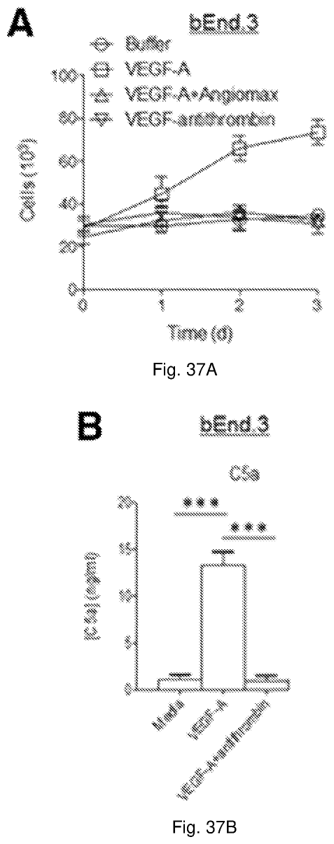

To test whether ECs endogenously synthesize coagulation factors and, if so, whether VEGF-A stimulation affects their synthesis, a culture system with noncontact inhibited ECs was used to model EC growth as occurs in blood vessel reconstitution. Initial cell numbers were plated such that cells would remain <70% confluent in the presence of VEGF-A at the longest incubation time. In initial studies, murine bEnd.3 ECs incubated for 1 hour were used in the absence or presence of added VEGF-A. Immediately after harvesting the ECs, the washed cells were assayed for coagulation factor mRNA transcripts. The cells incubated with VEGF-A showed 350% to 500% increased amounts of FV, FX, FVII, and FII (prothrombin) mRNAs adjusted to fold increases over media alone (FIG. 36A). Whether the mRNA synthesis translates to protein synthesis by measuring coagulation factors in culture supernatants was assessed next. Supernatants of the cells incubated in media alone shortened the clotting times of plasma samples selectively deficient in FII, FV, FVII, and FX (FIG. 36B), whereas those of VEGF-A-treated cells more profoundly shortened the clotting times of each (FIG. 36B). Primary aortic endothelial cells (HAECs) were used to document that the findings apply to nontransformed primary cells and human ECs. Incubation of HAECs without or with human VEGF-A showed comparable coagulation factor mRNA production (FIG. 36C). These data point to endogenous production of functional FV, FX, FVII, and FII by ECs tonically and augmented production of each in response to VEGF-A stimulation.

As a first test of whether the EC coagulation factor synthesis occurs in vivo, FV, FX, FVII, and FII production was examined in thoroughly perfused aortae from WT mice. Real-time quantitative PCR of dispase extracts were used to documente FII, FV, and FX as well as tissue factor mRNA expression (FIG. 36D). Because aortae contain other cell types (e.g., smooth muscle cells), and aortic ECs in situ do not proliferate (under homeostatic conditions), Matrigel plugs were implanted into the flanks of WT mice, plugs were removed 10 days later, and blood vessel growth was examined for intracellular coagulation factors. Confocal microscopy showed abundant FV, FX, FVII, and FII in EC cell bodies that colocalized with the EC marker CD31 (FIG. 36E). These studies support the concept that the EC coagulation protein synthesis operates in vivo in proliferating ECs.

### Thrombin Generation of C5a in ECs is Integral to VEGF-A Mitotic Function

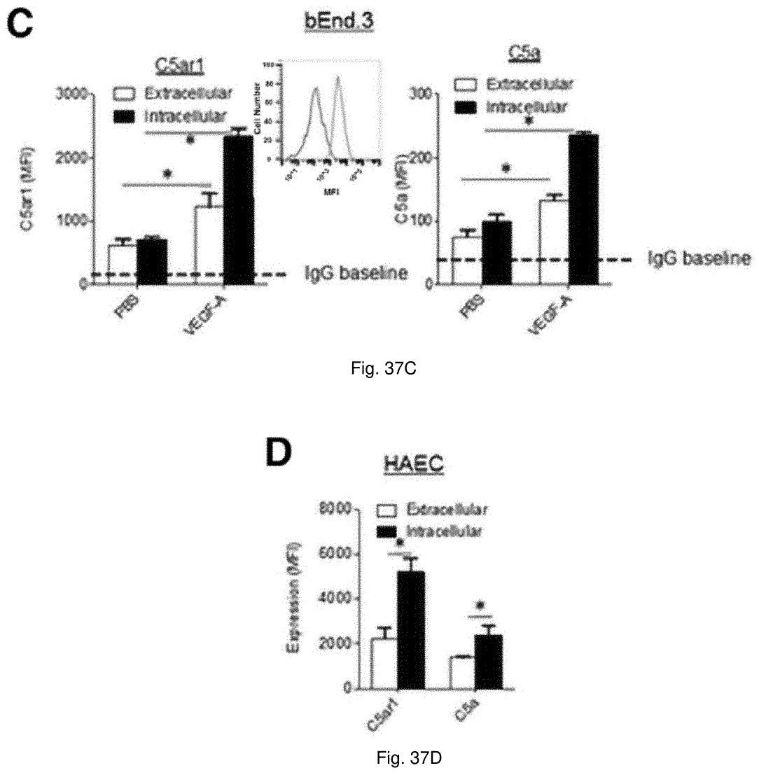

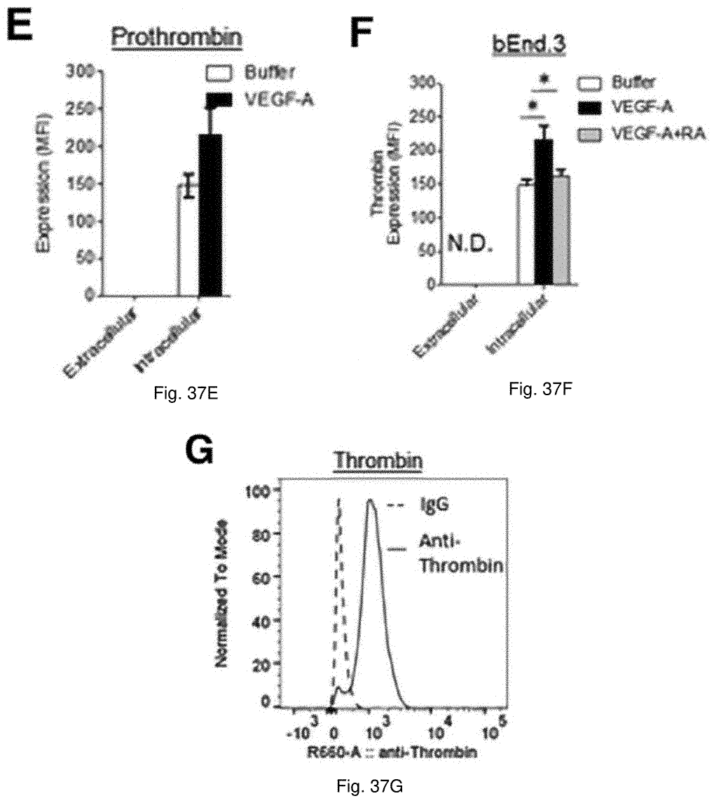

Whether EC coagulation factor production generates EC thrombin and, if so, whether the endogenously produced thrombin is mechanistically interconnected with VEGF-A mitotic function was tested next. Bivalirudin (Angiomax) or antithrombin (two different thrombin-specific inhibitors) was added to (nonconfluent) growing cultures of bEnd.3 cells in the absence or presence of added VEGF-A and EC proliferation was assessed over 72 hours. Consistent with the EC coagulation factor production being mechanistically tied to EC growth, both inhibitors completely abolished VEGF-A's induction of EC proliferation (FIG. 37A). The thrombin active site inhibitors PPACK and FM19 had a similar abrogating effect (data not shown), supporting an essential role of EC coagulation factor synthesis in VEGF-A function. Taken together with the recent findings that EC-produced C5a plays a requisite role in EC growth, these data point to VEGF-A mitotic function, depending on activation of both EC-produced complement components and coagulation factors.

The earlier studies of the neo-intimal response to femoral artery wounding implicated C5ar1 signaling in the proliferation of ECs into wounds but presumed that the C5a activating ligand was derived from plasma C5. On the basis of these earlier findings, the above thrombin blockade data herein, and the earlier report that thrombin can cleave C5a from purified C5 in vitro, we hypothesized that C5a in the setting of EC growth would derive from cleavage of EC produced C5 by EC produced thrombin. To test this idea, proliferating (nonconfluent) bEnd.3 cells were cultured with VEGF-A with or without antithrombin and the cells and their culture supernatants were assayed for C5a. Abundant C5a was generated in cultures with VEGF-A alone, whereas little, if any, was generated in the presence of thrombin blockade by antithrombin (FIG. 37B). To directly determine whether VEGF-A augments thrombin-induced C5a production and determine whether the process is localized within ECs, unstimulated and VEGF-Aestimulated bEnd.3 cells were assayed for C5a without or with permeabilization of the cells. VEGF-A stimulation augmented the production of C5a as well as its C5ar1 receptor; and EC permeabilization showed that both augmentations occurred predominantly intracellularly within the ECs (FIG. 37C). To verify that the findings apply to primary cells and human ECs, studies were repeated with HAECs. Comparable results were obtained (FIG. 37D). Similar to that in the EC-produced C5a deriving from thrombin cleavage of EC-produced C5, added VEGF-A concomitantly augmented a prothrombin/thrombin signal intracellularly in the ECs (FIG. 37E). The elicited pro-thrombin/thrombin signal was blunted by pharmaceutical blockade of C5ar1 (FIG. 37F) interconnecting EC thrombin generation and EC C5a production. These results are consistent with the previous studies connecting VEGF-A induction of EC growth with up-regulation of autocrine C5ar1 signaling occurring intracellularly in endosomes.

To exclude the possibility that the thrombin-induced cleavage of C5a in ECs resulted from prothrombin contained in the bovine serum-supplemented medium and document that the intracellular prothrombin/thrombin signal induced by VEGF-A contained thrombin, the primary HAECs were cultured in serum-free medium for 2 days. Intracellular fluorescence-activated cell sorting of the serum-free cells with a thrombin-specific monoclonal Ab (mAb; that does recognize prothrombin) showed a homogeneously positive anti-thrombin signal (FIG. 37G). These data linking VEGF-A with intracellular generation of both thrombin and C5a taken together with the finding that antithrombin blocked the EC C5a production (FIG. 37G) pointed to C5a being generated by EC produced thrombin, an issue that will be further examined below.

Locally Produced Thrombin Induces Cooperative C5ar1 and PAR Signaling that Promotes EC Proliferation

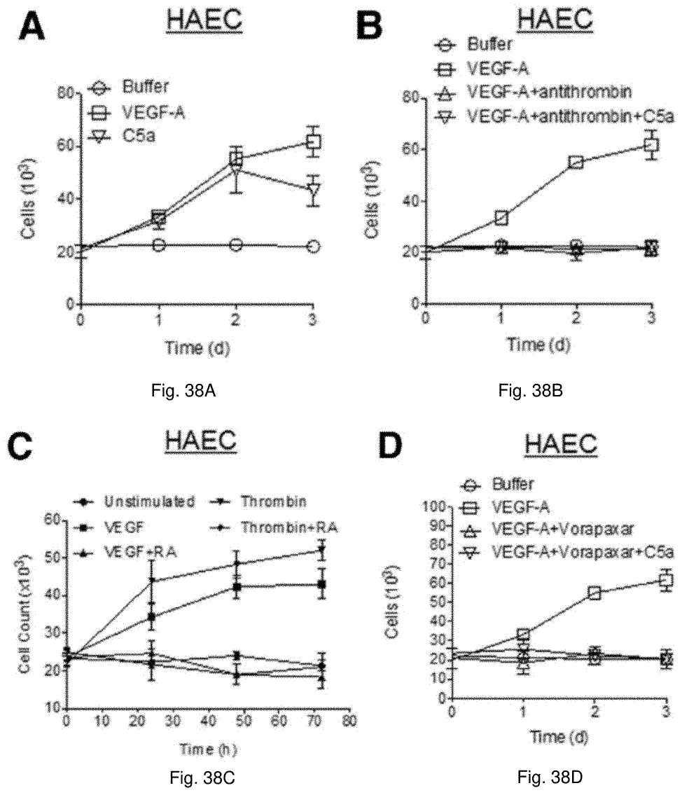

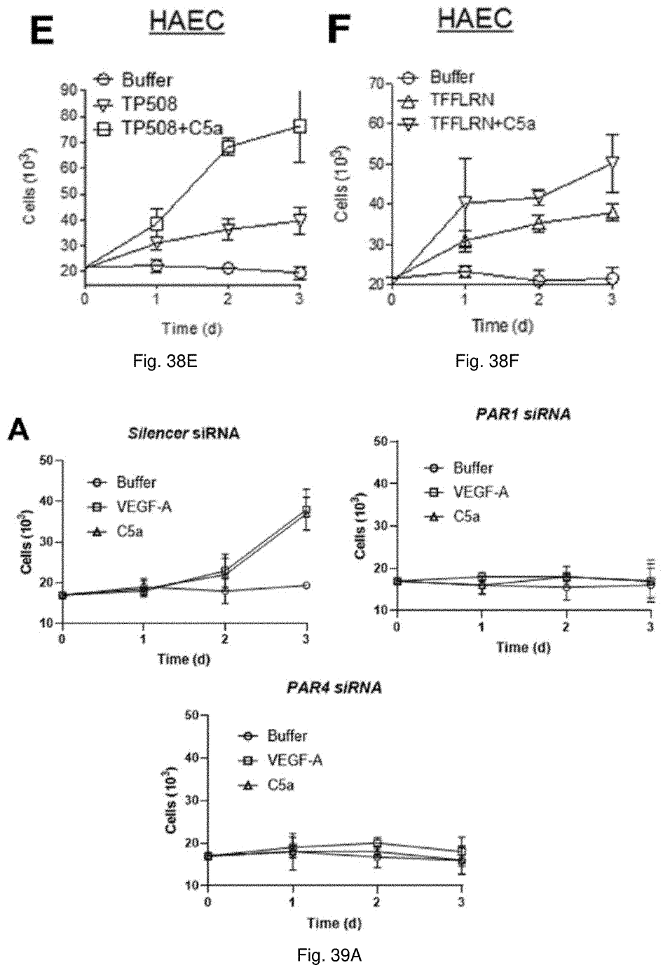

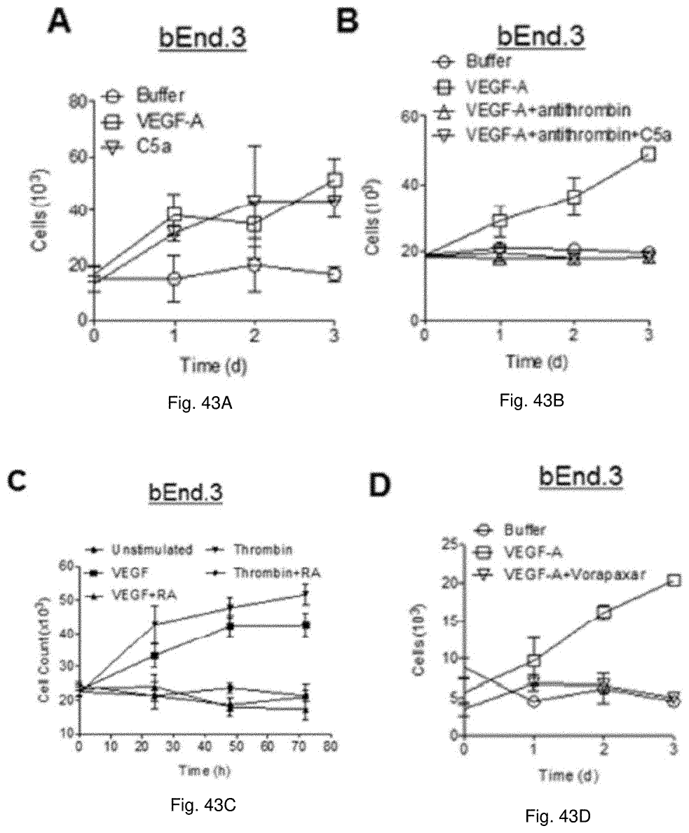

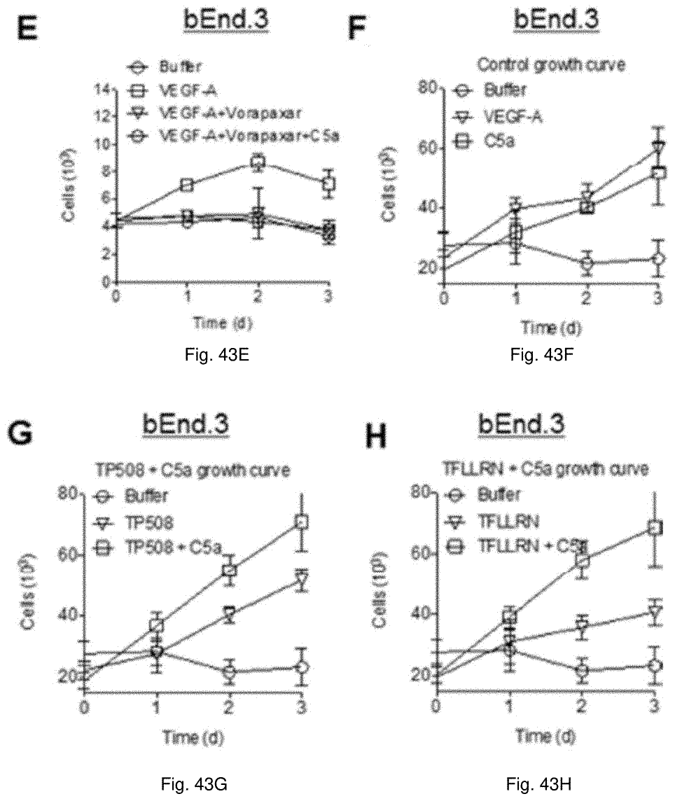

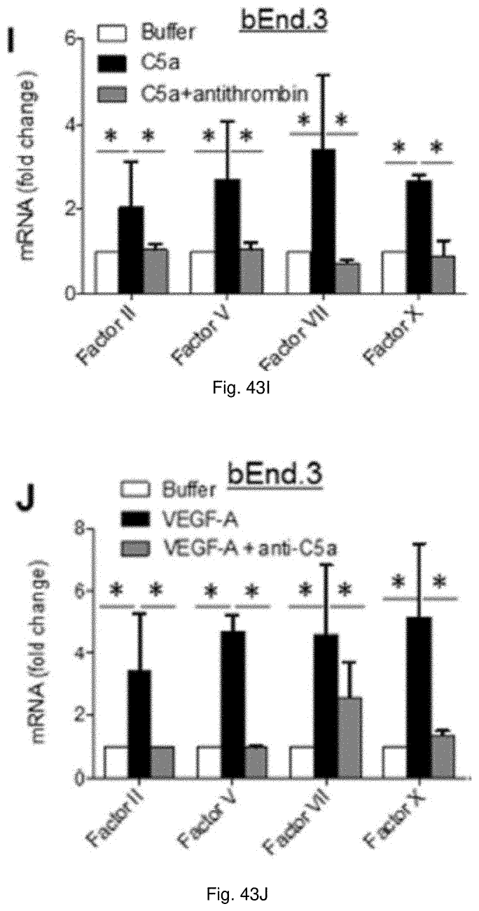

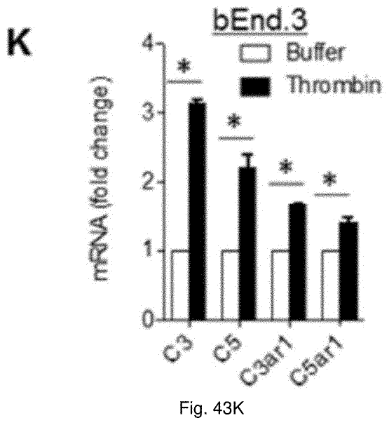

The generation of both thrombin and C5a intracellularly in proliferating ECs raised the question of whether VEGF-A induction of EC growth is dependent on the heightened generation of both. Adding C5a to primary cultures of HAEC (or to murine bEnd.3 cells) induced cell growth similar to adding VEGF-A (FIG. 38A and FIGS. 43A, E, and F). On the other hand, VEGF-A failed to induce growth when thrombin generation was blocked with antithrombin, even if C5a was included in the cultures (FIG. 38B and FIG. 43B). Adding thrombin, like adding C5a, similarly induced the growth of both primary HAEC and bEnd.3 cells with efficiency comparable to adding VEGF-A (FIG. 37C), whereas thrombin failed to do so if C5ar1 signaling was blocked by C5ar1 pharmaceutical antagonism (RA:C3ar1/C5ar1 antagonists) (FIG. 38C and FIG. 43C). The findings that C5a and thrombin induced EC proliferation only in the presence of each other pointed to a mechanism in which both are jointly involved in EC growth. Neither by itself was sufficient for optimal VEGF-A induction of EC growth.

The failure of VEGF-A to induce EC growth in the presence of thrombin blockade, despite supplying its C5a product, raised the question of whether thrombin additionally provides for VEGF-A mitotic signaling via signaling through EC expressed PAR(s). As a first test of this idea, bEnd.3 cells (which express PAR1 and PAR4) were incubated with VEGF-A with or without vorapaxar, a drug known to inhibit human PAR1 signaling. The added vorapaxar abolished VEGF-A induction of (murine) bEnd.3 cell growth (FIG. 43D), as well as primary (human) HAEC growth (FIG. 38D), irrespective of supplementing the cultures with excess C5a.

Seemingly inconsistent with the above findings, a prior study using confluent HUVECs found that thrombin induction of PAR1 signaling suppresses rather than promotes EC proliferation in response to epidermal growth factor and does so by disabling AKT phosphorylation. On the basis of the current data, we hypothesized that in proliferating ECs, in contrast to confluent HUVECs, which are contact inhibited, PAR signaling confers different signals. To test this concept, bEnd.3 cells and primary HAECs were cultured with TP508 (a 23eamino acid peptide from thrombin; i.e., a portion of the PAR1 receptor-binding domain of thrombin) or with TFLLRN (an agonist peptide corresponding to the peptide sequence of the tethered PAR1 ligand). Each agonist was added individually, or in combination with C5a, to primary HAEC or bEnd.3 cells and EC proliferation was assessed. Although each PAR1 agonist alone induced primary HAEC (FIGS. 38E and F) and bEnd.3 cell growth (FIGS. 43G and H), the inclusion of C5a had an additive effect. These findings argue that in growing ECs, PAR1 signaling augments rather than suppresses growth and that it coordinates with C5ar1 signaling in promoting EC growth. Additional studies comparing growing and confluent HAECs are discussed below.

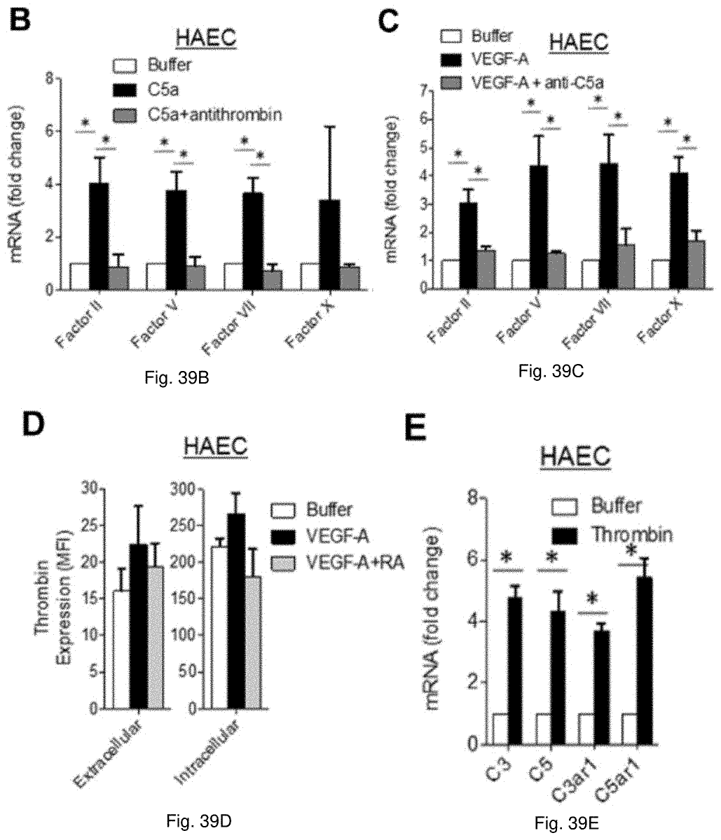

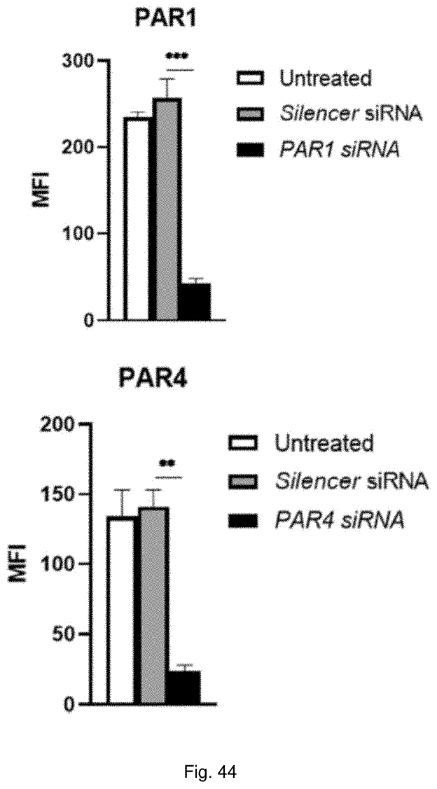

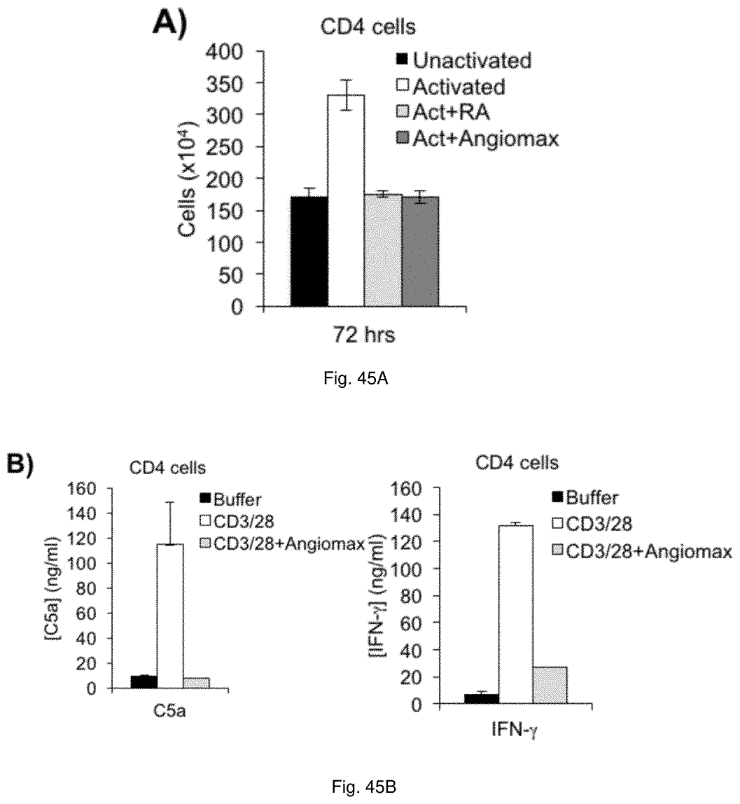

Vorapaxar not only inhibits PAR1 signaling but also can impact PAR4 signaling as it makes heterodimers with PAR4 that coordinate in platelet activation in a time-dependent manner. siRNA studies were performed to determine whether thrombin's induction of EC growth involves PAR4 as well as PAR1 signaling and establish whether the activation of both is required for VEGF-A mitotic function. To exclude thrombin in serum, serum-free conditions were used. HAECs were treated with siRNAs targeting PAR1 or PAR4, or with Silencer (non-specific) siRNA control. Flow cytometry verified that the specific siRNAs fully abolished PAR1 and PAR4 cell surface expression (FIG. 44). The alternatively treated HAECs were first pre-incubated in serum-free medium for 16 hours, and then VEGF-A induced growth was examined over an additional 72 hours, maintaining the HAECs in serum-free medium. In contrast to the Silencer siRNA treated HAECs, which robustly grew in response to either VEGF-A or C5a (FIG. 39A), the HAECs silenced in either PAR1 or PAR4 showed no growth when incubated with either agonist (FIG. 39A).

### EC-Produced Thrombin Modulates EC C5a Production and Vice Versa

The codependence of VEGF-A mitotic function on both endogenous thrombin and C5a prompted studies of whether augmented complement component and coagulation factor production induced by VEGF-A regulate each other. Adding C5a to cultures of HAEC or of bEnd.3 cells up-regulated mRNA expression of all of the EC produced coagulation proteins (FIG. 39B and FIG. 43I), and anti-thrombin inhibited all of the up-regulations (FIG. 39B and FIG. 43I). Conversely, disrupting C5ar1 signaling with a (blocking) anti-C5a mAb prevented VEGF-A's up-regulation of coagulation factor mRNAs (FIG. 39C and FIG. 43J). Consistent with this, the C5ar1 blockade repressed VEGF-A's induction of intracellular pro-thrombin/thrombin generation (FIG. 39D and FIG. 43K) (the intracellular ordinate axis is 10-fold higher than that of the extracellular ordinate axis). These data indicate that coagulation factor synthesis depends on EC C5ar1 signaling. In accordance with the mRNA expression interdependence occurring reciprocally, added thrombin up-regulated EC complement component mRNA expression (FIG. 39E and FIG. 43K).

These findings point to VEGF-A's induction of autocrine PAR signaling and autocrine C5ar1 signaling occurring coordinately in ECs. The findings also point to the two systems regulating each other by virtue of thrombin being needed for generation of C5a, and C5ar1 signaling being needed for coagulation protein synthesis (FIGS. 39, C and D). Taken together, these data indicate that endogenous complement production in ECs up-regulates endogenous prothrombinase production and vice versa and that both up-regulations play requisite roles in VEGF-A mitotic signaling.

### C5ar1 and PAR4 are Associated, and Their Joint Signaling is Interconnected in ECs

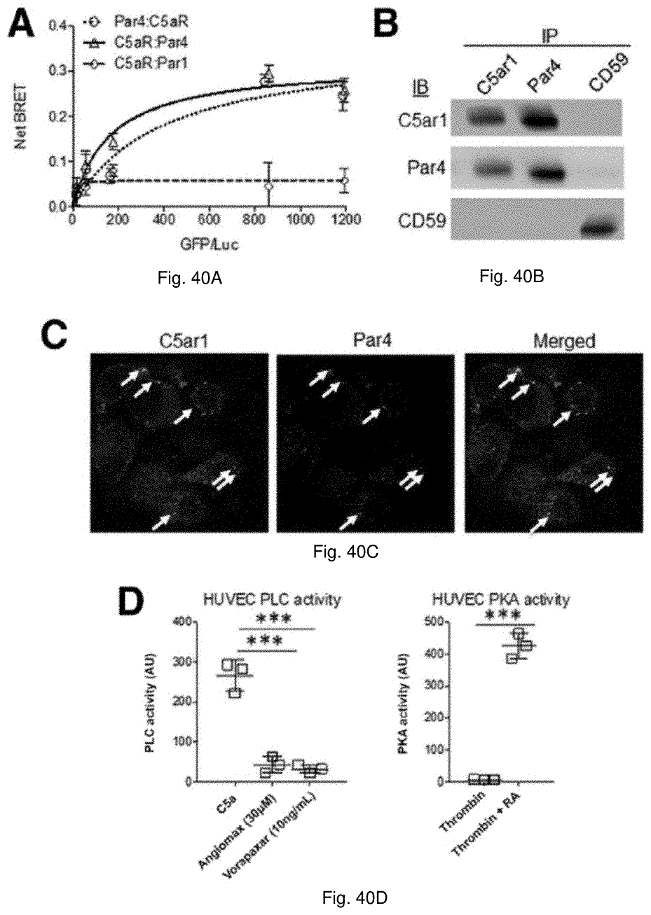

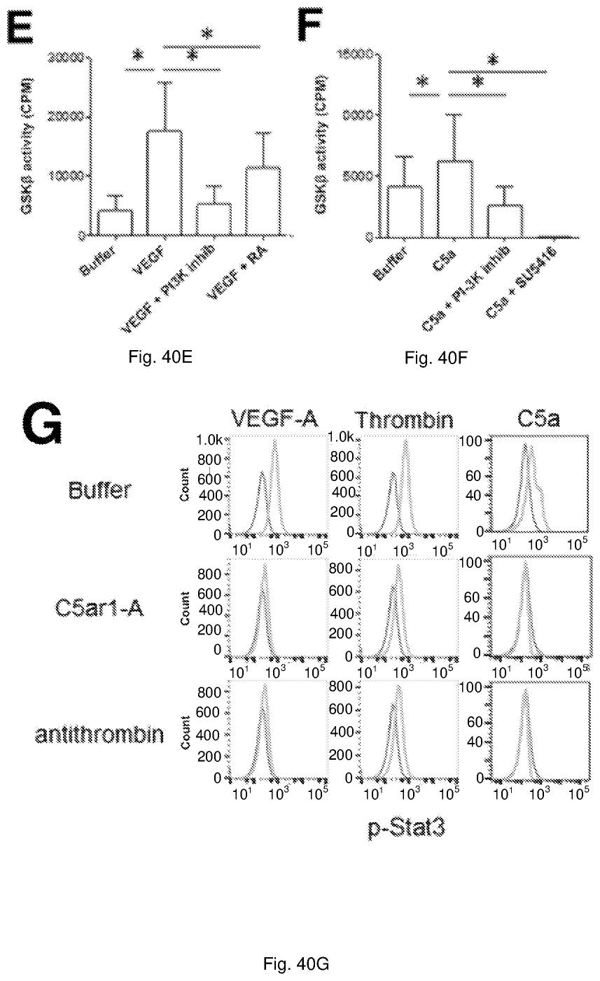

Because locally produced thrombin in ECs elicited joint C5ar1 and PAR signaling, we hypothesized that PAR1, PAR4, or both and C5ar1 might be in proximate contact. To test this idea, BRET analyses were done first. HEK293 (human kidney epithelial cells) cells were cotransfected with C5ar1-GFP together with PAR1-luciferase or with PAR4-luciferase. There was a dose-dependent energy transfer in the cells containing C5ar1 plus PAR4-luciferase (FIG. 40A), but not PAR1-luciferase (FIG. 40A). A reverse analysis with C5ar1-luciferase and PAR4-GFP yielded the same positive result (FIG. 40A). These data indicate that C5ar1 and PAR4 are within 10 nm of each other in membranes. Co-immunoprecipitation assays were performed next. In support of the BRET data, anti-C5ar1 co-immunoprecipitated PAR4 and vice versa (FIG. 40B), but PAR1 was not brought down in either immunoprecipitation (FIG. 40B). Finally, confocal imaging with GFP-labeled anti-C5ar1 mAb and Red Fluorescent Protein (RFP)-labeled anti-PAR4 mAb showed colocalization of the two receptors (FIG. 40C). These results pointed to a functionally interactive complex between PAR4 and C5ar1, an arrangement that could facilitate thrombin's ability to concurrently produce the C5ar1 and PAR4 ligands and coordinately elicit signaling by the two receptors.

Because C5ar1 and PAR4 are both GPCRs, their association raised the possibility of the existence of additional signaling interconnections. C5ar1 [as indicated (Introduction)], is a Gi coupled GPCR such that a subunit of its G protein represses activation of adenylyl cyclase. PAR4 is (predominantly) a Gq coupled GPCR such that its G protein a subunit activates phospholipase C (PLC). Some past studies have reported that C5ar1 can couple to Gaq as well as Gai. Others have reported that PAR4 can couple to Gai as well as Gaq. To determine whether combinatorial C5ar1-PAR signaling might reconcile these observations, HUVECs were incubated with C5a alone, together with Angiomax (which blocks thrombin), or together with vorapaxar (which blocks PAR1/PAR4 signaling) (FIG. 38D) and PLC activation was assayed. C5a activation of the Gaq target PLC occurred when C5a was added alone (FIG. 40D), but the activation was reversed by Angiomax that blocks thrombin needed for PAR as well as C5ar1 signaling. Notably, vorapaxar (which blocks PAR1-dependent PAR4 signaling) (FIG. 38D) had the same effect (FIG. 40D). In the case of the Gai signaling of PAR4, there have been reports that in platelets PAR4 can couple to the purinergic Gai GPCR P2Y12. Nevertheless, although thrombin suppressed protein kinase A activation (a result of suppressed generation of the Gai target adenylyl cyclase) in primary HAECs, it did not do so in the presence of C3arl/C5ar1 pharmaceutical antagonists (RA) implicating C5ar1 signaling (FIG. 40D). These data provide evidence that PLC can be activated by C5ar1 signaling and adenylyl cyclase can be repressed by PAR4 signaling. They argue that the cross-activation can occur by virtue of C5ar1 and PAR4 being functionally associated with each other in primary HAECs and the agonist of either GPCR being able to activate the other GPCR.

Disruption of C5ar1 or PAR Transduction Blocks AKTeGSK-beSTAT3 and AKT-Mammalian Target of Rapamycin 2 (mTOR) Signaling

VEGF-A induction of EC proliferation is dependent on activated STAT3. Previous studies in immune cells and recent studies in ECs showed that C5ar1 signaling promotes STAT3 activation. To determine whether, and how, combinatorial EC C5ar1 and PAR1/4 signaling participates in STAT3 activation, VEGF-A induction of AKTeGSK-beSTAT3 signaling in ECs was examined. Addition of C5a, like that of VEGF-A (FIG. 38A), to cultures of bEnd.3 cells evoked S⁴⁷³ phosphorylation of AKT 14, activation of GSK-b (FIGS. 40E and F), and Y705 phosphorylation of STAT3 (FIG. 40G). The activation of GSK-b by VEGF-A was suppressed by C5ar1 blockade (FIG. 40E) and that by C5a was reversed by VEGFR2 blockade (FIG. 40F). STAT3 activation by VEGF-A was suppressed by either C5ar1 or thrombin blockade, and that by thrombin was suppressed by C5ar1 blockade (FIG. 40G), consistent with both PAR4 and C5ar1 signaling being essential. GSK-b activation by C5a was abolished by the VEGFR2 inhibitor SU5416 (FIG. 40F). GSK-b activation by VEGF-A was abolished by the AS252424 inhibitor of phosphatidylinositol 3-kinase gamma (PI-3Kg) (FIGS. 40E and F) (i.e., downstream of both C5ar1 and PAR4 transduction and possibly related to either thrombin or C5a being able to activate both C5ar1 and PAR4). Collectively, these data argue that thrombin as well as C5ar1 signaling are needed for VEGFR2-associated induction of GSK-b and STAT3 activation. The data further argue that AKTeGSK-beSTAT3 signaling is dependent on PI-3Kγ activation.

### Both C5ar1 and PAR4 Signaling Promote VEGFR2 Signaling in ECs

How joint C5ar1 and PAR1/4 signaling in ECs affects VEGF-A induction of the PI-3KeAKTemTOR cascade was investigated next. Prior studies in CD4 [symbol] T cells showed that C5ar1 activation of PI-3Kγ is essential for augmented PI-3KeAKTemTOR signaling. In distinction to this linkage, early studies of CD4 [symbol] cell activation had linked costimulatory CD28-dependent activation of PI-3Ka with AKT-mTOR signaling. Taken together, the two results suggest that both activated PI-3K isoforms [that jointly promote phosphatidylinositol 3,4,5 trisphosphate (PIP3) assembly] may be needed for AKT-mTOR signaling in ECs as in T cells.

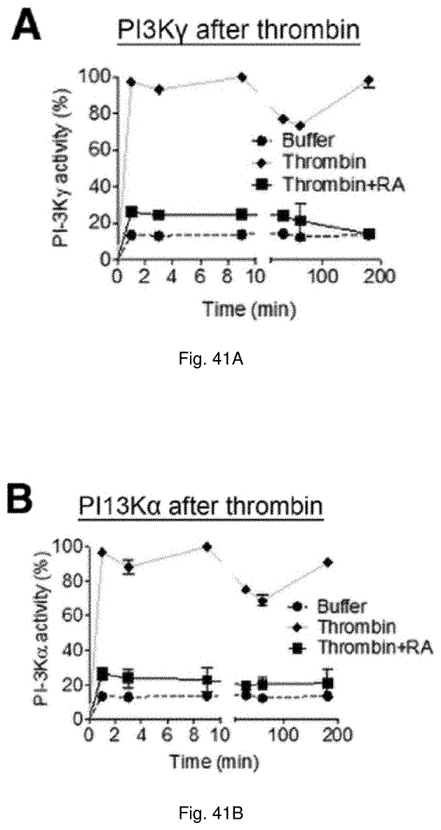

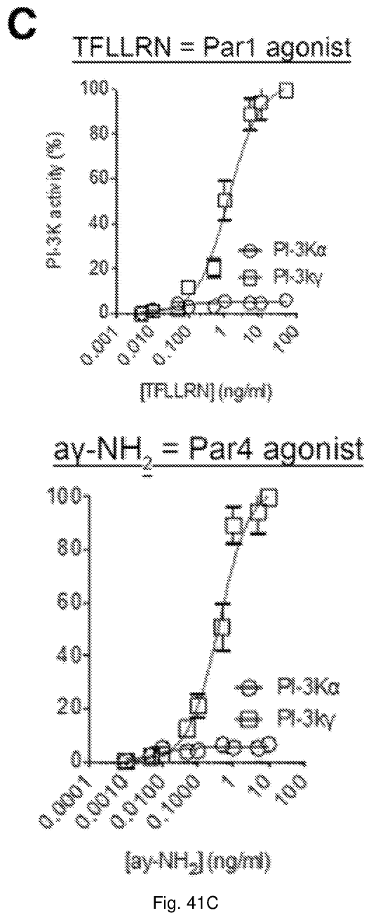

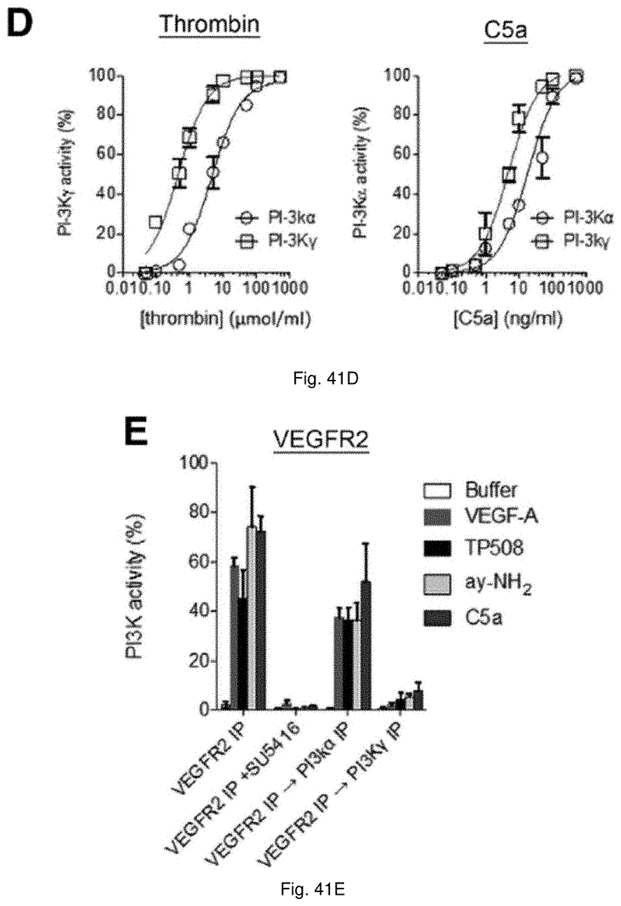

To characterize the respective roles of C5ar1 and of PAR4 signaling in PI-3Kγ and of PI-3Ka activation in the context of EC proliferation, bEnd.3 ECs were first incubated with thrombin and the activation of each PI-3K isoform was assayed. The added thrombin (which jointly induces both PAR4 and C5ar1 signaling) activated both PI-3K isoforms with high efficiency (FIGS. 41A and B). The activation of both isoforms occurred in <1 minute and was sustained for >180 minutes (FIGS. 41A and B). The activation of both isoforms was abolished by C5ar1 antagonism, highlighting C5ar1 signaling as an obligate process in thrombin-induced activation of the two PI-3K isoforms (FIGS. 41A and B). To distinguish the roles of PAR1 and PAR4 in activating each PI-3K isoform, PAR agonists and antagonists were used. Titrations of agonists specific for PAR1 and PAR4 into cultures of (nonactivated) bEnd.3 cells showed that signaling by either PAR activated PI-3Kγ but did not activate PI-3Ka (FIG. 41C). This contrasted with titrations of C5a into the culture, which activated both PI-3Kγ and PI-3Ka, localizing the PI-3Ka activating effect of thrombin (which jointly induces PAR and C5ar1 signaling) to C5ar1 signaling (FIG. 41D). Dose-response curves of either thrombin or C5a addition to bEnd.3 cells indicated that activation of PI-3Kγ preceded that of PI-3Ka. This timing would be consistent with PI-3Kγ functioning upstream to repress phosphatase and tensin homolog (PTEN) restraint of PIP3-dependent AKT activation, and PI-3Ka (known to be VEGFR2-activated) being mechanistically involved in augmenting PIP3 generation.

Because PI-3Kγ activation occurs on membranes nearby GPCRs in contrast to PI-3Ka activation, which typically occurs on receptor tyrosine kinases like VEGRF2, VEGFR2 immunoprecipitations were examined for associated PI-3Ka following EC activation with the PAR1, PAR4, and C5ar1 agonists. bEnd.3 cells were incubated with TP508 (PAR1 agonist), ay-NH2 (PAR4 agonist), C5a, or VEGF-A, after which extracts of the activated cells were incubated with anti-VEGFR2 mAb. Twenty-four hours later, VEGFR2 was immunoprecipitated from each incubation mixture and immunoprecipitated proteins were eluted from the alternatively activated bEnd.3 cells. Secondary antiePI-3Kγ or PI-3Ka immunoprecipitations of the eluents of the anti-VEGFR2 immunoprecipitations were prepared from each incubation culture and the secondary antiePI-3Kγ immunoprecipitations and antiePI-3Ka immunoprecipitations were tested for PI-3K enzymatic activity. The antiePI-3Kγ immunoprecipitations from cells activated with each of the agonists uniformly lacked VEGFR2-associated PI-3Kγ activity (FIG. 41E). This would be in accordance with PI-3Kγ (due to its membrane localization near PIP3) not being immunoprecipitated by anti-VEGFR2 mAb. In contrast, the antiePI-3Ka immunoprecipitations from cells treated with all agonists uniformly contained PI-3Ka activity, which in all cases was abolished by the VEGFR2 antagonist SU5416. These data indicate that, in contrast to the short-term incubations, PAR1 or PAR4 signaling in ECs incubated overnight induced VEGFR2 signaling and its activation of PI-3Ka. In support of this interpretation, PI-3Ka activation by all agonists was knocked down in cells pretreated with VEGFR2 siRNA (data not shown). More studies in long-term cultures will be needed to determine whether PAR1/4 signaling transactivates directly to VEGFR2, as the recent studies have shown for C5ar1 signaling, whether it transactivates indirectly via the PAR4-C5ar1 complex characterized above, or whether another mechanism is involved.

Collectively, these data argue that PAR1, PAR4, and C5ar1 signaling all participate in VEGF-A's induction of EC growth. Although further studies are needed to directly validate the functional relationships and kinetics, the data are consistent with the following hypothetical formulation. They would be consistent with C5ar1 and PAR1 (because of its higher affinity than PAR4 for thrombin) initially collaborating in activating PI-3Kγ to repress PIP3 dephosphorylation. They support prior findings that C5ar1 directly transactivates to VEGFR2 to promote its signaling. They further support the concept that C5ar1 Gai signaling additionally functions to enable AKT and STAT3 activation and joint PAR4 Gaq signaling functions to enable PLC activation. The anti-VEGFR2 immunoprecipitation data following the addition to ECs of individual ligands of each receptor argue that all coordinately participate in VEGFR2-associated activation of PI-3Ka. According to the data presented herein, C5a and the PAR ligands could each induce combinatorial C5ar1 and PAR4 signaling. These results are in accordance with the vorapaxar blocking studies (FIG. 38), C5ar1-PAR4 association studies (FIG. 40), and the assays with PAR1 and PAR4 siRNAs (FIG. 39), which show that PAR1, PAR4, and C5ar1 all collaborate in enabling VEGF-A mitotic function.

Down-Regulated C5ar1 and PAR Signaling is Connected with Contact Inhibition

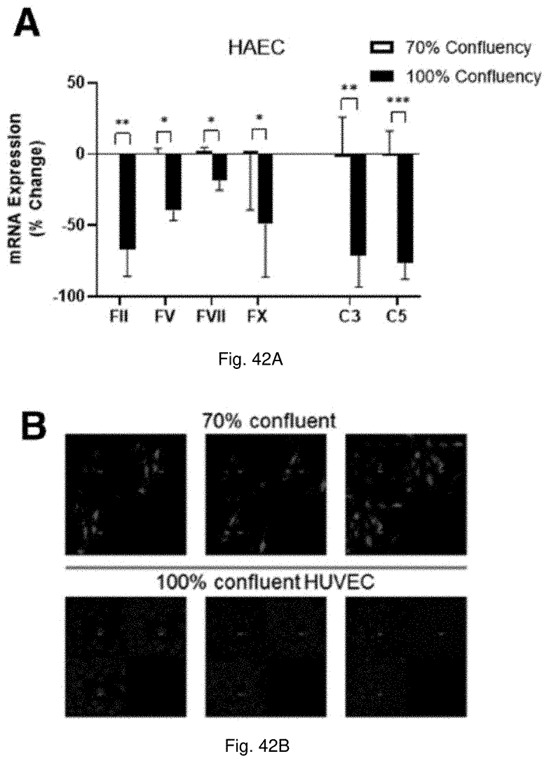

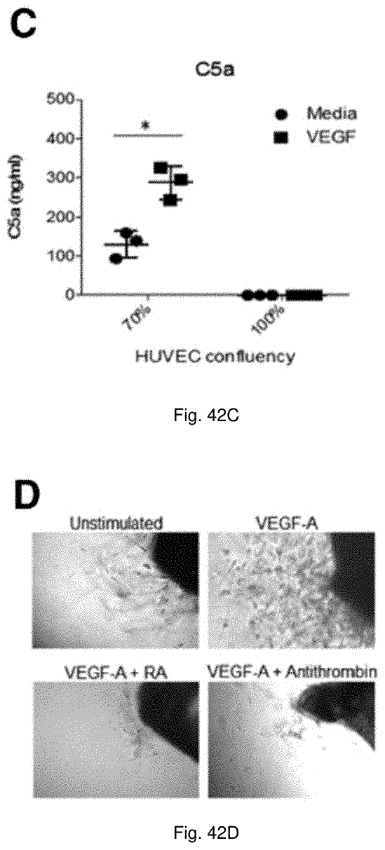

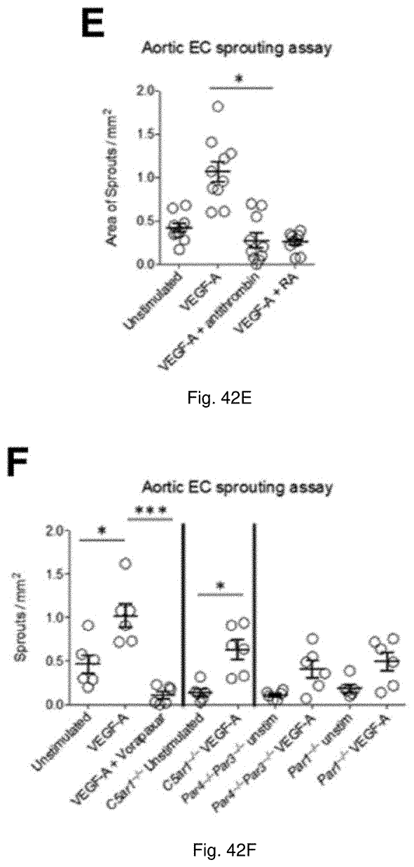

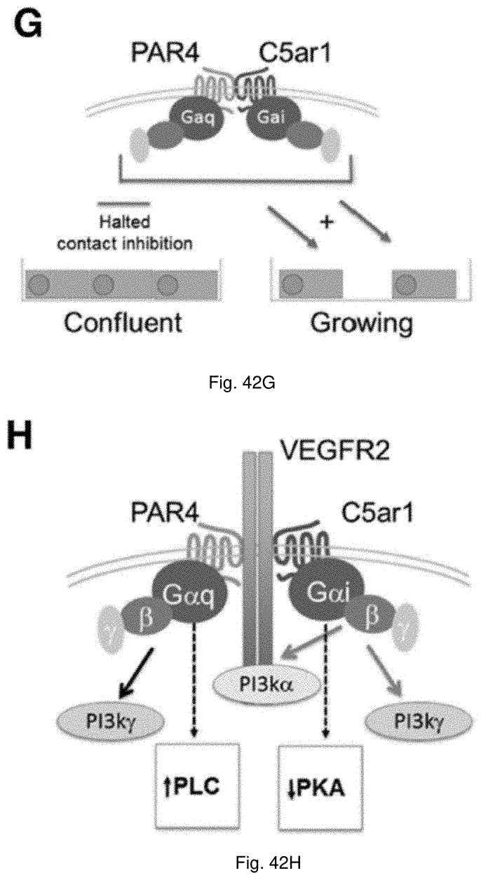

The experimental system using subconfluent ECs was chosen to model proliferating ECs (e.g., ECs in the context of neo-angiogenesis or reconstitution of vasculature following blood vessel severance). In this system, VEGF-A induces joint C5ar1 and PAR1/4 signaling which is integrally interconnected with VEGF-A mitotic function. Because the current studies of subconfluent ECs differ from most studies of ECs that use confluent HUVECs to model ECs in intact blood vessels, thrombin generation and C5a production in subconfluent growing HUVECs was compared to that in confluent HUVECs. Although subconfluent HUVECs expressed coagulation factor and complement component mRNAs, the mRNA expression of both systems down-regulated at confluence (FIG. 42A). In accordance with this result, the production of thrombin and of C5a was robust in growing HUVECs, whereas both were repressed in confluent (contact-inhibited) HUVECs (FIGS. 42B and C). These findings indicate that PAR-C5ar1 signaling operates in growing ECs and is repressed with contact inhibition.

### VEGF-A Induction of EC Sprouting Depends on Joint C5ar1 and PAR1-PAR4 Signaling

To validate that joint C5ar1 and PAR signaling is required for VEGF-A induction of EC growth physiologically, we performed sprouting assays. Aortic segments from WT mice incubated in Matrigel with VEGF-A showed robust sprouting compared with those from segments treated with media alone (1.07 versus 0.42 mm²; P<0.05) (FIG. 42D). Addition of C5ar1-A (RA) or antithrombin to the VEGF-A treated WT segments markedly attenuated the sprouting (1.07 mm²) versus 0.26 and 0.28 mm² (P<0.05) (FIG. 42D). The results of multiple repeated assays are summarized in FIG. 42E. The inclusion of vorapaxar with VEGF-A had a similar blunting effect (FIG. 42F). Consistent with these results, aortic segments from C5ar1^{e/e} mice, double PAR3^{e/e}PAR4^{e/e} mice, and single PAR1^{e/e} mice (FIG. 42F) showed some sprouting in response to VEGF-A, but less than that of VEGF-A-treated WT segments. These ex vivo data are consistent with the confocal analyses in FIG. 36E of in vivo neo-angiogenesis in implanted Matrigel plugs showing FII, FV, FVII, and FX colocalizing in CD31 [symbol] cells. They are consistent with the multiple in vitro studies linking joint PAR1/4 and C5ar1 signaling with VEGF-A mitotic function detailed above. The data further argue that both PAR and C5ar1 signaling jointly regulate EC growth physiologically. A diagram depicting the linkage of endogenously produced thrombin and C5a with VEGF-A mitotic function in proliferating versus contact-inhibited ECs is shown in FIG. 42G. A second diagram depicting the linkage of combinatorial Gai-Gaq signaling by the interactive PAR4-C5ar1 functional complex with joint PKC activation/protein kinase A inactivation and PI-3Kγ/PI-3Ka signaling associated with VEGFR2 activation is shown in FIG. 42H.

Thrombin functions to repair injured blood vessels by generating fibrin and activating platelets that together seal separated vascular segments. In conjunction with this, it triggers EC proliferation that enables vascular reconstitution. The current view is that thrombin is solely generated in the intravascular compartment from prothrombinase components that are produced by the liver. Although a previous study found that thrombin can cleave C5a from complement component C5, the observation was connected with heightened plasma thrombin in disease. The studies herein provide the insight that this process operates endogenously in ECs to produce C5a and plays a requisite role in VEGF-A function.

The data show that in proliferating ECs: i) both coagulation factors, specifically FX, FV, FVII, and FII (pro-thrombin), which give rise to thrombin, and complement components, specifically C5 that gives rise to C5a, are jointly synthesized by ECs; and ii) thrombin (i.e., endogenously produced by ECs) jointly cleaves C5a from EC synthesized C5 and unmasks the tethered ligands of EC expressed PAR1 and/or PAR4, that together induce combinatorial C5ar1 and PAR4 signaling. They show that:

iii) the PAR4 and C5ar1 GPCRs are directly or indirectly complexed with each other in ECs and that iv) cooperative C5ar1 and PAR signaling in ECs is integrally involved in VEGF-A mitotic function. The experiments additionally provide evidence that: v) the associated PAR4-C5ar1 complex promotes combinatorial Gai and Gaq signaling that induces PI-3KeAKTemTOR and STAT3 activation as well as PLC activation. Finally, they provide evidence that:

vi) C5ar1, PAR4, and PAR1 signaling all induce PI-3Kγ activation in short-term cultures, whereas C5ar1 signaling additionally induces PI-3Ka activation. All, however, promote PI-3Ka activation in long-term cultures. The proximity of C5ar1 and PAR4, in principle, could promote efficient induction of joint C5ar1 and PAR4 signaling by EC produced thrombin, dual processes needed for VEGF-A mitotic function. Taken together, the findings support a requisite role of endogenous coagulation factor synthesis in EC growth. Mechanistically, they document an interconnection of the EC synthesized coagulation factors with EC synthesized complement via EC produced thrombin and show that thrombin-induced signaling through associated C5ar1 and PAR4 plays an integral role in VEGF-A mitotic function.

As emphasized in results, it is important to highlight the difference between the studies investigating proliferating ECs herein and those by many others examining homeostatic ECs. Most in vitro studies of ECs have used HUVEC grown to confluence, such that the ECs are contact inhibited. That system is intended to model conditions that pertain in intact blood vessels. Conditions that model the reconstitution of severed vessels were achieved by titrating cell numbers such that the cultures always remained nonconfluent in the presence of added VEGF-A, helping to study effects on EC proliferation in the absence of effects of contact inhibition. The comparisons of growing versus confluent HUVEC showed that although C5ar1 and PAR signaling are robust in proliferating ECs, both are repressed in confluent ECs, raising the possibility that down-regulation of this joint signaling participates in the processes that halt cell growth in contact inhibition.

Although the synthesis/presentation of tissue factor by ECs has been documented in many past studies, its function uniformly has been connected solely with intravascular coagulation. A contemporaneous study detected FVII, FIX, and FX as well as FII in confluent cultures of HUVECs and other ECs subjected to the serum-free conditions and noted that EC-produced FX could be activated without adding plasma factors. The findings in that system likewise were interpreted solely in the context of coagulation. Other prior studies have reported EC production of factor B and factor H and coagulation factors FX and FVII, and similarly interpreted the findings in the context of intravascular complement activation and coagulation. The findings herein differ from these past findings in that they document the endogenous production by ECs of the full array of prothrombinase components, leading to the intracellular production of thrombin in ECs. This process is mechanistically inter-connected with VEGF-A mitotic function rather than with coagulation.

The finding herein that thrombin is produced intracellularly is supported by the intracellular fluorescence-activated cell sorting analyses of thrombin protein, and the confocal localization of FII in the bodies of ECs growing in vivo in Matrigel. In addition, it is supported by the ability of VEGF-A stimulated (primary) HAECs to support autocrine PAR1 and PAR4 signaling in serum-free cultures, the abrogation of EC growth in serum-starved cultures by PAR inhibitors, and the findings that ECs produce all prothrombinase components that generate thrombin. Disruption of EC growth by siRNA knockdowns of PAR1 and PAR4 in serum-free media provided direct confirmation of the involvement of this signaling in EC growth. Our findings that the locally produced thrombin triggers joint C5ar1 and PAR signaling and that this cooperative signaling is needed for VEGF-A function thus tie EC produced coagulation proteins to an intracellular signaling system that plays an important role in EC growth. Although this process is distinct from coagulation per se, EC proliferation is an essential process in vascular repair.

BRET, co-immunoprecipitation analyses, and confocal microscopy all indicated that PAR4 and C5ar1 are intimately associated with each other (within 10 nm). The BRET studies did not detect proximity of PAR1 with C5ar1. However, the current findings that PAR1 antagonism (vorapaxar) inhibited EC proliferation, and that PAR1 agonists (TFLLRN or TP508) augmented EC proliferation, all implicated PAR1 signaling in VEGF-A function. The findings that PAR1 knockdown abolished VEGF-A induction of EC growth in serum-free media and that PAR1 genetic deficiency blunted sprouting further support this. Although the data for TP508 are controversial because a previous study indicated that TP508 activates ECs via binding to avb3 integrin, the combined data herein implicate TP508 in impacting PAR1 signaling. One possibility is that PAR1, which has high affinity for thrombin, is close to the PAR4-C5ar1 complex, allowing the tethered ligand of PAR1 to deliver thrombin to PAR4, similar to the reported cooperativity of murine PAR3 delivering thrombin to PAR4. Although possible, it is unlikely that the tethered ligand of PAR1 activates PAR4 because the sequences are different. A second possibility is that PAR1 and PAR4-C5ar1 signaling converge downstream. A precedent exists for the cooperativity of PAR1 signaling with PAR4 signaling in several systems. Because of differences in the extracellular regions of PAR1 and PAR4 and the higher affinity of PAR1 for thrombin, the tethered ligand of PAR4 is much less efficiently cleaved by thrombin. However, cooperation of PAR1 and PAR4 improves thrombin activation of PAR4. Functionally important, thrombin characteristically elicits a short burst of PAR1 signaling that is followed by a more prolonged expanse of PAR4 signaling.

The finding that C5ar1 and PAR4 are associated with each other could account for past reports that C5ar1 GPCR signaling, although usually being Gi coupled (pertussis toxin sensitive) and adenylyl cyclase repressive, can also activate PLC. Some studies have attributed the PLC activation to the by subunit of the C5ar1 G protein, whereas other studies have attributed it to alternative Gaq coupling. In the same vein, some studies have reported that PAR4, although generally being Gaq coupled, sometimes can be Gai coupled. Studies in platelets have found that PAR4 can couple the purinergic receptor P2Y12 that conveys Gai signaling. The current findings that C5ar1 is associated with PAR4 in ECs and that C5ar1 antagonism blocks thrombin suppression of protein kinase A, activation of AKT, and VEGFR2 signaling support the concept that in ECs the C5ar1-PAR4 complex could account for Gaq coupling of C5ar1 and Gai coupling of PAR4. More studies will be needed to definitively characterize the interactions in ECs versus platelets of PAR4 with C5ar1 or with P2Y12.

Whether EC thrombin generation and intravascular thrombin generation are entirely separate processes or whether they function together remains to be determined. A corollary of this distinction is whether EC produced thrombin contributes to intravascular coagulation and intravascularly produced thrombin contributes to VEGF-A induction of EC growth. Our prior studies of immune cell activation using bone marrow chimeras consisting of wild-type bone marrow in complement-deficient recipients and vice versa showed that immune cell produced and (liver-produced) systemic complement function independently of each other in separate compartments. These results underline the functional importance of distinguishing between autocrine and paracrine receptor tyrosine kinase, PAR, and C5ar1 signaling loops operating to promote EC growth. The current data will provide a framework for more definitively unraveling the respective involvement of systemic versus EC produced thrombin in EC growth.

In conclusion, the data herein shed light on several past observations. First and foremost, endothelium has the means to synthesize endogenous complement and coagulation proteins that function to promote cell growth and angiogenesis. Recognizing this has important consequences is essential because blockade of both thrombin and C5ar1 is needed to reduce inflammation and brain edema following intracerebral hemorrhage. Also, the PAR and C5ar1 receptor pathways are both involved in the mobilization of hematopoietic stem/progenitor cells, the inflammatory response that follows traumatic lung injury, and the expression of P-selectin by ECs.

### EXAMPLE 4

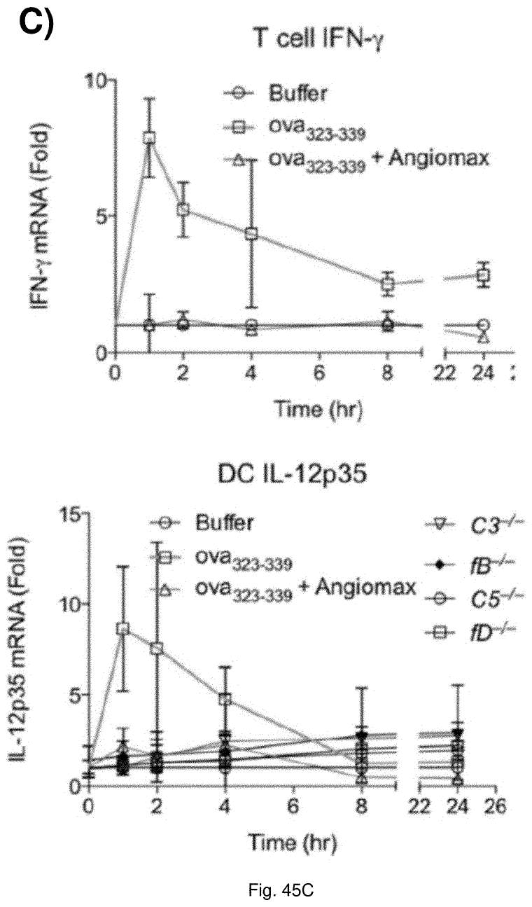

Relevant to our linkage that endogenously produced C3a and C5a and consequent C3ar1/C5ar1 signaling drives both B cell and T cell immunity, the finding that EC production of C5a depends on thrombin prompted the intriguing idea that the same process might operate in immune cells to promote T cell activation and Teff commitment. To test this idea, we first incubated purified CD4⁺ T cells with anti-CD3/28-Dynabeads in the presence of the thrombin specific inhibitor Angiomax (5-25 ug/ml) using C3ar1-A/C5ar1-A and media alone as +/−controls and after 48 h we measured T cell proliferation (FIG. 45A left, center, right). Strikingly, the thrombin blockade abolished T cell proliferation as profoundly as the C3ar1/C5ar1 blockade. The same results were observed at 72 h for T cell C5a production and IFN-γ production, indicative of thrombin blocking the Teff Th1 response. To test this effect on an authentic T cell response, we examined the integrated response of ova specific OT-II T cells to ova peptide and DCs. A comparable abrogating effect was observed in both the OT II cells on IFN-γ production and in the DC on IL-12/p35 production (FIGS. 45B, C). A corresponding effect was observed with B cell activation.

From the above description of the invention, those skilled in the art will perceive improvements, changes and modifications. Such improvements, changes and modifications are within the skill of the art and are intended to be covered by the appended claims. All publications, patents, and patent applications cited in the present application are herein incorporated by reference in their entirety.

Having described the invention, we claim:

1. A method of modulating T cell activation and/proliferation, the method comprising:
administering to the cells an amount of a thrombin inhibitor effective to modulate C3aR and/or C5aR signaling of the cells.

2. The method of claim 1, wherein the cells are in vasculature of the subject, proximate or about the periphery of a vascular injury, and the thrombin inhibitor is administered to the subject to inhibit at least one of T cell activation and/proliferation following stimulation of the T cells.

3. A method of inhibiting neointimal formation in the vasculature of a subject as a result of a vascular injury, the method comprising:
administering a thrombin inhibitor to at least one of endothelial cells or smooth muscle cells at the site of or proximate the site of the injured vasculature.

4. The method of claim 3, the agent being administered at an amount effect to inhibit at least one of PDGF production by the cells, cell growth, cell viability, or cell mitosis.

5. The method of claim 3, wherein the agent is administered at an amount effective to inhibit at least one of atherogenesis, thrombosis, or restinosis of the subject.

6. A method of inhibiting aberrant angiogenesis and/or vasculogenesis in tissue of a subject, the method comprising:
administering to the tissue of the subject a thrombin inhibitor.

7. The method of claim 6, the tissue comprising tumor tissue and the thrombin inhibitor being administered at an amount effect to inhibit aberrant tumor growth.

8. The method of claim 6, the tissue comprising choroid tissue and the thrombin inhibitor being administered at an amount effective to treat age-related macular degeneration.
